SEQUENCE LISTING (136 sequences)

SEQ ID NO: 1 - DNA, 20 bp, synthetic construct
  source 1..20: mol_type = other DNA
gttcgtttagagaacagatc

SEQ ID NO: 2 - DNA, 21 bp, synthetic construct
  source 1..21: mol_type = other DNA
taaagttcgtttagagaacag

SEQ ID NO: 3 - DNA, 15 bp, synthetic construct
  source 1..15: mol_type = other DNA
agccctgtatacgac

SEQ ID NO: 4 - DNA, 19 bp, synthetic construct
  source 1..19: mol_type = other DNA
gtaggtaaaaacctaatat

SEQ ID NO: 5 - DNA, 20 bp, synthetic construct
  source 1..20: mol_type = other DNA
cgtttagagaacagatctac

SEQ ID NO: 6 - DNA, 20 bp, synthetic construct
  source 1..20: mol_type = other DNA
cattgtagatgtcaaaagcc

SEQ ID NO: 7 - DNA, 21 bp, synthetic construct
  source 1..21: mol_type = other DNA
ctccctcatggtggcagttga

SEQ ID NO: 8 - DNA, 18 bp, synthetic construct
  source 1..18: mol_type = other DNA
cggcatgtctattttgta

SEQ ID NO: 9 - DNA, 18 bp, synthetic construct
  source 1..18: mol_type = other DNA
tttgttcctggatgactc

SEQ ID NO: 10 - DNA, 18 bp, synthetic construct
  source 1..18: mol_type = other DNA
gtttgttcctggatgact

SEQ ID NO: 11 - DNA, 18 bp, synthetic construct
  source 1..18: mol_type = other DNA
agtttgttcctggatgac

SEQ ID NO: 12 - DNA, 18 bp, synthetic construct
  source 1..18: mol_type = other DNA
cagtttgttcctggatga

SEQ ID NO: 13 - DNA, 18 bp, synthetic construct
  source 1..18: mol_type = other DNA
tcagtttgttcctggatg

SEQ ID NO: 14 - DNA, 18 bp, synthetic construct
  source 1..18: mol_type = other DNA
ctcagtttgttcctggat

SEQ ID NO: 15 - DNA, 18 bp, synthetic construct
  source 1..18: mol_type = other DNA
tgtgtgtgtgtgcgtgtg

SEQ ID NO: 16 - DNA, 18 bp, synthetic construct
  source 1..18: mol_type = other DNA
gtgtgtgtgtgtgcgtgt

SEQ ID NO: 17 - DNA, 18 bp, synthetic construct
  source 1..18: mol_type = other DNA
tgtgtgtgtgtgtgcgtg

SEQ ID NO: 18 - DNA, 18 bp, synthetic construct
  source 1..18: mol_type = other DNA
gtgtgtgtgtgtgtgcgt

SEQ ID NO: 19 - DNA, 18 bp, synthetic construct
  source 1..18: mol_type = other DNA
tgtgtgtgtgtgtgtgcg

SEQ ID NO: 20 - DNA, 18 bp, synthetic construct
  source 1..18: mol_type = other DNA
gtgtgtgtgtgtgtgtgc

SEQ ID NO: 21 - DNA, 18 bp, synthetic construct
  source 1..18: mol_type = other DNA
tgtgtgtgtgtgtgtgtg

SEQ ID NO: 22 - DNA, 18 bp, synthetic construct
  source 1..18: mol_type = other DNA
gtgtgtgtgtgtgtgtgt

SEQ ID NO: 23 - DNA, 18 bp, synthetic construct
  source 1..18: mol_type = other DNA
tgtgtgtgtgtgtgtgtg

SEQ ID NO: 24 - DNA, 18 bp, synthetic construct
  source 1..18: mol_type = other DNA
gtgtgtgtgtgtgtgtgt

SEQ ID NO: 25 - DNA, 18 bp, synthetic construct
  source 1..18: mol_type = other DNA
tgtgtgtgtgtgtgtgtg

SEQ ID NO: 26 - DNA, 18 bp, synthetic construct
  source 1..18: mol_type = other DNA
gtgtgtgtgtgtgtgtgt

SEQ ID NO: 27 - DNA, 18 bp, synthetic construct
  source 1..18: mol_type = other DNA
tgtgtgtgtgtgtgtgtg

SEQ ID NO: 28 - DNA, 18 bp, synthetic construct
  source 1..18: mol_type = other DNA
gtgtgtgtgtgtgtgtgt

SEQ ID NO: 29 - DNA, 18 bp, synthetic construct
  source 1..18: mol_type = other DNA
cgtgtgtgtgtgtgtgtg

SEQ ID NO: 30 - DNA, 18 bp, synthetic construct
  source 1..18: mol_type = other DNA
gcgtgtgtgtgtgtgtgt

SEQ ID NO: 31 - DNA, 18 bp, synthetic construct
  source 1..18: mol_type = other DNA
tgcgtgtgtgtgtgtgtg

SEQ ID NO: 32 - DNA, 18 bp, synthetic construct
  source 1..18: mol_type = other DNA
gtgcgtgtgtgtgtgtgt

SEQ ID NO: 33 - DNA, 18 bp, synthetic construct
  source 1..18: mol_type = other DNA
agtggcggatctgaactc

SEQ ID NO: 34 - DNA, 18 bp, synthetic construct
  source 1..18: mol_type = other DNA
gagtggcggatctgaact

SEQ ID NO: 35 - DNA, 18 bp, synthetic construct
  source 1..18: mol_type = other DNA
gagtgtgctgcaggtaga

SEQ ID NO: 36 - DNA, 18 bp, synthetic construct
  source 1..18: mol_type = other DNA
tcgagtgtgctgcaggta

SEQ ID NO: 37 - DNA, 18 bp, synthetic construct
  source 1..18: mol_type = other DNA
atcgagtgtgctgcaggt

SEQ ID NO: 38 - DNA, 18 bp, synthetic construct
  source 1..18: mol_type = other DNA
gtgctgttgtagatggaa

SEQ ID NO: 39 - DNA, 18 bp, synthetic construct
  source 1..18: mol_type = other DNA
ggtgctgttgtagatgga

SEQ ID NO: 40 - DNA, 18 bp, synthetic construct
  source 1..18: mol_type = other DNA
tggtgctgttgtagatgg

SEQ ID NO: 41 - DNA, 18 bp, synthetic construct
  source 1..18: mol_type = other DNA
ctggtgctgttgtagatg

SEQ ID NO: 42 - DNA, 18 bp, synthetic construct
  source 1..18: mol_type = other DNA
cctggtgctgttgtagat

SEQ ID NO: 43 - DNA, 18 bp, synthetic construct
  source 1..18: mol_type = other DNA
ccctggtgctgttgtaga

SEQ ID NO: 44 - DNA, 18 bp, synthetic construct
  source 1..18: mol_type = other DNA
tccctggtgctgttgtag

SEQ ID NO: 45 - DNA, 18 bp, synthetic construct
  source 1..18: mol_type = other DNA
gtccctggtgctgttgta

SEQ ID NO: 46 - DNA, 18 bp, synthetic construct
  source 1..18: mol_type = other DNA
agtccctggtgctgttgt

SEQ ID NO: 47 - DNA, 18 bp, synthetic construct
  source 1..18: mol_type = other DNA
ctgggttggagatgttaa

SEQ ID NO: 48 - DNA, 18 bp, synthetic construct
  source 1..18: mol_type = other DNA
gctgggttggagatgtta

SEQ ID NO: 49 - DNA, 18 bp, synthetic construct
  source 1..18: mol_type = other DNA
cgctgggttggagatgtt

SEQ ID NO: 50 - DNA, 18 bp, synthetic construct
  source 1..18: mol_type = other DNA
tagcgctgggttggagat

SEQ ID NO: 51 - DNA, 18 bp, synthetic construct
  source 1..18: mol_type = other DNA
atgtagcgctgggttgga

SEQ ID NO: 52 - DNA, 18 bp, synthetic construct
  source 1..18: mol_type = other DNA
agccattcgccttctgct

SEQ ID NO: 53 - DNA, 18 bp, synthetic construct
  source 1..18: mol_type = other DNA
gtctttatggtgaagcca

SEQ ID NO: 54 - DNA, 18 bp, synthetic construct
  source 1..18: mol_type = other DNA
tgtctttatggtgaagcc

SEQ ID NO: 55 - DNA, 18 bp, synthetic construct
  source 1..18: mol_type = other DNA
ctgtctttatggtgaagc

SEQ ID NO: 56 - DNA, 18 bp, synthetic construct
  source 1..18: mol_type = other DNA
cctgtctttatggtgaag

SEQ ID NO: 57 - DNA, 18 bp, synthetic construct
  source 1..18: mol_type = other DNA
gttcctgtctttatggtg

SEQ ID NO: 58 - DNA, 18 bp, synthetic construct
  source 1..18: mol_type = other DNA
ggttcctgtctttatggt

SEQ ID NO: 59 - DNA, 18 bp, synthetic construct
  source 1..18: mol_type = other DNA
aggttcctgtctttatgg

SEQ ID NO: 60 - DNA, 18 bp, synthetic construct
  source 1..18: mol_type = other DNA
caggttcctgtctttatg

SEQ ID NO: 61 - DNA, 18 bp, synthetic construct
  source 1..18: mol_type = other DNA
ccaggttcctgtctttat

SEQ ID NO: 62 - DNA, 18 bp, synthetic construct
  source 1..18: mol_type = other DNA
ggtcttcccactgttttt

SEQ ID NO: 63 - DNA, 18 bp, synthetic construct
  source 1..18: mol_type = other DNA
aggagatgtggggtcttc

SEQ ID NO: 64 - DNA, 18 bp, synthetic construct
  source 1..18: mol_type = other DNA
caggagatgtggggtctt

SEQ ID NO: 65 - DNA, 18 bp, synthetic construct
  source 1..18: mol_type = other DNA
ggttggtctgttgtgact

SEQ ID NO: 66 - DNA, 18 bp, synthetic construct
  source 1..18: mol_type = other DNA
cggttggtctgttgtgac

SEQ ID NO: 67 - DNA, 18 bp, synthetic construct
  source 1..18: mol_type = other DNA
ccggttggtctgttgtga

SEQ ID NO: 68 - DNA, 18 bp, synthetic construct
  source 1..18: mol_type = other DNA
gagaatggttagaggttc

SEQ ID NO: 69 - DNA, 18 bp, synthetic construct
  source 1..18: mol_type = other DNA
gtagagaatggttagagg

SEQ ID NO: 70 - DNA, 18 bp, synthetic construct
  source 1..18: mol_type = other DNA
ggtgttttgccaatgtag

SEQ ID NO: 71 - DNA, 18 bp, synthetic construct
  source 1..18: mol_type = other DNA
gggtgttttgccaatgta

SEQ ID NO: 72 - DNA, 18 bp, synthetic construct
  source 1..18: mol_type = other DNA
tgggtgttttgccaatgt

SEQ ID NO: 73 - DNA, 18 bp, synthetic construct
  source 1..18: mol_type = other DNA
catcatcgttgtcgtcgt

SEQ ID NO: 74 - DNA, 18 bp, synthetic construct
  source 1..18: mol_type = other DNA
gaacggtacgtacagcaa

SEQ ID NO: 75 - DNA, 18 bp, synthetic construct
  source 1..18: mol_type = other DNA
ggaacggtacgtacagca

SEQ ID NO: 76 - DNA, 18 bp, synthetic construct
  source 1..18: mol_type = other DNA
aggaacggtacgtacagc

SEQ ID NO: 77 - DNA, 18 bp, synthetic construct
  source 1..18: mol_type = other DNA
taggaacggtacgtacag

SEQ ID NO: 78 - DNA, 18 bp, synthetic construct
  source 1..18: mol_type = other DNA
ataggaacggtacgtaca

SEQ ID NO: 79 - DNA, 18 bp, synthetic construct
  source 1..18: mol_type = other DNA
gataggaacggtacgtac

SEQ ID NO: 80 - DNA, 18 bp, synthetic construct
  source 1..18: mol_type = other DNA
ggataggaacggtacgta

SEQ ID NO: 81 - DNA, 18 bp, synthetic construct
  source 1..18: mol_type = other DNA
gggataggaacggtacgt

SEQ ID NO: 82 - DNA, 18 bp, synthetic construct
  source 1..18: mol_type = other DNA
gggtgcctattgcatagc

SEQ ID NO: 83 - DNA, 18 bp, synthetic construct
  source 1..18: mol_type = other DNA
agggtgcctattgcatag

SEQ ID NO: 84 - DNA, 18 bp, synthetic construct
  source 1..18: mol_type = other DNA
aagggtgcctattgcata

SEQ ID NO: 85 - DNA, 18 bp, synthetic construct
  source 1..18: mol_type = other DNA
gaagggtgcctattgcat

SEQ ID NO: 86 - DNA, 18 bp, synthetic construct
  source 1..18: mol_type = other DNA
ggaagggtgcctattgca

SEQ ID NO: 87 - DNA, 18 bp, synthetic construct
  source 1..18: mol_type = other DNA
tgggaagggtgcctattg

SEQ ID NO: 88 - DNA, 18 bp, synthetic construct
  source 1..18: mol_type = other DNA
atgggaagggtgcctatt

SEQ ID NO: 89 - DNA, 18 bp, synthetic construct
  source 1..18: mol_type = other DNA
aatgggaagggtgcctat

SEQ ID NO: 90 - DNA, 18 bp, synthetic construct
  source 1..18: mol_type = other DNA
gaatgggaagggtgccta

SEQ ID NO: 91 - DNA, 18 bp, synthetic construct
  source 1..18: mol_type = other DNA
agaatgggaagggtgcct

SEQ ID NO: 92 - DNA, 18 bp, synthetic construct
  source 1..18: mol_type = other DNA
aagaatgggaagggtgcc

SEQ ID NO: 93 - DNA, 18 bp, synthetic construct
  source 1..18: mol_type = other DNA
taagaatgggaagggtgc

SEQ ID NO: 94 - DNA, 18 bp, synthetic construct
  source 1..18: mol_type = other DNA
gtaagaatgggaagggtg

SEQ ID NO: 95 - DNA, 18 bp, synthetic construct
  source 1..18: mol_type = other DNA
agtaagaatgggaagggt

SEQ ID NO: 96 - DNA, 18 bp, synthetic construct
  source 1..18: mol_type = other DNA
gagtaagaatgggaaggg

SEQ ID NO: 97 - DNA, 18 bp, synthetic construct
  source 1..18: mol_type = other DNA
cagactttctcggtcata

SEQ ID NO: 98 - DNA, 18 bp, synthetic construct
  source 1..18: mol_type = other DNA
gcagactttctcggtcat

SEQ ID NO: 99 - DNA, 18 bp, synthetic construct
  source 1..18: mol_type = other DNA
tgcagactttctcggtca

SEQ ID NO: 100 - DNA, 18 bp, synthetic construct
  source 1..18: mol_type = other DNA
atgcagactttctcggtc

SEQ ID NO: 101 - DNA, 18 bp, synthetic construct
  source 1..18: mol_type = other DNA
aatgcagactttctcggt

SEQ ID NO: 102 - DNA, 18 bp, synthetic construct
  source 1..18: mol_type = other DNA
taatgcagactttctcgg

SEQ ID NO: 103 - DNA, 18 bp, synthetic construct
  source 1..18: mol_type = other DNA
gacctggacttttttccc

SEQ ID NO: 104 - DNA, 18 bp, synthetic construct
  source 1..18: mol_type = other DNA
tgacctggacttttttcc

SEQ ID NO: 105 - DNA, 18 bp, synthetic construct
  source 1..18: mol_type = other DNA
ctgacctggacttttttc

SEQ ID NO: 106 - DNA, 18 bp, synthetic construct
  source 1..18: mol_type = other DNA
gctgacctggactttttt

SEQ ID NO: 107 - DNA, 18 bp, synthetic construct
  source 1..18: mol_type = other DNA
ctgcaatgatgtggcaaa

SEQ ID NO: 108 - DNA, 18 bp, synthetic construct
  source 1..18: mol_type = other DNA
tctgcaatgatgtggcaa

SEQ ID NO: 109 - DNA, 18 bp, synthetic construct
  source 1..18: mol_type = other DNA
ttctgcaatgatgtggca

SEQ ID NO: 110 - DNA, 18 bp, synthetic construct
  source 1..18: mol_type = other DNA
cttctgcaatgatgtggc

SEQ ID NO: 111 - DNA, 18 bp, synthetic construct
  source 1..18: mol_type = other DNA
gctgcccacttgcatact

SEQ ID NO: 112 - DNA, 18 bp, synthetic construct
  source 1..18: mol_type = other DNA
gttggcagaacatagaac

SEQ ID NO: 113 - DNA, 18 bp, synthetic construct
  source 1..18: mol_type = other DNA
cgttggcagaacatagaa

SEQ ID NO: 114 - DNA, 18 bp, synthetic construct
  source 1..18: mol_type = other DNA
gcgttggcagaacataga

SEQ ID NO: 115 - DNA, 18 bp, synthetic construct
  source 1..18: mol_type = other DNA
atggggctacaggggata

SEQ ID NO: 116 - DNA, 18 bp, synthetic construct
  source 1..18: mol_type = other DNA
tatggggctacaggggat

SEQ ID NO: 117 - DNA, 18 bp, synthetic construct
  source 1..18: mol_type = other DNA
ttatggggctacagggga

SEQ ID NO: 118 - DNA, 18 bp, synthetic construct
  source 1..18: mol_type = other DNA
agttatggggctacaggg

SEQ ID NO: 119 - DNA, 18 bp, synthetic construct
  source 1..18: mol_type = other DNA
aagttatggggctacagg

SEQ ID NO: 120 - DNA, 18 bp, synthetic construct
  source 1..18: mol_type = other DNA
caagttatggggctacag

SEQ ID NO: 121 - DNA, 18 bp, synthetic construct
  source 1..18: mol_type = other DNA
ccaagttatggggctaca

SEQ ID NO: 122 - DNA, 18 bp, synthetic construct
  source 1..18: mol_type = other DNA
tccaagttatggggctac

SEQ ID NO: 123 - DNA, 18 bp, synthetic construct
  source 1..18: mol_type = other DNA
atccaagttatggggcta

SEQ ID NO: 124 - DNA, 18 bp, synthetic construct
  source 1..18: mol_type = other DNA
tatccaagttatggggct

SEQ ID NO: 125 - DNA, 18 bp, synthetic construct
  source 1..18: mol_type = other DNA
ctatccaagttatggggc

SEQ ID NO: 126 - DNA, 18 bp, synthetic construct
  source 1..18: mol_type = other DNA
attggaggaaatagggtg

SEQ ID NO: 127 - DNA, 18 bp, synthetic construct
  source 1..18: mol_type = other DNA
gtcttgtaggtagcagcc

SEQ ID NO: 128 - DNA, 18 bp, synthetic construct
  source 1..18: mol_type = other DNA
ggtcttgtaggtagcagc

SEQ ID NO: 129 - DNA, 18 bp, synthetic construct
  source 1..18: mol_type = other DNA
tggtcttgtaggtagcag

SEQ ID NO: 130 - DNA, 18 bp, synthetic construct
  source 1..18: mol_type = other DNA
ctggtcttgtaggtagca

SEQ ID NO: 131 - DNA, 18 bp, synthetic construct
  source 1..18: mol_type = other DNA
tctggtcttgtaggtagc

SEQ ID NO: 132 - DNA, 18 bp, synthetic construct
  source 1..18: mol_type = other DNA
gtctggtcttgtaggtag

SEQ ID NO: 133 - DNA, 18 bp, synthetic construct
  source 1..18: mol_type = other DNA
agtctggtcttgtaggta

SEQ ID NO: 134 - DNA, 18 bp, synthetic construct
  source 1..18: mol_type = other DNA
gagtctggtcttgtaggt

SEQ ID NO: 135 - DNA, 18 bp, synthetic construct
  source 1..18: mol_type = other DNA
ggagtctggtcttgtagg

SEQ ID NO: 136 - DNA, 18 bp, synthetic construct
  source 1..18: mol_type = other DNA
aggagtctggtcttgtag

(19) United States
(12) Patent Application Publication
(10) Pub. No.: US 2025/0332340 A1
(43) Pub. Date: Oct. 30, 2025

(54) COMPOSITIONS AND METHODS FOR CNS DISEASES

(71) Applicant: GMP BIOTECHNOLOGY LIMITED, Hong Kong (CN)

(72) Inventor: Vuong TRIEU, Agoura Hills, CA (US)

(21) Appl. No.: 19/201,668

(22) Filed: May 7, 2025

Related U.S. Application Data
(63) Continuation of application No. PCT/US2023/079070, filed on Nov. 8, 2023.
(60) Provisional application No. 63/383,032, filed on Nov. 9, 2022.
(60) Provisional application No. 63/383,453, filed on Nov. 11, 2022.
(60) Provisional application No. 63/384,089, filed on Nov. 16, 2022.
(60) Provisional application No. 63/384,088, filed on Nov. 16, 2022.
(60) Provisional application No. 63/449,286, filed on Mar. 1, 2023.
(60) Provisional application No. 63/451,220, filed on Mar. 9, 2023.

(51) Int. Cl. A61M 5/142; A61K 9/00; A61K 38/00; A61M 5/165; A61M 5/168; C07K 7/08

(52) U.S. Cl. CPC A61M 5/142; A61K 9/0019; A61M 5/165; A61M 5/16831; C07K 7/08; A61K 38/00; A61M 2005/1652; A61M 2205/05; A61M 2205/3334

(57) ABSTRACT

This invention describes agents, methods, compositions, kits and uses of medicaments for inhibiting or suppressing expression of TGF-β2 for treating or ameliorating the symptoms of a CNS disease in a human subject or animal, including diffuse midline glioma (DMG) and K27M GBM. The agents may be used in combination with cancer drugs. These purposes can be achieved with formulations of agents for inhibiting or suppressing expression of TGF-β2. More particularly, this invention discloses compositions, methods and uses for antisense oligonucleotides against TGF-β2, in a regimen for CNS disease. This invention further describes novel devices and methods for delivering a pharmaceutical composition by intracranial infusion.

### SEQUENCE LISTING

This application includes a sequence listing submitted electronically as an ST.26 file created on May 6, 2025, named 018988-006US1_SL.xml, which is 120,186 bytes in size.

### TECHNICAL FIELD

This invention relates to methods, agents and uses for treating central nervous system (CNS) cancers by continuous intracranial infusion. More particularly, this invention discloses methods, agents and uses for inhibiting or suppressing TGF-β2, which provide improved clinical outcomes for such diseases. This invention provides stable formulations of anti-TGF-β2 agents including antisense oligonucleotide compositions and other agents and methods of use for treating CNS cancers.

This invention relates to devices and methods for delivering a pharmaceutical composition by intracranial infusion. More particularly, a device of this invention may include an ommaya-like reservoir into which is pumped a pharmaceutical composition. The ommaya-like reservoir can provide a range of intracranial infusion, from bolus to continuous, via an entry catheter, which enters intraventricularly into a target region of the brain.

### BACKGROUND

CNS diseases have been treated with various agents through intracranial routes. For CNS tumors, a convection enhanced delivery (CED) device can be used for distribution of drug directly to a tumor in the brain using several catheters positioned close to the tumor.

However, drawbacks and complications of these infusion methods are that (a) placement of the catheter and its tip into the brain near the tumor is complex and typically requires concurrent imaging such as x-ray based imaging to be achieved, (b) the methods require multiple catheters to be inserted for tumors which is difficult or impossible to achieve and may cause tissue disruption, (c) the catheters cannot be implanted permanently because the brain will enlarge, especially for pediatric patients, the tumor will shift, and the tumor will become metastasized, and (d) a majority of tumors would already have metastasized within the CNS or outside the CNS making localized treatment moot.

Further, drawbacks and complications of such methods are that infusion and flow of the drug throughout the CNS compartments is limited.

Additional drawbacks are that an intratumorally-implanted catheter tip may not provide sufficient infusion and flow of drug throughout the CNS compartments.

What is needed is a device for intracranial systemic, bolus and/or continuous infusion of therapeutics for use in methods for treating central nervous system (CNS) diseases including cancer.

What is needed are methods and agents for use in inhibiting or suppressing factors in the unpredictable pathology of diseases of the central nervous system, in particular cancer. For example, there is an urgent need for methods and agents for use in inhibiting the activity of TGF-β and/or suppressing TGF-β related pathologies, which can improve the efficacy for treating central nervous system cancers.

### BRIEF SUMMARY

This invention provides therapies for treating or ameliorating symptoms of CNS disease such as CNS cancer.

In some embodiments, this invention includes agents and compositions for inhibiting or suppressing TGF-β2 to provide improved clinical outcomes for CNS disease.

In further embodiments, this invention provides stable formulations of anti-TGF-β2 agents for various therapies for CNS disease. Examples of anti-TGF-β2 agents include TGF-β2 inhibitors such as antisense oligonucleotides, pharmaceutically acceptable salts forms, esters, polymorphs or stereoisomers thereof, as well as combinations thereof.

In further aspects, this disclosure provides highly stable formulations of anti-TGF-β2 agents for therapies for CNS disease. The stable formulations of this invention provide surprisingly improved clinical results. Stable formulations of agents for suppressing TGF-β2 can be used to treat CNS disease, in particular CNS cancer.

This invention provides a device and system for intracranial, intrathecal or intraventricular continuous infusion of therapeutics for treating central nervous system (CNS) diseases. Continuous infusion to brain tumors with the system of this invention is far easier than for conventional CED systems and other similar or conventional methods.

The infusion system of this invention has a significant advantage because it delivers an effective amount or dose of a pharmaceutical composition continuously to a patient with much greater safety and ease of insertion throughout the CNS. For example, the infusion system of this invention will deliver by an intraventricular route a more effective amount of the drug throughout the CNS/brain compartments versus localized delivery by CED methods. This advantage can be significant especially for treatment of CNS cancers.

A device of this invention can overcome the drawbacks and complications of conventional methods. The infusion system of this invention has significant advantages because placement of the catheter and its tip into the brain is relatively easy and requires no imaging to be accomplished. Further, the infusion and flow of the drug to the brain takes advantage of fluids circulation in the brain to distribute the drug effectively over a larger area and to targeted tissues. Moreover, the system of this invention can be used in a novel mode of operation to provide continuous infusion to the targeted tissues.

This invention also relates to a device and system for intracranial, intrathecal or intraventricular continuous infusion of therapeutics for treating central nervous system (CNS) diseases of the brain and spine. The system of this invention will deliver compositions and agents for inhibiting or suppressing TGF-β2 to improve clinical outcomes for such diseases. This system can use stable formulations of anti-TGF-β2 agents including antisense oligonucleotide compositions and other agents for treating a CNS disease including CNS cancers.

This invention further relates to methods for treating for treating central nervous system (CNS) diseases of the brain and spine.

This invention further provides a device for intracranial, intrathecal or intraventricular continuous infusion of therapeutics for treating central nervous system (CNS) diseases. Continuous infusion to brain tumors with the device and device of this invention is far easier than for conventional CED methods and other similar or conventional methods.

The infusion device of this invention has a significant advantage because it delivers an effective amount or dose of a pharmaceutical composition continuously to a patient with much greater safety and ease of insertion throughout the CNS. For example, the infusion device and device of this invention will deliver by an intraventricular route a more effective amount of the drug throughout the CNS/brain compartments versus localized delivery by CED methods. This advantage can be significant especially for treatment of CNS cancers.

The device of this invention overcomes the drawbacks and complications of known methods. The infusion device of this invention has significant advantages because placement of the catheter and its tip into the brain is relatively easy and requires no imaging to be accomplished. Further, the infusion and flow of the drug to the brain takes advantage of fluids circulation in the brain to distribute the drug effectively over a larger area and to targeted tissues. Moreover, the device of this invention can be used in a novel mode of operation to provide continuous infusion to the targeted tissues.

This invention also relates to a device for intracranial, intrathecal or intraventricular continuous infusion of therapeutics for treating central nervous system (CNS) diseases of the brain and spine. The device of this invention will deliver compositions and agents for inhibiting or suppressing TGF-β2 to improve clinical outcomes for such diseases. This device uses stable formulations of anti-TGF-β2 agents including antisense oligonucleotide compositions and other agents for treating a CNS disease including CNS cancers.

This invention further relates to methods for treating for treating central nervous system (CNS) diseases of the brain and spine.

Embodiments of this invention include the following:

An agent for inhibiting or suppressing expression of TGF-β2 for treating or ameliorating the symptoms of a CNS disease in a human subject or animal.

Use of an agent for inhibiting or suppressing expression of TGF-β2 in the preparation of a medicament for treating or ameliorating the symptoms of a CNS disease in a human subject or animal.

A method for treating or ameliorating the symptoms of a CNS disease in a human subject or animal in need, the method comprising:
preparing a composition comprising an agent for inhibiting or suppressing expression of TGF-β2 in a carrier; and
administering a therapeutically effective amount of the composition to the subject.

The agent, use or method above, wherein the CNS disease is glioma, glioblastoma, diffuse intrinsic pontine glioma (DIPG), diffuse midline glioma (DMG), leptomeningeal or brain metastasis, brain or spinal cancer, or CNS tumors.

The agent, use or method above, in combination with a medicament comprising a targeted cancer drug, a cancer growth blocker, an EGFR inhibitor, or a combination thereof.

The agent, use or method above, in combination with a medicament selected from bevacizumab, everolimus, belzutifan, dabrafenib, trametinib, and combinations thereof.

The agent, use or method above, in combination with a medicament which is a cancer growth blocker selected from an angiogenesis inhibitor, a histone deacetylase inhibitor, a hedgehog blocker, an mTOR inhibitor, a p53 inhibitor, a PARP inhibitor, a proteasome inhibitor, a tyrosine kinase inhibitor, and combinations thereof.

The agent, use or method above, in combination with a medicament which is an EGFR inhibitor selected from erlotinib, gefitinib, afatinib, osimertinib, dacomitininb, and combinations thereof.

The agent, use or method above, in combination with temozolomide.

The agent, use or method above, in combination with treatment of the CNS disease by radiation therapy or electric field therapy.

The agent, use or method above, wherein the administration or use of the composition or agent is combined with a standard of care treatment for the CNS disease.

The agent, use or method above, wherein the agents, medicaments, therapies, treatments, and administrations are each administered concurrently, simultaneously, sequentially, or separately in time.

The agent, use or method above, wherein each agent and medicament is administered separately or in combination by infusion or injection.

The agent, use or method above, comprising administration or use by intracranial continuous infusion or bolus administration.

The agent, use or method above, wherein the intracranial continuous infusion comprises infusion with an ommaya-like reservoir having a partially-flexible top.

The agent, use or method above, wherein the intracranial continuous infusion comprises a single entry catheter placed into a target region of brain.

The agent, use or method above, wherein the subject upon the administration or use has an improved TGF-β2 signature.

The agent, use or method above, wherein the administration or use decreases mortality rate at month 6, 12, 18, 24, 30, or 36.

The agent, use or method above, wherein the administration or use increases survival rate at month 6, 12, 18, 24, 30, or 36.

The agent, use or method above, wherein the agent for inhibiting or suppressing expression of TGF-β2 is selected from TGF-β2-specific antisense oligonucleotides complementary to a TGF-β2 transcript as follows: Table 2 SEQ ID NOs:1-136 and chemically-modified variants thereof, LNA variants thereof, gapmer variants thereof, and any combination thereof.

The agent, use or method above, wherein the agent for inhibiting or suppressing expression of TGF-β2 is a TGF-β2-specific antisense oligonucleotide having no more than one or two mismatches as compared to a target human TGF-β2.

The agent, use or method above, wherein the agent for inhibiting or suppressing expression of TGF-β2 is a TGF-β2-specific antisense oligonucleotide which reduces a TGF-β2 transcript level by at least 60%, or at least 70%, or at least 80%, or at least 90%.

The agent, use or method above, wherein the agent for inhibiting or suppressing expression of TGF-β2 is a TGF-β2-specific antisense oligonucleotide which reduces any TGF-β1 transcript level and any TGF-β3 transcript level by less than 10%, or less than 5%, or less than 1%.

The agent, use or method above, comprising a TGF-β2-specific antisense oligonucleotide having one or more nucleotides chemically modified as a phosphorothioate internucleoside linkage, a methoxypropylphosphonate internucleoside linkage, an aminophosphoro linkage to a morpholino group, a 2′-OMe ribose group, a 2′-MOE methoxyethyl ribose group, a 2′-4′ constrained methoxyethyl bicyclic ribose group, a 2′-4′ constrained ethyl bicyclic ribose group, an LNA ribose group, a 2′-F ribose group, or a 5-methylcytodine base.

The agent, use or method above, wherein the agent is conjugated to a polyethylene glycol, a lipid, or a triantenarry N-acteyl-galactosamine.

The agent, use or method above, comprising a carrier of sterile water for injection, saline, isotonic saline, phosphate buffered saline, or a combination thereof.

The agent, use or method above, wherein the agent, medicament or administration is substantially free of excipients.

The agent, use or method above, wherein the agent, medicament or administration is stable for at least 14 days in carrier at 37° C. while being pumped in intracranial continuous infusion, or wherein there is less than 10% reduction in antisense active agent concentration after 90 days in-use.

The agent, use or method above, wherein the method comprises administering the composition by intracranial infusion at a rate of 2-8 μl/min and an agent concentration of 1-80 μM on Days 1 to 7, preferably for intracranial continuous infusion.

A kit comprising: an agent comprising a total of 250 mg of one or more TGF-β2-specific antisense oligonucleotides selected from Table 2 SEQ ID NOs:1-136 and chemically-modified variants thereof, LNA variants thereof, gapmer variants thereof, and any combination thereof; and
an ommaya-like reservoir having a partially-flexible top.

A device for delivering a fluid pharmaceutical composition by intracranial continuous infusion, the device comprising:
a reservoir [117] containing the pharmaceutical composition;
a pump [101] to force the pharmaceutical composition through an infusion tube
into an ommaya reservoir [111], the pump being in fluid communication with the ommaya reservoir, and wherein the pump is in fluid communication with the reservoir through a reservoir tube [115];
a filter [105] in line with the infusion tube; and
an entry catheter [113] in fluid communication with the ommaya reservoir, wherein the entry catheter is substantially linear and enters intraventricularly into a target region of the brain.

The device above, wherein the entry catheter [113] is non-linear and has one or more bends to enter intraventricularly into a target region of the brain.

A device for delivering a fluid pharmaceutical composition by intracranial continuous infusion, the device comprising:
a reservoir [117] containing the pharmaceutical composition;
a pump [101] to force the pharmaceutical composition through an infusion tube [103] into an access port [107], the pump being in fluid communication with an ommaya reservoir [111], wherein the reservoir is in fluid communication with the pump through a reservoir tube [115];
a filter [105] in line with the infusion tube;
an indwelling tube [109] in fluid communication with the access port and the ommaya reservoir [111]; and
an entry catheter [113] in fluid communication with the ommaya reservoir, wherein the entry catheter is substantially linear and enters intraventricularly into a target region of the brain.

The device above, wherein the entry catheter [113] is non-linear and has one or more bends to enter intraventricularly into a target region of the brain.

The device above, wherein the ommaya reservoir [111] comprises a partially-flexible top.

The device above, wherein the device provides continuous infusion of a therapeutically effective amount of the fluid pharmaceutical composition to the target region.

The device above, wherein the distal end of the entry catheter enters the target region of the brain.

The device above, wherein the ommaya reservoir [111] holds the pharmaceutical composition behind a membrane for a sustained period of time for sustained release of the pharmaceutical composition to the entry catheter.

The device above, wherein the distal end tip of the entry catheter that enters the brain is a step-down end, a recessed step end, a multi-port end, a microporous end, or a balloon tipped end.

The device above, wherein the pump [101] is a Pegasus Vario, a PEGA PCA, a CADD Solis VIP, a CADD-Legacy PLUS, a CADD-Legacy PCA, a CADD-Legacy 1, or other pump with similar specifications.

The device above, wherein the infusion rate of the fluid pharmaceutical composition is from 0.01 to 3000 ml/hr, or from 0.01 to 100 ml/hr, or from 0.01 to 2 ml/hr, or from 0.01 to 1 ml/hr, or from 0.05 to 0.5 ml/hr.

The device above, wherein the infusion tube [103] or the indwelling tube [109] is a PEGA Line 100 SF 100 cm with a 0.2 μm sterile filter, or a 200 cm infusion line with a 0.2 μm sterile filter, or a Port-a-Cath (#21-4034-24) with 22 G Needle (#21-2737-24) and extension (#21-7106-24), or other tube with similar specifications.

The device above, wherein the fluid pharmaceutical composition comprises an agent for inhibiting or suppressing expression of TGF-β, which can be used for treating or ameliorating the symptoms of a CNS disease in a human subject or animal.

The device above, wherein the fluid pharmaceutical composition comprises microparticles or nanoparticles of an agent, medicament, or delivery vehicle.

The device above, wherein the fluid pharmaceutical composition is therapeutic for CNS disease or CNS cancer.

The device above, wherein the fluid pharmaceutical composition is therapeutic for glioma, glioblastoma, diffuse intrinsic pontine glioma (DIPG), diffuse midline glioma (DMG), leptomeningeal or brain metastasis, brain or spinal cancer, or CNS tumors.

The device above, wherein the fluid pharmaceutical composition comprises an agent for inhibiting or suppressing expression of TGF-β2 selected from TGF-β2-specific antisense oligonucleotides complementary to a TGF-β2 transcript as follows: Table 2 SEQ ID NOs:1-136 and chemically-modified variants thereof, LNA variants thereof, gapmer variants thereof, and any combination thereof.

The device above, wherein the device is operated in combination with radiation therapy, or electric field therapy.

A device for delivering a pharmaceutical composition by intracranial continuous infusion, the device comprising:
a reservoir [402] containing the pharmaceutical composition, the reservoir comprising a hard shell [403], a flexible top [401], and a non-flexible mounting plate [405];
a port [407] in fluid communication with the reservoir; and
an entry catheter [413] in fluid communication with the reservoir, wherein the entry catheter is substantially linear and enters intraventricularly into a target region of the brain.

A device for delivering a pharmaceutical composition by intracranial continuous infusion, the device comprising:
a reservoir [502] containing the pharmaceutical composition, the reservoir comprising an upper hard shell [503], a lower hard shell [504], a flexible top [501], and a non-flexible mounting plate [505];
a port [507] in fluid communication with the reservoir for attaching an infusion line; and
a port [509] in fluid communication with the reservoir for attaching an entry catheter.

A device for delivering a pharmaceutical composition by intracranial continuous infusion, the device comprising:
a reservoir [602] containing the pharmaceutical composition, the reservoir comprising an upper hard shell [603], a flexible top [601], and a non-flexible mounting plate [605];
a port [619] in fluid communication with the reservoir for attaching an infusion line; and
a port [607] in fluid communication with the reservoir for attaching an entry catheter.

A collar for a device for delivering a pharmaceutical composition by intracranial continuous infusion, the collar comprising:

a hard shell [604] having an opening [606] which exposes a flexible top of the device.

A method for administering a pharmaceutical composition by intracranial continuous infusion, comprising:
mounting a device according to above to a patient; and
pumping the pharmaceutical composition in the device to provide intracranial continuous infusion to the patient.

The method above, wherein the device is mounted and the entry catheter is placed into the brain without concurrent head or brain imaging.

The method above, wherein the device is mounted and a single entry catheter is placed into the brain.

A kit for continuous intracranial infusion of a pharmaceutical composition into a subject, the kit comprising:
a reservoir containing the pharmaceutical composition;
a pump;
an ommaya reservoir having a partially-flexible top;
infusion tubes for connecting the reservoir to the pump, and connecting the pump to the ommaya reservoir;
a filter; and
an entry catheter.

The kit above, wherein the entry catheter is substantially linear for intraventricular entry into a target region of the brain.

The kit above, wherein the entry catheter is non-linear and has a bend for intraventricular entry into a target region of the brain.

The kit above, wherein the infusion tubes are external to the subject.

The kit above, wherein a portion of the infusion tube connecting the pump to the ommaya reservoir is indwelling the subject.

### BRIEF DESCRIPTION OF THE DRAWINGS

FIG. 1 shows a device for delivering a pharmaceutical composition by intrathecal or intraventricular continuous infusion. The device shown in FIG. 1 includes an external portion which advantageously allows the pump [101] and reservoir [117] to be located or attached external to the patient's body, or anywhere on the patient's body. The external portion delivers the pharmaceutical composition through an infusion tube [103] from the pump and forces it into an ommaya reservoir [111]. The device for delivering a pharmaceutical composition by intrathecal or intraventricular continuous infusion comprises a reservoir [117] containing the pharmaceutical composition, a pump [101] to force the pharmaceutical composition through an infusion tube [103] into an ommaya reservoir [111], the pump being in fluid communication with the ommaya reservoir, wherein the pump is in fluid communication with the reservoir through a reservoir tube [115], a filter [105] in line with the infusion tube, and an entry catheter [113] in fluid communication with the ommaya reservoir, wherein the entry catheter is substantially linear and enters intraventricularly into a target region of the brain.

FIG. 2 shows a device for delivering a pharmaceutical composition by intrathecal or intraventricular continuous infusion. The device shown in FIG. 2 includes an indwelling portion which advantageously allows portability of the device by the patient. The indwelling portion includes an indwelling tube [109] in fluid communication with an access port [107] and the ommaya reservoir [111], the indwelling tube to be used to transfer fluid from the access port to the ommaya reservoir. The device for delivering a pharmaceutical composition by intrathecal or intraventricular continuous infusion comprises a reservoir [117] containing the pharmaceutical composition, a pump [101] to force the pharmaceutical composition through a pumping tube [103] into an access port [107], the pump being in fluid communication with the ommaya reservoir, wherein the reservoir is in fluid communication with the pump through a reservoir tube [115], a filter [105] in line with the pumping tube, an indwelling tube [109] fluidly connected from the access port to the ommaya reservoir [111], and an entry catheter [113] in fluid communication with the ommaya reservoir, wherein the entry catheter is substantially linear and enters intraventricularly into a target region of the brain.

FIG. 3 shows a device for delivering a pharmaceutical composition by intrathecal or intraventricular continuous infusion. The device shown in FIG. 3 includes an external portion which advantageously allows the pump [101] and reservoir [117] to be located or attached external to the patient's body, or anywhere on the patient's body. The external portion delivers the pharmaceutical composition through an infusion tube [103] from the pump and forces it into an ommaya reservoir [111]. The device for delivering a pharmaceutical composition by intrathecal or intraventricular continuous infusion comprises a reservoir [117] containing the pharmaceutical composition, a pump [101] to force the pharmaceutical composition through an infusion tube [103] into an ommaya reservoir [111], the pump being in fluid communication with the ommaya reservoir, wherein the pump is in fluid communication with the reservoir through a reservoir tube [115], a filter [105] in line with the infusion tube, and an entry catheter [113] in fluid communication with the ommaya reservoir, wherein the entry catheter is non-linear and has a bend to enter intraventricularly into a target region of the brain.

FIG. 4 shows a device for delivering a pharmaceutical composition by intrathecal or intraventricular continuous infusion. The device shown in FIG. 4 includes an indwelling portion which advantageously allows portability of the device by the patient. The indwelling portion includes an indwelling tube [109] in fluid communication with an access port [107] and the ommaya reservoir [111], the indwelling tube to be used to transfer fluid from the access port to the ommaya reservoir. The device for delivering a pharmaceutical composition by intrathecal or intraventricular continuous infusion comprises a reservoir [117] containing the pharmaceutical composition, a pump [101] to force the pharmaceutical composition through a pumping tube [103] into an access port [107], the pump being in fluid communication with the ommaya reservoir, wherein the reservoir is in fluid communication with the pump through a reservoir tube [115], a filter [105] in line with the pumping tube, an indwelling tube [109] fluidly connected from the access port to the ommaya reservoir [111], and an entry catheter [113] in fluid communication with the ommaya reservoir, wherein the entry catheter is non-linear and has a bend to enter intraventricularly into a target region of the brain.

FIG. 5 shows an ommaya delivery device for use in a method for delivering a pharmaceutical composition by intrathecal or intraventricular continuous infusion. The ommaya delivery device shown in FIG. 5 can be used for example as the ommaya reservoir [111] in FIGS. 1-2. The ommaya delivery device includes a non-flexible hard shell [403] composed of an inert material such as metal or hard plastic, which encloses and defines a reservoir [402] for a drug-containing fluid. The ommaya delivery device also includes a flexible top [401] composed of a flexible material such as rubber, elastomer, or plastic. The area of the flexible top [401] relative to the entire shell ([401] plus [403]) can be from 10% flexible to 50% flexible. The ommaya delivery device further includes a non-flexible mounting plate [405] composed of an inert material such as metal or hard plastic. The ommaya delivery device further includes port [407] for connecting infusion lines to be in fluid communication with the ommaya delivery device. For example, port [407] can connect to infusion tube [103] in FIGS. 1-2. Fluid communication between the reservoir defined by the shell [403] and the infusion lines is provided by the interior channel [421]. The ommaya delivery device further includes an entry catheter [413] in fluid communication with the ommaya reservoir, wherein the entry catheter is substantially linear and enters intraventricularly into a target region of the brain. Fluid communication between the reservoir defined by the shell [403] and the entry catheter is provided by the interior channel [423]. In another embodiment, hard shell [403] can be a separate protective collar that can be placed over a completely flexible top having the entire area of [401] plus [403] to reduce the exposed area of the flexible top to the area of [401] (see FIG. 8).

FIG. 6 shows an ommaya delivery device for use in a method for delivering a pharmaceutical composition by intrathecal or intraventricular continuous infusion. The ommaya delivery device shown in FIG. 6 can be used for example as the ommaya reservoir [111] in FIGS. 1-2. The ommaya delivery device includes a non-flexible hard shell [503] and [504] composed of an inert material such as metal or hard plastic, which encloses and defines a reservoir [502] for a drug-containing fluid. The ommaya delivery device also includes a flexible top [501] composed of a flexible material such as rubber, elastomer, or plastic. The area of the flexible top [501] relative to the entire shell ([501] plus [503]) can be from 10% flexible to 50% flexible, as shown in the lower view of FIG. 6 (view A-A). The ommaya delivery device further includes a non-flexible mounting plate [505] composed of an inert material such as metal or hard plastic. The ommaya delivery device further includes ports [507] and [509] for connecting infusion lines to be in fluid communication with the ommaya delivery device. For example, port [507] can connect to indwelling tube [109] in FIGS. 3-4. Fluid communication between the reservoir defined by the shell [503] and the infusion lines is provided by the interior channel [521]. For example, port [509] can connect to entry catheter [113] in FIGS. 1-2. In another embodiment, hard shell [503] can be a separate protective collar that can be placed over a completely flexible top having the entire area of [501] plus [503] to reduce the exposed area of the flexible top to the area of [501] (see FIG. 8).

FIG. 7 shows an ommaya delivery device for use in a method for delivering a pharmaceutical composition by intrathecal or intraventricular continuous infusion. The ommaya delivery device shown in FIG. 7 can be used for example as the ommaya reservoir [111] in FIGS. 3-4. The ommaya delivery device includes a non-flexible hard shell [603] composed of an inert material such as metal or hard plastic, which encloses and defines a reservoir [602] for a drug containing fluid. The ommaya delivery device also includes a flexible top [601] composed of a flexible material such as rubber, elastomer, or plastic. The ommaya delivery device further includes a non-flexible mounting plate composed of an inert material such as metal or hard plastic. The ommaya delivery device further includes ports [607] and [619] for connecting infusion lines to be in fluid communication with the ommaya delivery device. For example, port [607] can connect to infusion tube [103] in FIGS. 3-4. Fluid communication between the reservoir defined by the shell [603] and the infusion lines is provided by the interior channel [621]. For example, port [619] can connect to entry catheter [113] in FIGS. 3-4. Fluid communication between the reservoir defined by the shell [603] and the entry catheter is provided by the interior channel [619]. In another embodiment, hard shell [603] can be a separate protective collar that can be placed over a completely flexible top having the entire area of [601] plus [603] to reduce the exposed area of the flexible top to the area of [601] (see FIG. 8).

FIG. 8 shows an embodiment of a collar for an ommaya delivery device for use in a method for delivering a pharmaceutical composition by intrathecal or intraventricular continuous infusion. In this embodiment, hard shell [604] is a separate protective collar that can be placed over a completely flexible top of an ommaya reservoir to make the top partially flexible. The opening [606] exposes only a portion of the flexible top of the ommaya reservoir.

FIG. 9 shows the effect of delivering a pharmaceutical composition by intrathecal or intraventricular continuous infusion. FIG. 9 shows the effect of OT-101 Treatment on TGF-β2 Secretion from the Human GBM cell line A-172. Cells were incubated with the indicated different concentrations of OT-101/AP 12009 (1 μM to 80 μM) for 7 days. Secreted TGF-β2 was measured in cell supernatants by ELISA. Results represent median, minimum, and maximum values from 3 independent experiments.

FIG. 10 shows infusion from an ommaya-like reservoir of this invention having a surface area of 339 mm² and a diameter of the inner opening of the catheter of 1.4 mm. In this example, a dark colored test solution is shown slowly diffusing downward through the catheter opening. This diffusion from the ommaya-like reservoir of this invention occurred continuously over a period of hours.

FIG. 11 shows continuous infusion operating parameters can be modified to extend the drug infusion release kinetic half-life from hours to days.

FIG. 12 shows continuous infusion operating parameters can be modified to extend the drug infusion release kinetic half-life from days to weeks.

FIG. 13 shows continuous infusion operating parameters can be modified to extend the drug infusion release kinetic half-life from weeks to months.

FIG. 14 shows mRNA levels for TGFB1/2/3 isoforms in 41 primary DIPG samples vs. 29 normal pons specimens.

FIG. 15 shows statistically significant positive correlations with TGFB2 mRNA levels.

FIG. 16 shows correlated expression with specific transcription factors in tumor specimens from 41 pediatric patients with DIPG (n=29) or H3K27M-mutant GBM.

FIG. 17 shows TGFB2-high DIPG patients exhibited a significantly worse OS outcome than TGFB2low DIPG patients.

FIG. 18 shows PFS outcome was also significantly worse for the TGFB2-high subset.

FIG. 19 shows patients in the TGFB2-high (N=29) and TGFB2-low (N=87) subsets exhibited very similar OS outcomes.

FIG. 20 shows expression is selectively amplified and associated with poor OS in pediatric DIPG.

FIG. 21 shows a waterfall plot depicting the maximum log 10 reduction values for tumor volumes in High-Grade Glioma Patients Treated with OT-101 Monotherapy Who Achieved a CR or PR.

FIG. 22 shows a semi-log plot of the combined 3-D tumor volume reduction curve for the 19 High-Grade Glioma Patients Treated with OT-101 Monotherapy Who Achieved a CR or PR.

FIG. 23 shows a swimmer plot for onset and duration of objective response for High-Grade Glioma Patients Treated with OT-101 Monotherapy Who Achieved a CR or PR. The onset and duration of CR/PR, end of OR and onset of PD are indicated with specific signals.

FIG. 24 shows superimposed Kaplan-Meier survival curves of both OT-101 and standard chemotherapy.

FIG. 25 shows chemotherapy-naive treated with temozolomide (TMZ).

FIG. 26 shows chemotherapy-failure treated with chemotherapy agents CCNU/BCNU.

FIG. 27 shows TGF-β2 was a valid target for therapy against gliomas using three TGFB2 probesets which exhibited increased levels of expression in DIPG patients.

FIG. 28 shows TGF-β2 was a valid target for therapy against pediatric GBM using TGFB2 probesets which exhibited increased levels of expression in DIPG patients.

FIG. 29 shows that for gliomas in patients treated with radiation there was an Overall Survival advantage with low TGF-β2 expression. Only TGF-β2 was predictive of survival.

FIG. 30 shows that for gliomas in patients treated with radiation there was an Overall Survival advantage with low TGF-β2 expression. Only TGF-β2 was predictive of survival.

FIG. 31 shows TGF-β2 level is selectively predictive of improved overall survival (OS) in combination with chemotherapy (TMZ).

FIG. 32 shows TGF-β2 level is selectively predictive of improved overall survival (OS) in combination with chemotherapy TMZ and radiation.

FIG. 33 shows TGF-β2 level is selectively predictive of improved overall survival (OS) in combination with antiangiogenic therapy (bevacizumab).

### DETAILED DESCRIPTION OF THE DISCLOSURE

This invention provides a novel device and methods of use for delivery and administration of therapeutic agents ranging from bolus administration to fixed rate infusion and continuous infusion. The devices and methods of this invention can be used to treat CNS diseases such as CNS cancers through intracranial routes.

Devices and methods of this invention can deliver agents to the brain or spinal regions using a pumped, continuous infusion device/system.

This invention provides therapies for treating or ameliorating symptoms of CNS disease such as CNS cancer.

In some embodiments, this invention includes agents and compositions for inhibiting or suppressing TGF-β2 to provide improved clinical outcomes for CNS disease.

In further embodiments, this invention provides stable formulations of anti-TGF-β2 agents for various therapies for CNS disease. Examples of anti-TGF-β2 agents include TGF-β inhibitors such as antisense oligonucleotides, pharmaceutically acceptable salts forms, esters, polymorphs or stereoisomers thereof, as well as combinations thereof.

In further aspects, this disclosure provides highly stable formulations of anti-TGF-β2 agents for therapies for CNS disease. The stable formulations of this invention provide surprisingly improved clinical results. Stable formulations of agents for suppressing TGF-β can be used to treat CNS disease, in particular CNS cancer.

This invention further provides a novel device for continuous infusion operating parameters can be modified to extend the drug infusion release kinetic half-life from days to weeks of agents to treat CNS diseases such as CNS cancers through intrathecal or intraventricular routes.

Devices and methods of this invention can deliver agents to the brain or spinal regions using a pumped, continuous infusion system.

In some embodiments, this invention provides an ommaya reservoir device with catheter access to cerebrospinal fluid and intraventricular spaces which is connected to a pump for increased and continuous infusion.

In some embodiments, this invention provides an ommaya-like reservoir device with catheter access to cerebrospinal fluid and intraventricular spaces which is connected to a pump for increased and continuous infusion.

This invention provides a device for enhanced delivery, in some circumstances a portable delivery device, that provides delivery by infusion of liquids to specific locations within the body, especially brain tissues and tumors, preferably the ventricular space.

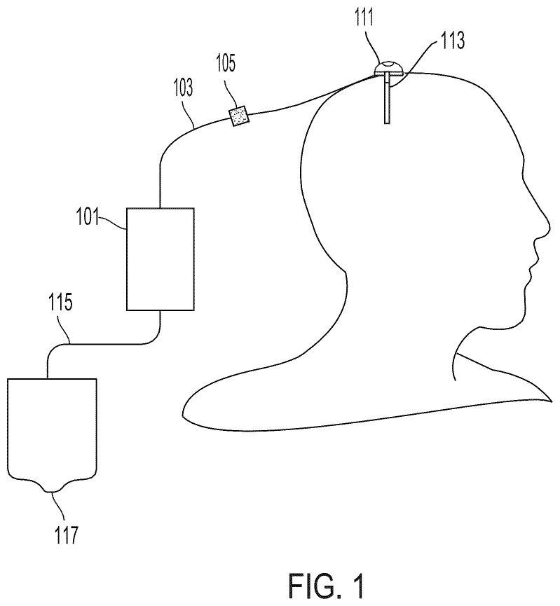

This invention also provides a device for intrathecal or intraventricular continuous infusion of therapeutics which includes an ommaya-like device (see FIGS. 1-8 and 10). The ommaya-like device allows access of an entry catheter into a ventricle of the brain. The entry catheter may alternatively reach intraventricularly to a brain tumor, or other part of the brain, for delivery of therapeutics. The ommaya-like device also acts advantageously as a reservoir to hold the therapeutics for prolonged and sustained release into the underlying brain matter or ventricle. The ommaya-like device can be made of a non-collapsible material, and have a portion that is flexible (see FIGS. 1-7). This structure and design may create oscillating compression and decompression to maintain fluid movement in and out of the ommaya-like device reservoir. In effect, the novel the ommaya-like device and device of this invention may advantageously allow a breathing of the reservoir. In operative use, the ommaya-like device and method may include self pulsating or self oscillating operation due to physical and muscular activities of the patient that stretch and release the skin near the reservoir and maintain fluid movement and drug release. The flexible top enhances fluid movement in the chamber or internal reservoir. Without the flexible top a stagnant compartment may not allow the fluid drug composition to flow out adequately for patient infusion.

The flexible top may enhance fluid movement by acting as a shock arrestor to create more uniform fluid flow rate in the catheter.

In some aspects, the ommaya-like device and device of this invention may include a pump for continuous pumping to drive fluid flow in and out of the ommaya-like device reservoir.

This invention provides a device for therapies for treating or ameliorating symptoms of CNS disease such as CNS cancer.

In some aspects, this invention describes a device using a pump compatible with infusion of agents and compositions for inhibiting or suppressing TGF-β2 to provide improved clinical outcomes for CNS disease.

In further aspects, this invention provides stable formulations of anti-TGF-β2 agents for various therapies for CNS disease, where the formulations can be pumped in continuous infusion. Examples of anti-TGF-β2 agents include TGF-β inhibitors such as antisense oligonucleotides, modifications such as LNA/2-MOE, pharmaceutically acceptable salts forms, esters, polymorphs or stereoisomers thereof, as well as combinations thereof.

The device of this invention links an appropriate pump to deliver small amounts of a pharmaceutical composition continuously over a period of days.

The device of this invention has the advantage of using an ommaya intraventricular catheter to deliver a pharmaceutical composition in long term infusion, either with externally mounted infusion lines, or with an indwelling infusion architecture.

In another example, the device of this invention can use an ommaya reservoir catheter as an intratumoral catheter to deliver a pharmaceutical composition. This device has the advantage to be used with an external pump for short period infusions, or with extended internal tubing and catheters that are tunneled under the skin for long term placement for infusion. The ommaya reservoir component of the device of this invention may provide long term access to cerebrospinal fluid and intraventricular spaces.

The infusion device of this invention also has the advantage to deliver a pharmaceutical composition to a tumor at any location in the brain with easy insertion of the catheter from the ommaya reservoir into the intraventricular space which does not require insertion under imaging.

The infusion device of this invention also has the advantage of using one standardized intraventricular target.

The infusion device of this invention also has the advantage that the intraventricular path to the target is well understood and safe to use.

The infusion device of this invention also has the advantage that effective delivery of the pharmaceutical composition can be done to the entire CNS, including the spinal cord.

The infusion device of this invention also has the advantage that it does not require imaging such as x-ray imaging to guide implantation of the delivery catheter. Because the device acts as a tap, when the catheter tip reaches the CSF fluid, the CSF fluid will backflow to indicate successful placement of the catheter.

In some aspects, the infusion device of this invention also has the advantage to be used with placement of the entry catheter tip to a lesser distance into the patient. The entry catheter can be shorter, and used in a shallower placement because the device takes advantage of natural fluid circulation in the patient, for example in the brain.

In further aspects, the infusion device of this invention also has the advantage to be used with placement of a single entry catheter to provide infusion into the patient. For example, treatment of glioma or large brain tumors can require the placement of several catheters near the cancer. However, the device of this invention can provide improved infusion with even a single catheter near the tumor.

The infusion device of this invention also has the advantage that the implantation of the delivery catheter can be semi-permanent, or even permanent.

The infusion device of this invention also has the advantage that the ommaya reservoir may have access for needle aspiration of fluid from the device or brain, or the injection of fluid into the device or brain.

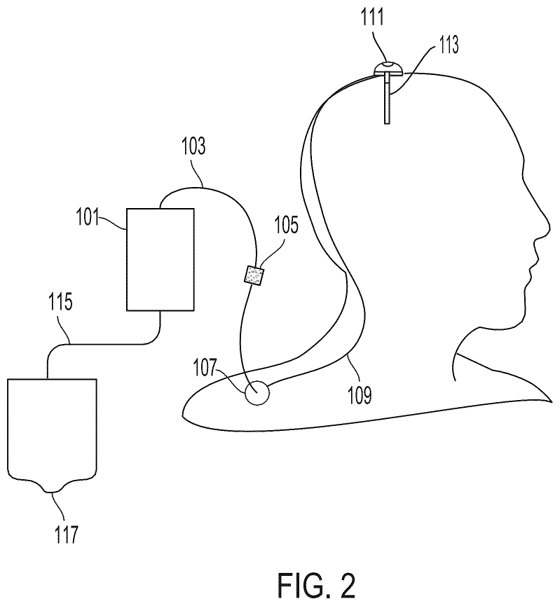

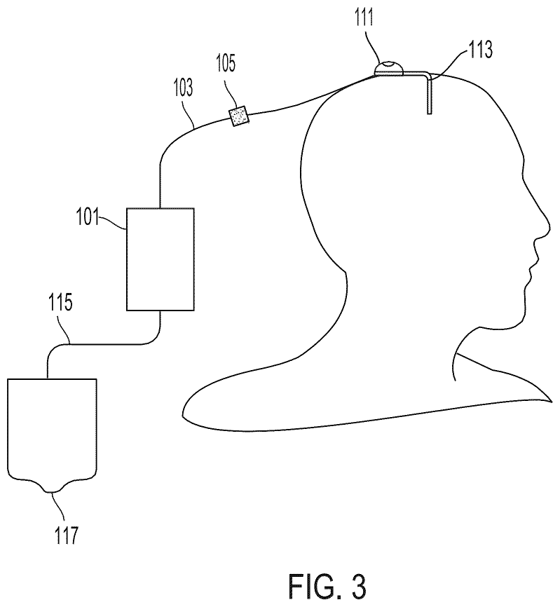

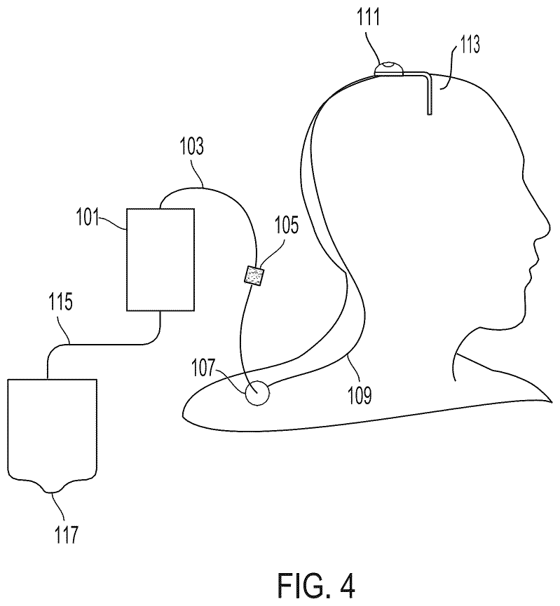

The infusion device of this invention also has the advantage that the ommaya device can be placed and utilized as needed for effective delivery of drug and treatment. For example, the ommaya device can be placed directly on top of the ventricles of the brain and employ a straight ventricular entry catheter [113] as in FIGS. 1 and 2. In a further example, the ommaya device can be placed in a convenient position on the skull of the patient and connected to a ventricular entry catheter [113] that is bent at an angle as in FIGS. 3 and 4 to reach a target position in the brain.

Aspects of this invention include the following:

A device for delivering a pharmaceutical composition by intrathecal or intraventricular continuous infusion, the device comprising (FIG. 1): a reservoir [117] containing the pharmaceutical composition; a pump [101] to force the pharmaceutical composition through an infusion tube [103] into an ommaya reservoir [111], the pump being in fluid communication with the ommaya reservoir, and wherein the pump is in fluid communication with the reservoir through a reservoir tube [115]; a filter [105] in line with the infusion tube; and an entry catheter [113] in fluid communication with the ommaya reservoir, wherein the entry catheter is substantially linear and enters intraventricularly into a target region of the brain. The entry catheter [113] may be non-linear and has a bend to enter intraventricularly into a target region of the brain (FIG. 3).

A device for delivering a pharmaceutical composition by intrathecal or intraventricular continuous infusion, the device comprising (FIG. 2): a reservoir [117] containing the pharmaceutical composition; a pump [101] to force the pharmaceutical composition through a pumping tube [103] into an access port [107], the pump being in fluid communication with the ommaya reservoir, wherein the reservoir is in fluid communication with the pump through a reservoir tube [115]; a filter [105] in line with the pumping tube; an indwelling tube [109] in fluid communication with the access port and the ommaya reservoir [111]; and an entry catheter [113] in fluid communication with the ommaya reservoir, wherein the entry catheter is substantially linear and enters intraventricularly into a target region of the brain. The entry catheter [113] may be non-linear and have a bend to enter intraventricularly into a target region of the brain (FIG. 4).

The device can provide continuous infusion of a therapeutically effective amount of the pharmaceutical composition to the target region.

As used herein, a target region of the brain may be a region containing a tumor.

The distal end of the entry catheter enters the target region of the brain.

The ommaya reservoir may hold the pharmaceutical composition behind a membrane for a sustained period of time for sustained release of the pharmaceutical composition to the entry catheter. The distal end tip of the entry catheter that enters the brain is a step-down end, a recessed step end, a multi-port end, a microporous end, or a balloon tipped end. In some aspects, the entry catheter may be barium-impregnated silicone and have resistance to kinking and compression. In additional aspects, the entry catheter may include a slender, surgically-acceptable probe, such as a stainless steel probe, to allow the catheter to be directed during catheter placement.

The pump can be a Pegasus Vario, a PEGA PCA, a CADD Solis VIP, a CADD-Legacy PLUS, a CADD-Legacy PCA, a CADD-Legacy 1, or other pumps with similar specifications.

The infusion rate may be from 0.01 to 3000 ml/hr, or from 0.01 to 100 ml/hr, or from 0.01 to 2 ml/hr, or from 0.01 to 1 ml/hr, or from 0.05 to 0.5 ml/hr.

Some examples of a pump and infusion rates for the device of this invention include the following in Table 1:

TABLE 1
Pumps and rates for infusion system

| Pumps | Manufacturer | Infusion Rate | |
|---|---|---|---|
| External | | | |
| Pegasus Vario | LogoMed | 0.01-15 | ml/hr |
| PEGA PCA | Venner Medical | 0.1-100 | ml/h |
| CADD Solis VIP | Smiths Medical | 0.1-500 | ml/hr |
| CADD-Legacy | Smiths Medical | 0.1-125 | ml/hr |
| PLUS | | | |
| CADD-Legacy PCA | Smiths Medical | 0-50 | ml/hr |
| Intrathecal/CSF | | | |
| Synchromed II | Medtronic | 0.048-24 | ml/day |
| Prometra | Flowonix | 0-28.8 | mL/day |

The infusion tube [103] or the indwelling tube [109] may be a PEGA Line 100 SF 100 cm with a 0.2 um sterile filter, or a 200 cm infusion line with a 0.2 um sterile filter, or a Port-a-Cath (#21-4034-24) with 22 G Needle (#21-2737-24) and extension (#21-7106-24), or other tubes with similar specifications.

In some aspects, the brain entry catheter positioning into the ventricular space can be a nonspecific ventricular catheter. The ventricular catheter may have an inner diameter of from 1.0 to 2.0 mm. For example, the ventricular catheter may have an inner diameter of 1.4 mm and an outer diameter of 2.7 mm. The catheter may be 14 cm in length, or shorter, and can be supplied with 24 inlet holes (for example, 3 rows of 8 holes) at the proximal end. In general, the inner diameter of the catheter may dictate the diffusion of the drug into the ventricular space. The ommaya-like reservoir of this invention may have a surface area of from 300 to 400 mm², and the area of the inner opening of the catheter can be from 0.785 mm² to 3.14 mm². Thus the ratio of the reservoir surface area to the catheter surface area will have a range from 96 to 509. With this ratio, the expected release time of the drug solution can be adjusted over a wide range, depending on the density of the test solution to water.

The ommaya-like device of the invention allows the delivery of drugs directly into the CSF and is especially useful for delivering oligonucleotides and antisense oligonucleotide drugs such as OT-101. Either alone or in combination with other cancer therapy.

In some aspects, the drug load may be surprisingly delivered as a single bolus infusion, or as a short infusion of 15, 30, or 60 min, and still achieve sustained delivery by the advantageous ratio of reservoir volume to catheter opening and/or the adjusted ratio of CSF density to drug solution density.

For example, the usual surface area of the reservoir is 339 mm² and the inner diameter of opening of catheter is 1.4 mm. The release profile of the device can be controlled by varying the ratio of reservoir surface area versus catheter surface area as related to the diffusion equation.

As described hereinbelow, rat animal infusion data was obtained with only a ventricular catheter and no reservoir and the equation {Y=Span*exp(−K*X)+Plateau}governed the process, where normalized value may achieve a span of 100 and plateau of 0, with k of 7.571. Imposing a constraint being the drug reservoir, the change in the rate in relation to surface area ratio of reservoir to catheter was determined. Varying the reservoir to catheter ratio, the drug infusion release kinetic can be slowed such that half-life may advantageously become days and weeks.

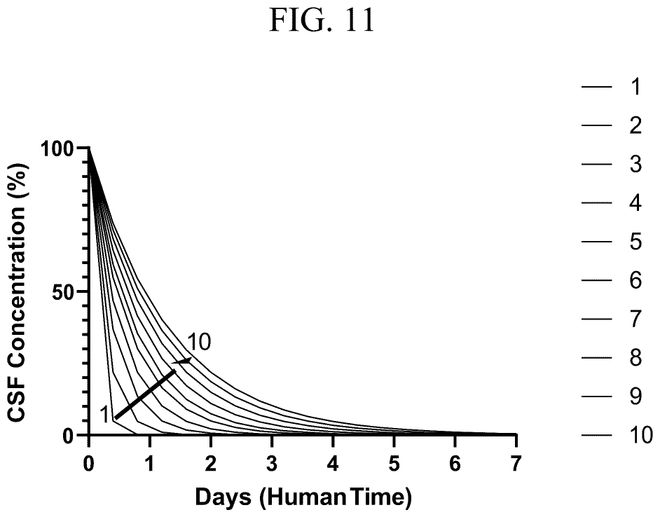

For example, as shown in FIG. 11, the drug infusion release kinetic can be extended such that the half-life changes from hours to days for a single reservoir.

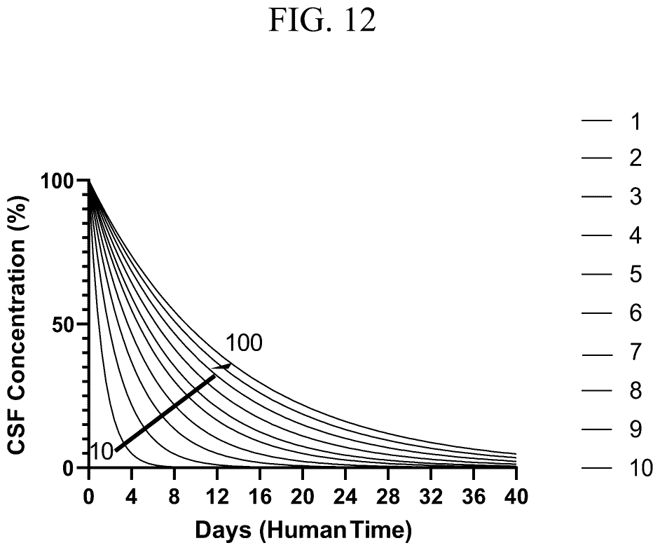

For example, as shown in FIG. 12, the drug infusion release kinetic can be extended such that the half-life changes from days to weeks for a single reservoir.

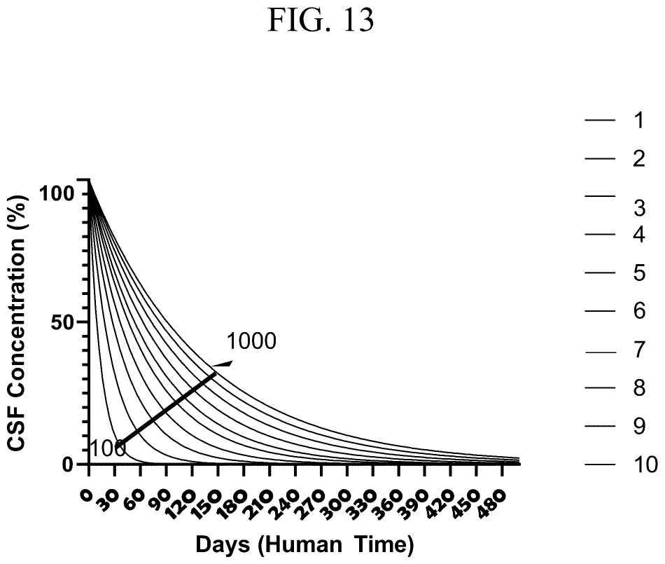

For example, as shown in FIG. 13, the drug infusion release kinetic can be extended such that the half-life changes from weeks to months for a single reservoir.

An advantage of the device of this invention is the design of the ommaya-like reservoir which allows the device to maintain fluid flow and movement into, and out of the reservoir. The flexible top expands and contracts in response to pressure changes, which allows the ommaya-like reservoir of this invention to breath properly for continuous flow of the solution, especially in the presence of externally generated pumping action. Unwanted accidental or incidental release can also occur due to patient movements, for example, movement of the patient's jaw and other muscles.

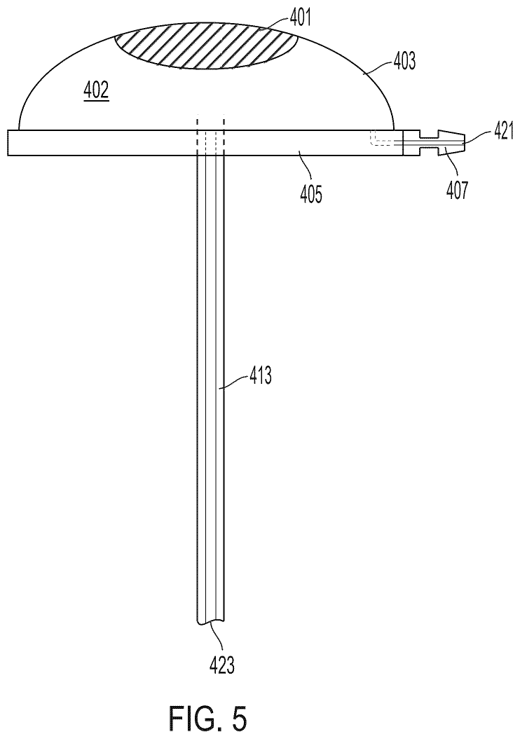

An advantage of the device of this invention is the placement of the ommaya-like device not as a mechanism to collect CSF, or for delivery of drug (pharmaceutical composition) to CSF, but as a reservoir to hold drug that will infuse into the CSF over a sustained period of time. In order for fluid to leave the ommaya, a continuous flow out of the reservoir is driven by a pump, which also brings drug fluid into the reservoir. The structure of the device of this invention advantageously prevents accidental compression of the ommaya that might deliver a large quantity of drug from the reservoir suddenly into the CSF because the ommaya delivery device portion of the device of this invention has a rigid, non-flexible hard shell and is only partially collapsible (see FIGS. 1-7). Further, the design of the device of this invention advantageously maintains fluid movement in the reservoir because the reservoir includes a flexible top (for example, see FIG. 5 [401]), which may create oscillating compression and decompression for fluid movement. For example, movement of the skin or jaw muscles may compress and decompress the ommaya device, slightly because of the flexible portion, and keep fluid moving in and out of the reservoir.

As used herein, a partially-flexible top can refer to an ommaya-like reservoir made from a hard material with an integrated top where a only an uppermost portion of the top is flexible, or an ommaya-like reservoir having a top made from a flexible material and fitted with a hard collar so that only an uppermost portion of the flexible top is exposed and can be depressed or flexed.

The pharmaceutical composition may comprise an agent for inhibiting or suppressing expression of TGF-β, which can be used for treating or ameliorating the symptoms of a CNS disease in a human subject or animal. The CNS disease may be a cancer. The cancer may be a glioma, a glioblastoma, a diffuse intrinsic pontine glioma (DIPG), a diffuse midline glioma (DMG), a leptomeningeal or brain metastasis, a brain or spinal cancer, or CNS tumors.

In some embodiments, compositions and methods of this invention can be used for diffuse midline glioma (DMG) and K27M GBM.

### Methods and Compositions for CNS Disease

Operating parameters. The system of this invention can be modified to extend the drug used for treating CNS disease such as CNS cancer or ameliorating the symptoms of CNS disease in a human subject or animal in need. The system of this invention may contain a pharmaceutical composition for continuous infusion release kinetic half-life from weeks to months to the brain or spine.

A pharmaceutical composition for continuous infusion may include an agent for inhibiting or suppressing expression of TGF-β, and administering a therapeutically sufficient amount of the composition to the subject. This invention provides a system therapies for treating CNS disease such as cancer or ameliorating symptoms of the CNS disease.

Examples of anti-TGF-β2 agents include TGF-β inhibitors such as antisense oligonucleotides, artemisinin, pharmaceutically acceptable salts forms, esters, polymorphs or stereoisomers thereof, as well as combinations thereof.

In some aspects, this invention provides a system containing a composition of an agent for inhibiting or suppressing expression of TGF-β for treating or ameliorating the symptoms of CNS disease in a human subject or animal.

In further aspects, this invention provides uses of a composition of an agent for inhibiting or suppressing expression of TGF-β in the preparation of a medicament for treating or ameliorating the symptoms of CNS disease in a human subject or animal.

As used herein, “intraventricular” infusion may be used for continuous infusion of a pharmaceutical composition to treat CNS disease including cancer. An example of intraventricular administration is an ommaya-like device with entry catheter.

As used herein, the term intracranial encompasses intrathecal and intraventricular. For example, intracranial infusion includes intrathecal infusion and intraventricular infusion. Further, the term “intrathecal and intraventricular” is intended to encompass “intracranial.”

### Human TGF-β2-Specific Phosphorothioate Antisense Oligodeoxynucleotide

An antisense oligonucleotide (ASO) can be a single-stranded deoxyribonucleotide, which may be complementary to an mRNA target. The antisense therapy may downregulate a molecular target, which may be achieved by induction of RNase H endonuclease activity that cleaves the RNA-DNA heteroduplex with a significant reduction of the target gene translation. Other ASO mechanisms can include inhibition of 5′ cap formation, alteration of splicing process such as splice-switching, and steric hindrance of ribosomal activity.

Antisense therapeutic strategies can utilize single-stranded DNA oligonucleotides that inhibit protein production by mediating the catalytic degradation of a target mRNA, or by binding to sites on mRNA needed for translation. Antisense oligonucleotides can be designed to target the viral RNA genome or viral transcripts. Antisense oligonucleotides can provide an approach for identifying potential targets, and therefore represent potential therapeutics.

Antisense oligonucleotides can be small synthetic pieces of single-stranded DNA that may be 15-30 nucleotides in length. An ASO may specifically bind to a complementary DNA/RNA sequence by Watson-Crick hybridization and once bound to the target RNA, inhibit the translational processes either by inducing cleavage mechanisms or by inhibiting mRNA maturation. An ASO may selectively inhibit gene expression with specificity. Chemical modifications of DNA or RNA can be used to increase stability.

For example, modifications can be introduced in the phosphodiester bond, the sugar ring, and the backbone. ASO antiviral agents may block translational processes either by (i) ribonuclease H (RNAse H) or RNase P mediated cleavage of mRNA or (ii) by sterically (non-bonding) blocking enzymes that are involved in the target gene translation. Human TGF-β2-specific phosphorothioate antisense oligodeoxynucleotide (OT-101; AP 12009; Trabedersen), hereafter referred to as OT-101 or AP 12009, is intended to reduce the level of TGF-β2 protein in malignant gliomas, and thereby delay the progression of disease.

Antisense oligodeoxynucleotides are short strings of DNA that are designed to downregulate gene expression by interfering with the translation of a specific encoded protein at the mRNA level. OT-101 is a synthetic 18-mer phosphorothioate oligodeoxynucleotide (S-ODN) where all 3′-5′ linkages are modified to phosphorothioates. The molecular formula is C₁₇₇H₂₀₈N₆₀Na₁₇O₉₄P₁₇S₁₇ and the molecular weight 6,143 g/mol. OT-101 was designed to be complementary to a specific sequence of human TGF-β2 mRNA following expression of the gene.

OT-101 may be supplied as a lyophilized powder in 50-mL glass vials in three different quantities. OT-101 lyophilized powder is dissolved in isotonic (0.9%) aqueous sodium chloride prior to use. The product can be prepared for administration at a desired concentration.

OT-101: Antisense oligodeoxynucleotides are short strings of DNA that are designed to downregulate gene expression by interfering with the translation of a specific encoded protein at the mRNA level. Several RNA therapeutics, including anti-sense oligonucleotides have been evaluated in clinical trials and some approved. OT-101 is a synthetic 18-mer phosphorothioate oligodeoxynucleotide (S-ODN) in which a non-bridging oxygen of each phosphate moiety is substituted by a sulfur atom. OT-101 was designed to be complementary to a specific sequence of human TGF-β2 mRNA following expression of the gene. It is a first-in-class RNA therapeutic designed to abrogate the immunosuppressive actions of TGF-β2 and reduce the level of TGF-β2 in malignant gliomas, and thereby delay the progression of disease.

The agent may be an antisense oligonucleotide or inhibitor specific for TGF-β 1, TGF-β2, or TGF-β3. The agent for inhibiting or suppressing expression of TGF-β may be selected from TGF-β2-specific antisense oligonucleotides as set forth below.

A target TGF-β2 mRNA can be NCBI Reference Sequence: NM_003238.3 of sequence length 5,882 bp. A target region for TGF-β2 mRNA can be the protein coding sequence from reference 1,369 to 2,613.

Examples of agents of this disclosure for inhibiting or suppressing expression of TGF-β2 include TGF-β2-specific antisense oligonucleotides given in SEQ ID NOs:1-136 in Table 2.

TABLE 2
TGF-β2-specific antisense oligonucleotides

| SEQ ID | | |
|---|---|---|
| NO: | Ref. | ANTISENSE SEQUENCE |
| 1 | — | GTTCGTTTAG AGAACAGATC |
| 2 | — | TAAAGTTCGT TTAGAGAACA G |
| 3 | — | AGCCCTGTAT ACGAC |
| 4 | — | GTAGGTAAAA ACCTAATAT |
| 5 | — | CGTTTAGAGA ACAGATCTAC |
| 6 | — | CATTGTAGAT GTCAAAAGCC |
| 7 | — | CTCCCTCATG GTGGCAGTTG A |
| 8 | — | CGGCATGTCT ATTTTGTA |
| 9 | 178-195 | TTTGTTCCTG GATGACTC |
| 10 | 179-196 | GTTTGTTCCT GGATGACT |
| 11 | 180-197 | AGTTTGTTCC TGGATGAC |
| 12 | 181-198 | CAGTTTGTTC CTGGATGA |
| 13 | 182-199 | TCAGTTTGTT CCTGGATG |
| 14 | 183-200 | CTCAGTTTGT TCCTGGAT |
| 15 | 716-733 | TGTGTGTGTG TGCGTGTG |
| 16 | 717-734 | GTGTGTGTGT GTGCGTGT |
| 17 | 718-735 | TGTGTGTGTG TGTGCGTG |
| 18 | 719-736 | GTGTGTGTGT GTGTGCGT |
| 19 | 720-737 | TGTGTGTGTG TGTGTGCG |
| 20 | 721-738 | GTGTGTGTGT GTGTGTGC |
| 21 | 722-739 | TGTGTGTGTG TGTGTGTG |
| 22 | 723-740 | GTGTGTGTGT GTGTGTGT |
| 23 | 724-741 | TGTGTGTGTG TGTGTGTG |
| 24 | 725-742 | GTGTGTGTGT GTGTGTGT |
| 25 | 726-743 | TGTGTGTGTG TGTGTGTG |
| 26 | 727-744 | GTGTGTGTGT GTGTGTGT |
| 27 | 728-745 | TGTGTGTGTG TGTGTGTG |
| 28 | 729-746 | GTGTGTGTGT GTGTGTGT |
| 29 | 730-747 | CGTGTGTGTG TGTGTGTG |
| 30 | 731-748 | GCGTGTGTGT GTGTGTGT |
| 31 | 732-749 | TGCGTGTGTG TGTGTGTG |
| 32 | 733-750 | GTGCGTGTGT GTGTGTGT |
| 33 | 971-988 | AGTGGCGGAT CTGAACTC |
| 34 | 972-989 | GAGTGGCGGA TCTGAACT |
| 35 | 1430-1447 | GAGTGTGCTG CAGGTAGA |
| 36 | 1432-1449 | TCGAGTGTGC TGCAGGTA |
| 37 | 1433-1450 | ATCGAGTGTG CTGCAGGT |
| 38 | 1570-1587 | GTGCTGTTGT AGATGGAA |
| 39 | 1571-1588 | GGTGCTGTTG TAGATGGA |
| 40 | 1572-1589 | TGGTGCTGTT GTAGATGG |
| 41 | 1573-1590 | CTGGTGCTGT TGTAGATG |
| 42 | 1574-1591 | CCTGGTGCTG TTGTAGAT |
| 43 | 1575-1592 | CCCTGGTGCT GTTGTAGA |
| 44 | 1576-1593 | TCCCTGGTGC TGTTGTAG |
| 45 | 1577-1594 | GTCCCTGGTG CTGTTGTA |
| 46 | 1578-1595 | AGTCCCTGGT GCTGTTGT |
| 47 | 1895-1912 | CTGGGTTGGA GATGTTAA |
| 48 | 1896-1913 | GCTGGGTTGG AGATGTTA |
| 49 | 1897-1914 | CGCTGGGTTG GAGATGTT |
| 50 | 1900-1917 | TAGCGCTGGG TTGGAGAT |
| 51 | 1903-1920 | ATGTAGCGCT GGGTTGGA |
| 52 | 1945-1962 | AGCCATTCGC CTTCTGCT |
| 53 | 1994-2011 | GTCTTTATGG TGAAGCCA |
| 54 | 1995-2012 | TGTCTTTATG GTGAAGCC |
| 55 | 1996-2013 | CTGTCTTTAT GGTGAAGC |
| 56 | 1997-2014 | CCTGTCTTTA TGGTGAAG |
| 57 | 2000-2017 | GTTCCTGTCT TTATGGTG |
| 58 | 2001-2018 | GGTTCCTGTC TTTATGGT |
| 59 | 2002-2019 | AGGTTCCTGT CTTTATGG |
| 60 | 2003-2020 | CAGGTTCCTG TCTTTATG |
| 61 | 2004-2021 | CCAGGTTCCT GTCTTTAT |
| 62 | 2183-2200 | GGTCTTCCCA CTGTTTTT |
| 63 | 2194-2211 | AGGAGATGTG GGGTCTTC |
| 64 | 2195-2212 | CAGGAGATGT GGGGTCTT |
| 65 | 2241-2258 | GGTTGGTCTG TTGTGACT |
| 66 | 2242-2259 | CGGTTGGTCT GTTGTGAC |
| 67 | 2243-2260 | CCGGTTGGTC TGTTGTGA |
| 68 | 2522-2539 | GAGAATGGTT AGAGGTTC |
| 69 | 2525-2542 | GTAGAGAATG GTTAGAGG |
| 70 | 2542-2559 | GGTGTTTTGC CAATGTAG |
| 71 | 2543-2560 | GGGTGTTTTG CCAATGTA |
| 72 | 2544-2561 | TGGGTGTTTT GCCAATGT |
| 73 | 2666-2683 | CATCATCGTT GTCGTCGT |
| 74 | 2979-2996 | GAACGGTACG TACAGCAA |
| 75 | 2980-2997 | GGAACGGTAC GTACAGCA |
| 76 | 2981-2998 | AGGAACGGTA CGTACAGC |
| 77 | 2982-2999 | TAGGAACGGT ACGTACAG |
| 78 | 2983-3000 | ATAGGAACGG TACGTACA |
| 79 | 2984-3001 | GATAGGAACG GTACGTAC |
| 80 | 2985-3002 | GGATAGGAAC GGTACGTA |
| 81 | 2986-3003 | GGGATAGGAA CGGTACGT |
| 82 | 3029-3046 | GGGTGCCTAT TGCATAGC |
| 83 | 3030-3047 | AGGGTGCCTA TTGCATAG |
| 84 | 3031-3048 | AAGGGTGCCT ATTGCATA |
| 85 | 3032-3049 | GAAGGGTGCC TATTGCAT |
| 86 | 3033-3050 | GGAAGGGTGC CTATTGCA |
| 87 | 3035-3052 | TGGGAAGGGT GCCTATTG |
| 88 | 3036-3053 | ATGGGAAGGG TGCCTATT |
| 89 | 3037-3054 | AATGGGAAGG GTGCCTAT |
| 90 | 3038-3055 | GAATGGGAAG GGTGCCTA |
| 91 | 3039-3056 | AGAATGGGAA GGGTGCCT |
| 92 | 3040-3057 | AAGAATGGGA AGGGTGCC |
| 93 | 3041-3058 | TAAGAATGGG AAGGGTGC |
| 94 | 3042-3059 | GTAAGAATGG GAAGGGTG |
| 95 | 3043-3060 | AGTAAGAATG GGAAGGGT |
| 96 | 3044-3061 | GAGTAAGAAT GGGAAGGG |
| 97 | 3259-3276 | CAGACTTTCT CGGTCATA |
| 98 | 3260-3277 | GCAGACTTTC TCGGTCAT |
| 99 | 3261-3278 | TGCAGACTTT CTCGGTCA |
| 100 | 3262-3279 | ATGCAGACTT TCTCGGTC |
| 101 | 3263-3280 | AATGCAGACT TTCTCGGT |
| 102 | 3264-3281 | TAATGCAGAC TTTCTCGG |
| 103 | 4281-4298 | GACCTGGACT TTTTTCCC |
| 104 | 4282-4299 | TGACCTGGAC TTTTTTCC |
| 105 | 4283-4300 | CTGACCTGGA CTTTTTTC |
| 106 | 4284-4301 | GCTGACCTGG ACTTTTTT |
| 107 | 4467-4484 | CTGCAATGAT GTGGCAAA |
| 108 | 4468-4485 | TCTGCAATGA TGTGGCAA |
| 109 | 4469-4486 | TTCTGCAATG ATGTGGCA |
| 110 | 4470-4487 | CTTCTGCAAT GATGTGGC |
| 111 | 5062-5079 | GCTGCCCACT TGCATACT |
| 112 | 5569-5586 | GTTGGCAGAA CATAGAAC |
| 113 | 5570-5587 | CGTTGGCAGA ACATAGAA |
| 114 | 5571-5588 | GCGTTGGCAG AACATAGA |
| 115 | 5616-5633 | ATGGGGCTAC AGGGGATA |
| 116 | 5617-5634 | TATGGGGCTA CAGGGGAT |
| 117 | 5618-5635 | TTATGGGGCT ACAGGGGA |
| 118 | 5620-5637 | AGTTATGGGG CTACAGGG |
| 119 | 5621-5638 | AAGTTATGGG GCTACAGG |
| 120 | 5622-5639 | CAAGTTATGG GGCTACAG |
| 121 | 5623-5640 | CCAAGTTATG GGGCTACA |
| 122 | 5624-5641 | TCCAAGTTAT GGGGCTAC |
| 123 | 5625-5642 | ATCCAAGTTA TGGGGCTA |
| 124 | 5626-5643 | TATCCAAGTT ATGGGGCT |
| 125 | 5627-5644 | CTATCCAAGT TATGGGGC |
| 126 | 5759-5776 | ATTGGAGGAA ATAGGGTG |
| 127 | 5783-5800 | GTCTTGTAGG TAGCAGCC |
| 128 | 5784-5801 | GGTCTTGTAG GTAGCAGC |
| 129 | 5785-5802 | TGGTCTTGTA GGTAGCAG |
| 130 | 5786-5803 | CTGGTCTTGT AGGTAGCA |
| 131 | 5787-5804 | TCTGGTCTTG TAGGTAGC |
| 132 | 5788-5805 | GTCTGGTCTT GTAGGTAG |
| 133 | 5789-5806 | AGTCTGGTCT TGTAGGTA |
| 134 | 5790-5807 | GAGTCTGGTC TTGTAGGT |
| 135 | 5791-5808 | GGAGTCTGGT CTTGTAGG |
| 136 | 5792-5809 | AGGAGTCTGG TCTTGTAG |

The sequences of Table 2 can be chemically-modified to provide active variants thereof, LNA variants thereof, as well as gapmer variants thereof, as known in the art. The sequences of Table 2 can be used in any combination as active agents, such as pooling combinations.

Examples of agents of this disclosure for inhibiting or suppressing expression of TGF-β3 include artemisinin extracts, a pharmaceutically-acceptable salt, salt polymorph, ester, or isomer thereof, and any combination thereof. In some embodiments, this disclosure includes a substantially pure artemisinin having a purity of at least 60%, or 70%, or 80%, or 90%, or 95%.

In certain embodiments, agents of this disclosure for inhibiting or suppressing expression of TGF-β3 may be prepared from a lyophilized powder of the agent.

In some embodiments, a TGF-β2-specific antisense oligonucleotide of this invention may have no more than one or two mismatches as compared to a target human TGF-β2.

In certain embodiments, a TGF-β2-specific antisense oligonucleotide of this invention may reduce a TGF-β2 transcript level by at least 60%, or at least 70%, or at least 80%, or at least 90%.

In additional embodiments, a TGF-β2-specific antisense oligonucleotide of this invention may be selective for TGF-β2 and reduce any TGF-β1 transcript level and any TGF-β3 transcript level by less than 10%, or less than 5%, or less than 1%.

In further embodiments, a therapeutically effective amount of an antisense agent for inhibiting or suppressing expression of TGF-β2 can be from 0.1 to 3000 mg per day, or 1 to 1000 mg per day, or 2 to 500 mg per day, or 2 to 200 mg per day.

In certain embodiments, a formulation of an antisense agent for inhibiting or suppressing expression of TGF-β2 can have a concentration of from 0.05 to 50 μM, or 0.1 to 25 μM, or 0.1 to 10 μM, or 0.1 to 7.5 μM, or 0.1 to 5 μM.

In certain embodiments, a method for using an antisense agent for inhibiting or suppressing expression of TGF-β2 can use a dosage of from 1 to 1000 mg/m²/day, or from 1 to 500 mg/m²/day, or from 1 to 250 mg/m²/day, or from 1 to 100 mg/m²/day, or from 1 to 50 mg/m²/day. Mean human body surface area can be about 1.6 to 1.9 m².

In additional embodiments, a method for using an antisense agent for inhibiting or suppressing expression of TGF-β2 can use a dosage of from 0.05 to 40 mg/kg/day, or from 0.1 to 30 mg/kg/day, or from 0.2 to 20 mg/m²/day, or from 0.3 to 10 mg/m²/day, or from 0.5 to 5 mg/m²/day. Mean human body weight can be about 60 kg.

In some examples and embodiments, an agent may be a TGF-β2-specific antisense oligonucleotide selected from SEQ ID NOs:1-136, and administered or used by continuous intraventricular or intrathecal or intracerebral administration at a dose of 4 μl/min at a dose level of 10 μM on Days 1 to 7, or at a dose of 20 μM on Days 1 to 7, or at a dose of 40 μM on Days 1 to 7, or at a dose of 80 μM on Days 1 to 7.

In some examples and embodiments, an agent may be a TGF-β2-specific antisense oligonucleotide selected from SEQ ID NOs:1-136, and administered or used by continuous intraventricular or intrathecal or intracerebral administration at a dose of 4 μl/min, or 2-8 μl/min, at a dose level of 2 μM on Days 1 to 7, or at a dose of 4 μM on Days 1 to 7, or at a dose of 8 μM on Days 1 to 7, or at a dose of 10 μM on Days 1 to 7.

In some embodiments, an agent may be a TGF-β2-specific antisense oligonucleotide selected from SEQ ID NOs:1-136, and chemically-modified variants thereof, and administered as bolus injection into the ommaya-like reservoir at concentrations of 61.43 mg/ml (10 μM), 1 mg/ml, 7.35 mg/ml, 15 mg/ml, or 18.23 mg/ml.

In further embodiments, an agent may be a TGF-β2-specific antisense oligonucleotide selected from SEQ ID NOs:1-136 and chemically-modified variants thereof, and administered or used by continuous infusion, either singly or in combination with artemisinin in any form at a dose of 500 mg per day taken orally on Days 1 to 5.

Embodiments of this invention contemplate methods and uses comprising an agent which may be a TGF-β2-specific antisense oligonucleotide selected from SEQ ID NOs:9-136.

Examples of agents of this disclosure for inhibiting TGF-β include agents for specifically inhibiting TGF-β1, TGF-β2, or TGF-β3, preferably TGF-β2.

Embodiments of this invention involving administration or use of a composition of an agent can ameliorate or suppress symptoms due to TGF-β induced proteins.

The agent for inhibiting or suppressing expression of TGF-β may be an artemisinin formulation, comprising 90-95% pure artemisinin extract, or a pharmaceutically-acceptable salt, salt polymorph, ester, or isomer thereof, and one or more pharmaceutically acceptable excipients. Excipients may comprise any one or more pharmaceutically acceptable excipients selected from diluents, stabilizers, disintegrants and anticaking agents. In some embodiments, the excipients may comprise any one or more of microcrystalline cellulose, polysorbate 80, crospovidone, croscarmellose sodium, and magnesium stearate.

In further embodiments, the agent for inhibiting or suppressing expression of TGF-β can be an artemisinin compound or derivative thereof, or a pharmaceutically-acceptable salt, salt polymorph, ester, or isomer thereof.

As used herein, a derivative encompasses chemical modifications that provide structural analogs of a compound. For example, substituents or substitutions of an alkyl group can provide structural analogs.

Embodiments of this invention include processes or uses wherein the agent for inhibiting or suppressing expression of TGF-β is a compound, or ligand comprising a small molecule or polypeptide, that interacts with Site I of TGF-β comprising Trp30 and/or Site II of TGF-β comprising Arg15, Gin19, and Phe8, or a pharmaceutically-acceptable salt, salt polymorph, ester, or isomer thereof.

In some embodiments, the agent for inhibiting or suppressing expression of TGF-β may be a polypeptide or peptide mimetic of Site I of TGF-β comprising residues Phe24-Lys37 and/or Site II of TGF-β comprising residues Cys7-Gln19, or a pharmaceutically-acceptable salt, salt polymorph, ester, or isomer thereof.

In further embodiments, the agent for inhibiting or suppressing expression of TGF-β may be an antibody or antibody fragment, humanized or non-humanized, with affinity for Site I of TGF-β comprising residues Phe24-Lys37 and/or Site II of TGF-β comprising residues Cys7-Gln19.

In certain embodiments, the agent for inhibiting or suppressing expression of TGF-β may be a compound comprising three isoprenyl groups and one lactone ring, or derivative thereof, or a pharmaceutically-acceptable salt, salt polymorph, ester, or isomer thereof.

Embodiments of this invention further include pharmaceutical compositions for inhibiting or suppressing expression of TGF-β, or for treating or ameliorating the symptoms of CNS disease in a human or animal. The pharmaceutical compositions may contain a TGF-β inhibitor, artemisinin, pharmaceutically acceptable salts forms, esters, polymorphs or stereoisomers thereof, and any combination thereof, as well as a carrier. The TGF-β inhibitor may be selected from TGF-β2-specific antisense oligonucleotides SEQ ID NOs:1-136 and chemically-modified variants thereof. The carrier may be sterile water for injection, saline, isotonic saline, or a combination thereof.

Importantly, a composition of this disclosure may be substantially free of excipients. Compositions of this invention which are substantially free of excipients have been found to be surprisingly stable in a carrier. In some embodiments, the composition may be stable for at least 14 days, or at least 21 days, or at least 28 days in a carrier at 37° C. In further embodiments, there may be less than 10% reduction in antisense active agent concentration after 90 days in-use.

In additional embodiments, a pharmaceutical composition for infusion may contain less than 1% by weight of excipients, or less than 0.5% by weight of excipients, or less than 0.1% by weight of excipients.

Embodiments of this invention further contemplate therapeutic modalities in which a composition of this invention is administered or utilized in combination with a standard of care therapy for the disease. Examples of additional medicaments which may be administered or utilized in combination with a composition of this invention include anti-inflammatories, anti-inflammatory steroids, piperiquine, pyronaridine, curcumin, frankincense, Remdesivir, Sompraz D, Zifi CV/Zac D, CCM, Broclear, Budamate, Rapitus, Montek LC, low molecular weight heparine, prednisolone, Paracetamol, Vitamin B complex, Vitamin C, Pantoprozol, Doxycycline, Ivermectin, Zinc, Foracort Rotacaps inhalation, Injection Ceftriaxone, Tab Paracetamol, Injection Fragmin, Tablet Covifor, Azithromycin, Injection Dexamethasone, Injection Ondansetron, Tablet Multivitamin, Tablet Ascorbic Acid, Tablet Calcium Carbonate, and Tablet Zinc Sulfate.

This invention further provides kits comprising a lyophilized powder in a vial at a content of 250 mg each of one or more TGF-β2-specific antisense oligonucleotides selected from SEQ ID NOs:1-136.

This invention also provides kits comprising a lyophilized powder in a vial at a content of 500 mg of artemisinin or a derivative thereof, or a compound, or ligand comprising a small molecule or polypeptide, that interacts with Site II of TGF-β comprising Arg15, Gln19, and Phe8, a sesquiterprene lactone or derivative thereof, or a compound comprising three isoprenyl groups and one lactone ring and derivatives thereof, or a pharmaceutically-acceptable salt, salt polymorph, ester, or isomer thereof, or any combination of the foregoing.

Some TGF-β agents are given in U.S. Pat. Nos. 9,963,703, 9,758,786, and 8,476,246.

### Infusion Device and Methods

This invention provides a novel device and methods of use for continuous infusion of agents to treat CNS diseases such as CNS cancers through intracranial routes.

Devices and methods of this invention can deliver agents to the brain or spinal regions using a pumped, continuous infusion system.

The device can be operated within a radiation therapy device and/or a field device, and the pharmaceutical composition may comprise an agent described above and any combination thereof.

In some examples and embodiments, a device of this invention may include a portable extracorporeal pump with a fluid reservoir that is connected via an infusion device to an infusion catheter placeable to any tissue or tumor, and preferably intraventricular space. The fluid may be administered by high flow or continuous perfusion. The device of this invention enables infusion of fluids of any kind by continuous infusion, or continuous convection enhanced delivery. The device of this invention may contain and deliver various pharmaceutical compositions, drugs, proteins, protein toxins, antibodies for treatment or imaging, proteins in enzyme replacement therapy, growth factors and viruses or oligonucleotides in gene therapy.

The device of this invention effectively delivers a therapeutically-effective amount of a pharmaceutical composition to a subject that allows continuous infusion of the agent to a specific location within a subject, for example, a particular tissue or tumor, preferably the ventricular space.

In some aspects (FIGS. 1 and 3), the device of this invention may act as a portable, external convection enhanced delivery device to infuse a pharmaceutical agent in a liquid form to a specific location within a subject. Such aspects of the device can provide for perfusion delivery at a hospital or in-patient center.

In further aspects (FIGS. 2 and 4), the device of this invention may act as a portable indwelling infusion device to an implantable ommaya in a tissue or tumor, preferably the ventricular space, of a subject. Such aspects of use can provide for perfusion delivery outside a patient center, such as for home infusion. Such aspects of use can also provide for perfusion delivery outside a patient center, such as for home infusion, using for example an abdominally-implanted pump.

The device of this invention may inject fluid for continuous infusion.

The device of this invention can be used for delivering various therapeutic agents, such as, for example, drugs, proteins, protein toxins, imaging agents, antibodies for treatment or imaging, proteins in enzyme replacement therapy, growth factors, and/or viruses or oligonucleotides in gene therapy.

The device of this invention can advantageously improve bioavailability and safety, as well as improve the pharmacokinetic and pharmacodynamic properties of the delivered therapeutic agent.

The device of this invention may further enable out-patient treatment with infusion delivery using a portable pump.

The device of this invention (FIGS. 2 and 4) may use an access port indwelling system for infusion, which has the advantage of readily changing the reservoir and pump, and also the pharmaceutical composition contained therein.

The access port in the device of this invention (FIGS. 2 and 4) allows for exact placement of the infusion catheter in a one-step surgical procedure into the center of a tissue or tumor, preferably the ventricular space. The access port can advantageously be located to minimize mechanical stress.

The device of this invention has the advantage to provide clinically useful constant flows rates for continuous infusion or convection enhanced delivery.

The device of this invention has the advantage of providing small-step flow characteristics with portable pumps, in flow rates from 0.01 to 3000 ml/hr, or from 0.01 to 100 ml/hr, or from 0.01 to 2 ml/hr, or from 0.01 to 1 ml/hr, or from 0.05 to 0.5 ml/hr.

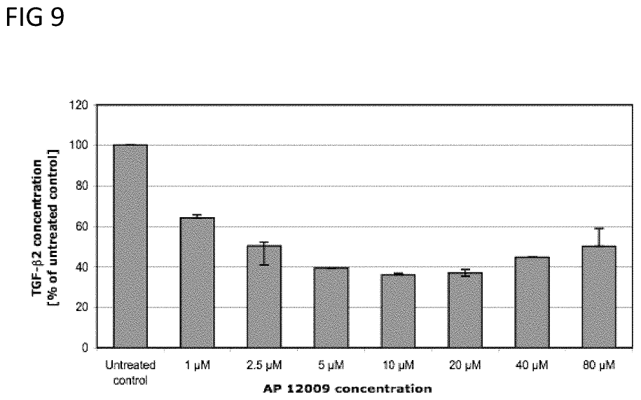

The device of this invention may include an ommaya-like delivery device for use in a method for delivering a pharmaceutical composition by intracranial continuous infusion (FIG. 5). The ommaya-like delivery device shown in FIG. 5 can be used for example as the ommaya reservoir [111] in FIGS. 1-2. The ommaya delivery device includes a non-flexible hard shell [403] composed of an inert material such as metal or hard plastic, which encloses and defines a reservoir for a drug containing fluid. The ommaya delivery device also includes a flexible top [401] composed of a flexible material such as rubber, elastomer, or plastic. The area of the flexible top [401] relative to the entire shell ([401] plus [403]) can be from 10% flexible to 50% flexible. The ommaya delivery device further includes a non-flexible mounting plate [405] composed of an inert material such as metal or hard plastic. The ommaya delivery device further includes port [407] for connecting infusion lines to be in fluid communication with the ommaya delivery device. For example, port [407] can connect to infusion tube [103] in FIGS. 1-2. Fluid communication between the reservoir defined by the shell [403] and the infusion lines is provided by the interior channel [421]. The ommaya delivery device further includes an entry catheter [413] in fluid communication with the ommaya reservoir, wherein the entry catheter is substantially linear and enters intraventricularly into a target region of the brain. Fluid communication between the reservoir defined by the shell [403] and the entry catheter is provided by the interior channel [423]. In another embodiment, hard shell [403] can be a separate protective collar that can be placed over a completely flexible top having the entire area of [401] plus [403] to reduce the exposed area of the flexible top to the area of [401] (see FIG. 8).

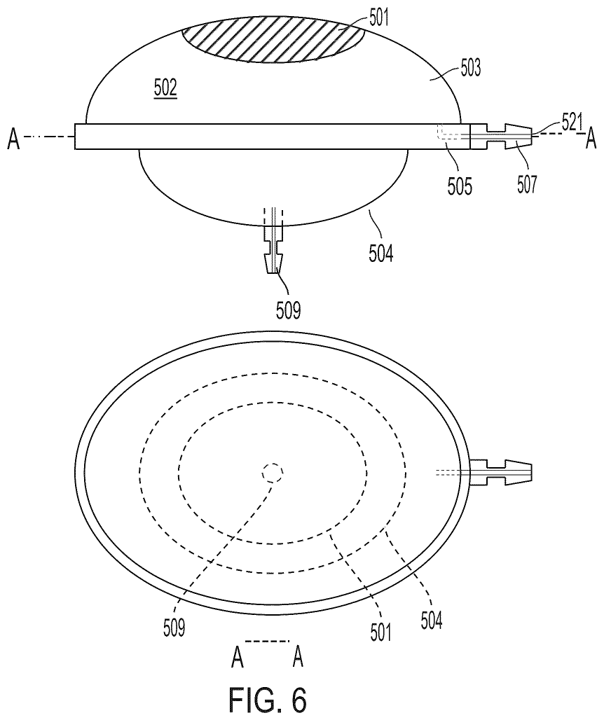

In a further example or embodiment, the device of this invention may include an ommaya-like delivery device for use in a method for delivering a pharmaceutical composition by intracranial continuous infusion (FIG. 6). FIG. 6 shows an ommaya-like delivery device for use in a method for delivering a pharmaceutical composition by intrathecal or intraventricular continuous infusion. The ommaya delivery device shown in FIG. 6 can be used for example as the ommaya reservoir [111] in FIGS. 1-2. The ommaya delivery device includes a non-flexible hard shell [503] and [504] composed of an inert material such as metal or hard plastic. The ommaya delivery device also includes a flexible top [501] composed of a flexible material such as rubber, elastomer, or plastic. The area of the flexible top [501] relative to the entire shell ([501] plus [503]) can be from 10% flexible to 50% flexible, as shown in the lower view of FIG. 6 (view A-A). The ommaya delivery device further includes a non-flexible mounting plate [505] composed of an inert material such as metal or hard plastic. The ommaya delivery device further includes ports [507] and [509] for connecting infusion lines to be in fluid communication with the ommaya delivery device. For example, port [507] can connect to indwelling tube [109] in FIGS. 3-4. Fluid communication between the reservoir defined by the shell [503] and the infusion lines is provided by the interior channel [521]. For example, port [509] can connect to entry catheter [113] in FIGS. 1-2. In another embodiment, hard shell [503] can be a separate protective collar that can be placed over a completely flexible top having the entire area of [501] plus [503] to reduce the exposed area of the flexible top to the area of [501] (see FIG. 8).

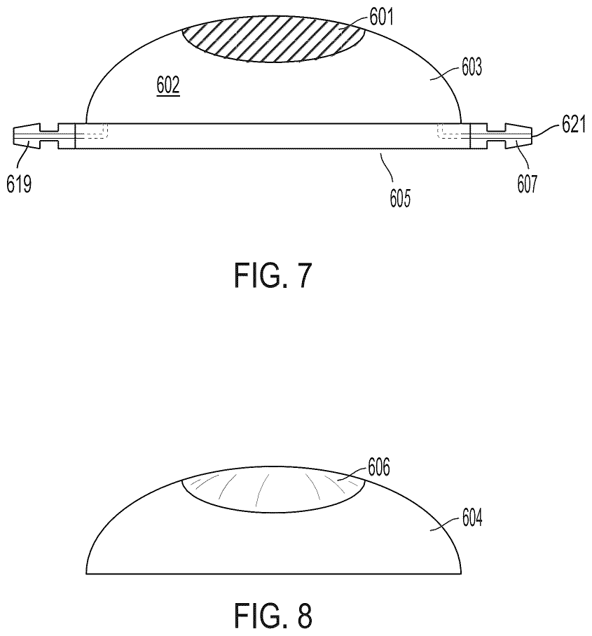

In a further example or embodiment, the device of this invention may include an ommaya-like delivery device for use in a method for delivering a pharmaceutical composition by intracranial continuous infusion (FIG. 7). FIG. 7 shows an ommaya-like delivery device for use in a method for delivering a pharmaceutical composition by intrathecal or intraventricular continuous infusion. The ommaya delivery device shown in FIG. 7 can be used for example as the ommaya reservoir [111] in FIGS. 3-4. The ommaya delivery device includes a non-flexible hard shell [603] composed of an inert material such as metal or hard plastic. The ommaya delivery device also includes a flexible top [601] composed of a flexible material such as rubber, elastomer, or plastic. The ommaya delivery device further includes a non-flexible mounting plate composed of an inert material such as metal or hard plastic. The ommaya delivery device further includes ports [607] and [619] for connecting infusion lines to be in fluid communication with the ommaya delivery device. For example, port [607] can connect to infusion tube [103] in FIGS. 3-4. Fluid communication between the reservoir defined by the shell [603] and the infusion lines is provided by the interior channel [621]. For example, port [619] can connect to entry catheter [113] in FIGS. 3-4. Fluid communication between the reservoir defined by the shell [603] and the entry catheter is provided by the interior channel [619]. In another embodiment, hard shell [603] can be a separate protective collar that can be placed over a completely flexible top having the entire area of [601] plus [603] to reduce the exposed area of the flexible top to the area of [601] (see FIG. 8).

The device of this invention can deliver pharmaceutical agents including any agent suitable for delivery in a solvent system, or, for example, formulated for delivery in an aqueous solution. Such pharmaceutical agents include, for example, analgesics, agents for the treatment of wounds, analeptics, anesthetics, anthelmintics, anticoagulatives, antirheumatics, antiallergics, antiarrhythmics, antibiotics, antidementias, antidiabetics, antidotes, antiepileptics, antihemorrhagics, antihypertonics, anti-migraine preparations, antimycotics, antineoplastics, anti-Parkinson agents, antiphlogistics, antisense oligonucleotides, antituberculosis drugs, anti-arterio-sclerotic agents, biologic materials, blood flow stimulants, corticoids, cytokines, cytostatics, diagnostics, fibrinolytics, geriatrics, gonadotropins, hepatics, hormones and their inhibitors, hypnotics, immunoglobulins, immuno-modulators, immunotherapeutics, organ perfusion solvents, proteins, protein toxins, protectives, sedatives, cardiac drugs, depressants and stimulants, minerals, muscle relaxants, neurotropic agents, oligonucleotides, ophthalmics, vaccines, spasmolytics, urologics, drugs, proteins, protein toxins, antibodies for treatment, proteins in enzyme replacement therapy, growth factors, vectors, viruses in gene therapy and/or agents for diagnosis as agents or antibodies for imaging, x-ray contrast mediums, oligonucleotides inhibiting expression.

The device of this invention can deliver pharmaceutical agents or active substances dissolved or suspended in a physiological solvent or in any other appropriate solvent. The agents may in the form of a free base, or a salt, hydrate, ester, amide, enantiomer, isomer, tautomer, polymorph, prodrug, or derivative of these compounds. The above mentioned agents as well as combinations thereof can be used in the apparatuses, methods, kits, combinations, and compositions herein described.

The device of this invention can have internal surfaces coated with the therapeutic agent.

Components of the device of this invention can be fabricated from a range of materials including, for example, a metallic, a polymeric, and/or a composite material, including materials such as titanium, high grade steel, aluminum, alloys, polymeric foams, plastics, stainless steel and/or metal, and combinations, mixtures, and modification thereof. Materials intended for implantation into a subject can be made of biocompatible materials, such as, for example, polymers, polymeric segments of polystyrene, polyolefins, polyamides, or polyurethane, and metals. Selection of such materials depends on a number of factors including the mechanical properties desired, and the porosity, surface properties, or toxicity of the material. Components of the device of this invention can be made of titanium, an alloy, stainless steel, a ceramic, silicon, Teflon, polypropylene, polyethylene, polystyrene, polyolefins, polyimide, polyamides, polyurethane, PET, PETG, PE, PIG, HDPE, PC, PVC, nylon, urethane, and/or a co-polymer, for example, and may be laminated with or otherwise include a layer of gold, silver and/or aluminum to minimize permeability to gas and liquids, sputtered or otherwise deposited or incorporated therein. Bio-Span® segmented polyurethane-urea, Bionate® polycarbonate urethane, Elasthane™, and Elasthane™ polyetherurethane, which can be used in chronically-implanted medical devices. Elasthane™ has a chemical structure and properties similar to Pellethane® 2363. Thermoplastic silicone-urethane co-polymers such as PurSil™ silicone polyetherurethane and CarboSil™ silicone polycarbonate urethane can also be used in the present device.

The device of this invention may include a filter at any appropriate location. The filter may comprises a sterile filter for removing pathogens, a biological filer for biological materials such as proteins and/or antibodies; a particle filter for removing particulates; a chemical filter for removing chemicals; and/or a filter for removing air or gasses from the solvent. In one aspect, each filter may have a distal port and a proximal port and a lumen therethrough. Within the lumen of each filter there may be a membrane or any other appropriate device for removing air, particles, chemicals, and/or biological materials, such as, for example, pathogens including bacteria, fungi, and/or viruses. The filter can have separate casings, or have common casings. In one aspect, the filter for removing air may be placed extra corporal. In another aspect, the filter for removing particles can be positioned upstream from the sterile filter. The sterile filter typically has by a pore diameter of about 0.45μ or less, or about 0.22μ or less, or about 0.1μ or less. The particle filter may have a pore diameter of greater than about 0.45μ, or greater than about 0.22μ.

The access port can be subcutaneously implanted, for example, over a rib. The access port chamber is in fluid communication with the ommaya reservoir.

The device of this invention can contain compounds to be infused to the subject formulated as an injectable formulation, for example, an aqueous solution or suspension of the compounds suitable for intravenous delivery. When preparing the composition for injection, particularly for intravenous delivery, illustratively, the continuous phase comprises an aqueous solution of tonicity modifiers, buffered to a pH below 7, for example, or below 6, for example. The tonicity modifiers comprise, for example, sodium chloride, glucose, mannitol, trehalose, glycerol, or other pharmaceutical agents that renders the osmotic pressure of the formulation isotonic with blood.

The device of this invention can contain a preservative added to the formulation. A preservative includes benzalkonium chloride, propylparaben, butylparaben. chlorobutanol, belizyl alcohol, phenol. sodium benzoate, or EDTA.

The device of this invention can contain a pharmaceutically acceptable carrier. The carrier materials that can be employed in making the compositions of the present invention are any of those commonly used excipients in pharmaceutics and should be selected on the basis of compatibility with the pharmaceutical agent and the release pro-file properties of the desired dosage form.

The device of this invention can contain excipients such as are known in Remington's Pharmaceutical Sciences, Mack Publishing Co., Easton, Pa. 1975, and Liberman, H. A. and Lachman, L., Eds., Pharmaceutical Dosage Forms, Marcel Decker, New York, N.Y., 1980.

As used herein, the term agent can refer to one or more active compounds, a combination of active compounds, or a composition containing one or more active compounds and a carrier, and/or a solvent, and/or any number of excipients. In some embodiments, the composition may be a pharmaceutical composition. In certain embodiments, the composition may be a pharmaceutical composition containing a therapeutically effective amount of one or more active compounds. Some examples of excipients are given in Liberman, H. A. and Lachman, L., Eds., Pharmaceutical Dosage Forms, Marcel Decker, New York, N.Y., 1980.

Methods for determining a therapeutically effective amount of a compound are known in the art. A therapeutically effective amount can also be determined with routine experimentation, for example, by monitoring a subject's response to administration of an agent and adjusting the dosage. See for example, Remington, The Science and Practice of Pharmacy (Gennaro ed. 20th edition) (2000).

### Operation of the Device

In operation, the device can be adjusted to the patient by performing a computed tomography scan (CT), a brain MRI, or other suitable imaging method on the patient to determine the position of the ventricular space and the length of the catheter needed. For placement of the device, a burr hole is generated and the brain entry catheter is inserted slowly until fluid comes back out of the catheter. The ventricular catheter ommaya-like device is then connected to the external or indwelling infusion components.

For example, an approved silicone micro-catheter is placed stereotactically into the ventricular space. The target position of the entry catheter tip can be pre-calculated from the CT scan or brain MRI. No concurrent imaging is needed for intraventricular placement of the entry catheter. The external or indwelling infusion components can be prefilled with normal saline. Postoperative position of the ommaya-like device and device of this invention can be determined by native X-ray, computed tomography scan, brain MRI, or other suitable imaging method. Localization of the catheter tip can be determined in the same manner. In some examples and embodiments, the infusion ports and catheters can be barium-impregnated for imaging purposes.

The infusion lines can be connected to the infusion device under aseptic conditions. For example, an infusion tube plus bacterial micropore filter (up to 0.2 μm) can be used.

In some examples and embodiments, a special port punction needle can be used, for example, GRIPPER PORT-A-CATH™ needle. The therapeutic solution can be passed through the external or indwelling infusion components until the tip of the port punction needle is reached. Air bubbles can be removed from the method.

For treatments, the therapeutic solution, for example antisense oligonucleotide OT-101 can be infused into the ventricular space. The therapeutic solution can be prepared and used to fill the ommaya-like reservoir. At a patient's bedside, an automatic pump is connected to the device. Before start of the infusion, the infusion device can be filled with therapeutic solution and connected to the port by inserting the port punction needle through its membrane under aseptic conditions.

In some examples and embodiments, a therapeutic solution of OT-101 can be infused continuously at a flow rate of 4 μl/min, or 2-8 μl/min.

In some examples and embodiments, a therapeutically effective amount of an antisense (OT-101 etc.) formulation can be infused continuously at a flow rate of 0.5-20 μl/min, or 1-20 μl/min, or 2-10 μl/min, or 2-8 μl/min, or 1 μl/min, or 2 μl/min, or 3 μl/min, or 4 μl/min, or 5 μl/min, or 6 μl/min, or 7 μl/min, or 8 μl/min, and the agent concentration can be 1-100 μM, or 1-80 μM, or 1-50 μM, or 1-20 μM, or 1-10 μM, or 1 μM, or 2 μM, or 3 μM, or 4 μM, or 5 μM, or 6 μM. Such administrations can be made in a subject on Days 1-3, or Days 1-7, or Days 1-14, or Days 1-21, or Days 1-50 of a regimen or cycle.

### Methods, Compositions and Combinations for CNS Disease

The device of this invention can be used for treating CNS disease such as CNS cancer or ameliorating the symptoms of CNS disease in a human subject or animal in need. The device of this invention may contain a pharmaceutical composition for continuous infusion to the brain or spine.

A pharmaceutical composition for continuous infusion may include an agent for inhibiting or suppressing expression of TGF-β, and administering a therapeutically effective amount of the composition to the subject. This invention provides therapies for treating CNS disease such as cancer or ameliorating symptoms of the CNS disease.

Examples of anti-TGF-β2 agents include TGF-β inhibitors such as antisense oligonucleotides, artemisinin, pharmaceutically acceptable salts forms, esters, polymorphs or stereoisomers thereof, as well as combinations thereof.

In some aspects, this invention provides a kit and device containing a composition of an agent for inhibiting or suppressing expression of TGF-β for treating or ameliorating the symptoms of CNS disease in a human subject or animal.

In further aspects, this invention provides uses of a composition of an agent for inhibiting or suppressing expression of TGF-β in the preparation of a medicament for treating or ameliorating the symptoms of CNS disease in a human subject or animal.

As used herein, “intraventricular” infusion may be used for continuous infusion of a pharmaceutical composition to treat CNS disease including cancer. An example of intraventricular administration is an ommaya-like device with entry catheter.

As used herein, the term intracranial encompasses intrathecal and intraventricular. For example, intracranial infusion includes intrathecal infusion and intraventricular infusion. Further, the term “intrathecal and intraventricular” is intended to encompass “intracranial.”

In certain embodiments, an agent, use or method of this invention, upon administration to a subject can reduce the level of TGF-β2 in the subject, which can be referred to as an improved TGF-β2 signature.

In some aspects, an agent, use or method of this invention, upon administration or use by subjects, may decrease mortality rate at month 6, 12, 18, 24, 30, or 36.

In further aspects, an agent, use or method of this invention, upon administration or use by subjects, may increase survival rate at month 6, 12, 18, 24, 30, or 36. Survival can be determined as overall survival or progression free survival.

In some aspects, an agent, medicament or administration that is substantially free of excipients may comprise a carrier.

Embodiments of this invention further contemplate using TGF-β2 as a selective biomarker for providing improved outcome for cancer therapy with agents of this invention in combination with radiation therapy.

This invention provides methods for therapy by selecting patients based on TGF-β2 levels as a biomarker for improved outcome for radiation therapy in cancer.

Surprisingly improved overall survival and survival following radiation therapy was found with patients having low TGF-β2 expression, as compared to patients having high TGF-β2 expression, over a wide range of expression levels. No such differences were observed for TGF-beta-1 and TGF-beta-3. Thus, reduced TGF-β2 can be used as a successful biomarker for selecting patients who will benefit in such therapies.

In further studies, overall survival in pediatric glioma under radiation therapy was surprisingly improved using TGF-β2 as a selector. TGF-beta-1 and TGF-beta-3 were not predictive of survival.

In some embodiments, TGF-β2 level can be used as a surprisingly effective biomarker for selecting patients who will benefit from radiation therapy in cancer in combination with chemotherapy such as temozolomide (TMZ), and/or TMZ plus radiation therapy, and/or antiangiogenic therapy such as bevacizumab. No such predictive results were observed for TGF-β1 and TGF-β3.

In some embodiments, this invention provides agents, uses, and methods which combine inhibiting or suppressing expression of TGF-β2 for treating or ameliorating the symptoms of a CNS disease in a human subject or animal with medicaments that are a targeted cancer drug, a cancer growth blocker, or an EGFR inhibitor.

In certain embodiments, this invention provides agents, uses, and methods which combine inhibiting or suppressing expression of TGF-β2 for treating or ameliorating the symptoms of a CNS disease in a human subject or animal with bevacizumab, everolimus, belzutifan, dabrafenib, trametinib, and combinations thereof.

In additional embodiments, this invention provides agents, uses, and methods which combine inhibiting or suppressing expression of TGF-β2 for treating or ameliorating the symptoms of a CNS disease in a human subject or animal with erlotinib, gefitinib, afatinib, osimertinib, dacomitininb, and combinations thereof.

In further embodiments, this invention provides agents, uses, and methods which combine inhibiting or suppressing expression of TGF-β2 for treating or ameliorating the symptoms of a CNS disease in a human subject or animal with temozolomide.

### Human TGF-β2-Specific Phosphorothioate Antisense Oligodeoxynucleotide

An antisense oligonucleotide (ASO) can be a single-stranded deoxyribonucleotide, which may be complementary to an mRNA target. The antisense therapy may downregulate a molecular target, which may be achieved by induction of RNase H endonuclease activity that cleaves the RNA-DNA heteroduplex with a significant reduction of the target gene translation. Other ASO mechanisms can include inhibition of 5′ cap formation, alteration of splicing process such as splice-switching, and steric hindrance of ribosomal activity.

Antisense therapeutic strategies can utilize single-stranded DNA oligonucleotides that inhibit protein production by mediating the catalytic degradation of a target mRNA, or by binding to sites on mRNA needed for translation. Antisense oligonucleotides can be designed to target the viral RNA genome or viral transcripts. Antisense oligonucleotides can provide an approach for identifying potential targets, and therefore represent potential therapeutics.

Antisense oligonucleotides can be small synthetic pieces of single-stranded DNA that may be 15-30 nucleotides in length. An ASO may specifically bind to a complementary DNA/RNA sequence by Watson-Crick hybridization and once bound to the target RNA, inhibit the translational processes either by inducing cleavage mechanisms or by inhibiting mRNA maturation. An ASO may selectively inhibit gene expression with specificity. Chemical modifications of DNA or RNA can be used to increase stability.

For example, modifications can be introduced in the phosphodiester bond, the sugar ring, and the backbone. ASO antiviral agents may block translational processes either by (i) ribonuclease H (RNAse H) or RNase P mediated cleavage of mRNA or (ii) by sterically (non-bonding) blocking enzymes that are involved in the target gene translation. Human TGF-β2-specific phosphorothioate antisense oligodeoxynucleotide (OT-101; AP 12009; Trabedersen), hereafter referred to as OT-101 or AP 12009, is intended to reduce the level of TGF-β2 protein in malignant gliomas, and thereby delay the progression of disease.

Antisense oligodeoxynucleotides are short strings of DNA that are designed to downregulate gene expression by interfering with the translation of a specific encoded protein at the mRNA level. OT-101 is a synthetic 18-mer phosphorothioate oligodeoxynucleotide (S-ODN) where all 3′-5′ linkages are modified to phosphorothioates. The molecular formula is C₁₇₇H₂₀₈N₆₀Na₁₇O₉₄P₁₇S₁₇ and the molecular weight 6,143 g/mol. OT-101 was designed to be complementary to a specific sequence of human TGF-β2 mRNA following expression of the gene.

OT-101 is currently supplied as a lyophilized powder in 50-mL glass vials in three different quantities. OT-101 lyophilized powder is dissolved in isotonic (0.9%) aqueous sodium chloride prior to use.

Examples of agents of this disclosure for inhibiting or suppressing expression of TGF-β include antisense oligonucleotides specific for TGF-β1, TGF-β2, or TGF-β3.

Examples of agents of this disclosure for inhibiting or suppressing expression of TGF-β2 include TGF-β2-specific antisense oligonucleotides given in SEQ ID NOs:1-136 in Table 2, including SEQ ID NO:8, cggcatgtct attttgta. (OT-101).

Antisense oligonucleotides given in Table 2 herein can be chemically-modified, as known in the art.

Examples of agents of this disclosure for inhibiting or suppressing expression of TGF-β include artemisinin extracts, a pharmaceutically-acceptable salt, salt polymorph, ester, or isomer thereof, and any combination thereof. In some embodiments, this disclosure includes a substantially pure artemisinin having a purity of at least 60%, or 70%, or 80%, or 90%, or 95%.

In certain embodiments, agents of this disclosure for inhibiting or suppressing expression of TGF-β may be prepared from a lyophilized powder of the agent.

In some examples and embodiments, an agent may be a TGF-β2-specific antisense oligonucleotide selected from SEQ ID NOs:1-136, and administered or used by continuous intraventricular or intrathecal or intracerebral administration at a dose of 4 μl/min at a dose level of 10 μM on Days 1 to 7, or at a dose of 20 μM on Days 1 to 7, or at a dose of 40 μM on Days 1 to 7, or at a dose of 80 μM on Days 1 to 7.

In some examples and embodiments, an agent may be a TGF-β2-specific antisense oligonucleotide selected from SEQ ID NOs:1-136, and administered or used by continuous intraventricular or intrathecal or intracerebral administration at a dose of 4 μl/min, or 2-8 μl/min, at a dose level of 2 μM on Days 1 to 7, or at a dose of 4 μM on Days 1 to 7, or at a dose of 8 μM on Days 1 to 7, or at a dose of 10 μM on Days 1 to 7.

In some embodiments, an agent may be a TGF-β2-specific antisense oligonucleotide selected from SEQ ID NOs:1-136, and chemically-modified variants thereof, and administered as bolus injection into the ommaya-like reservoir at concentrations of 61.43 mg/ml (10 μM), 1 mg/ml, 7.35 mg/ml, 15 mg/ml, or 18.23 mg/ml.

In further embodiments, an agent may be a TGF-β2-specific antisense oligonucleotide selected from SEQ ID NOs:1-136 and chemically-modified variants thereof, and administered or used by continuous infusion, either singly or in combination with artemisinin in any form at a dose of 500 mg per day taken orally on Days 1 to 5.

Examples of agents of this disclosure for inhibiting TGF-β include agents for specifically inhibiting TGF-β1, TGF-β2, or TGF-β3, preferably TGF-β2.

Embodiments of this invention involving administration or use of a composition of an agent can ameliorate or suppress symptoms due to TGF-β2 induced proteins.

The agent for inhibiting or suppressing expression of TGF-β may be an artemisinin formulation, comprising 90-95% pure artemisinin extract, or a pharmaceutically-acceptable salt, salt polymorph, ester, or isomer thereof, and one or more pharmaceutically acceptable excipients. Excipients may comprise any one or more pharmaceutically acceptable excipients selected from diluents, stabilizers, disintegrants and anticaking agents. In some embodiments, the excipients may comprise any one or more of microcrystalline cellulose, polysorbate 80, crospovidone, croscarmellose sodium, and magnesium stearate.

In further embodiments, the agent for inhibiting or suppressing expression of TGF-β can be an artemisinin compound or derivative thereof, or a pharmaceutically-acceptable salt, salt polymorph, ester, or isomer thereof.

As used herein, a derivative encompasses chemical modifications that provide structural analogs of a compound. For example, substituents or substitutions of an alkyl group can provide structural analogs.

Embodiments of this invention include processes or uses wherein the agent for inhibiting or suppressing expression of TGF-β is a compound, or ligand comprising a small molecule or polypeptide, that interacts with Site I of TGF-β comprising Trp30 and/or Site II of TGF-β comprising Arg15, Gln19, and Phe8, or a pharmaceutically-acceptable salt, salt polymorph, ester, or isomer thereof.

In some embodiments, the agent for inhibiting or suppressing expression of TGF-β may be a polypeptide or peptide mimetic of Site I of TGF-β comprising residues Phe24-Lys37 and/or Site II of TGF-β comprising residues Cys7-Gln19, or a pharmaceutically-acceptable salt, salt polymorph, ester, or isomer thereof.

In further embodiments, the agent for inhibiting or suppressing expression of TGF-β may be an antibody or antibody fragment, humanized or non-humanized, with affinity for Site I of TGF-β comprising residues Phe24-Lys37 and/or Site II of TGF-β comprising residues Cys7-Gln19.

Embodiments of this invention further include pharmaceutical compositions for inhibiting or suppressing expression of TGF-β, or for treating or ameliorating the symptoms of CNS disease in a human or animal. The pharmaceutical compositions may contain a TGF-β inhibitor, artemisinin, pharmaceutically acceptable salts forms, esters, polymorphs or stereoisomers thereof, and any combination thereof, as well as a carrier. The TGF-β inhibitor may be selected from TGF-β2-specific antisense oligonucleotides SEQ ID NOs:1-136 and chemically-modified variants thereof. The carrier may be sterile water for injection, saline, isotonic saline, or a combination thereof.

Importantly, a composition of this disclosure may be substantially free of excipients. Compositions of this invention which are substantially free of excipients have been found to be surprisingly stable in a carrier. In some embodiments, the composition may be stable for at least 14 days, or at least 21 days, or at least 28 days in a carrier at 37° C.

In additional embodiments, a pharmaceutical composition for infusion may contain less than 1% by weight of excipients, or less than 0.5% by weight of excipients, or less than 0.1% by weight of excipients.

Embodiments of this invention further contemplate therapeutic modalities in which a composition of this invention is administered or utilized in combination with a standard of care therapy for the disease. Examples of additional medicaments which may be administered or utilized in combination with a composition of this invention include anti-inflammatories, anti-inflammatory steroids, piperiquine, pyronaridine, curcumin, frankincense, Remdesivir, Sompraz D, Zifi CV/Zac D, CCM, Broclear, Budamate, Rapitus, Montek LC, low molecular weight heparine, prednisolone, Paracetamol, Vitamin B complex, Vitamin C, Pantoprozol, Doxycycline, Ivermectin, Zinc, Foracort Rotacaps inhalation, Injection Ceftriaxone, Tab Paracetamol, Injection Fragmin, Tablet Covifor, Azithromycin, Injection Dexamethasone, Injection Odndansetron, Tablet Multivitamin, Tablet Ascorbic Acid, Tablet Calcium Carbonate, and Tablet Zinc Sulfate.

This invention further provides kits comprising a lyophilized powder in a vial at a content of 3.75 mg each of one or more TGF-β2-specific antisense oligonucleotides selected from SEQ ID NOs:1-136.

This invention also provides kits comprising a lyophilized powder in a vial at a content of 500 mg of artemisinin or a derivative thereof, or a compound, or ligand comprising a small molecule or polypeptide, that interacts with Site II of TGF-β comprising Arg15, Gln19, and Phe8, a sesquiterpene lactone or derivative thereof, or a compound comprising three isoprenyl groups and one lactone ring and derivatives thereof, or a pharmaceutically-acceptable salt, salt polymorph, ester, or isomer thereof, or any combination of the foregoing.

### Delivery of Therapeutics by Infusion

### Delivery of OT-101-TGF-β Antisense—for the Treatment of Glioblastoma.

OT-101 is a TGF-β2 antisense and was active against recurrent glioblastoma in G004, a phase 2 clinical trial (Uckun FM, Qazi S, Hwang L, Trieu VN. Recurrent or refractory high grade gliomas treated by convection enhanced delivery of a TGF-β2 targeting RNA therapeutic: a post-hoc analysis with long-term follow up. Cancers. 2019, 11:1892).

OT-101 was delivered intratumorally through cerebrally implanted catheter. To further expand the application of OT-101, intrathecal delivery of tritiated OT-101 into Sprague-Dawley CD (albino) rats was explored. Throughout the studies, there were no sex differences.

Surprisingly, after 1 hr intracerebral infusion or intraventricular infusion in rats, OT-101 was similarly widespread in cerebellum, remaining cerebrum, and cerebrospinal fluid (CSF), showing that intracerebral administration was similar to intraventricular administration and accessed the entire CNS compartments through CSF.

OT-101 concentration was stable for the first 4 hours post infusion and decayed biexponentially with a slow terminal half life for tissue but not for CSF, suggestive of rapid penetration of the underlying tissue away from the CSF compartment. Minimum amount of OT-101 was detected in the plasma compartment. Intrathecal bolus administration of 0.1 mL of OT-101 at 14, 30, 200, 300, and 500 uM into cynomolgus monkeys did not result in any single dose toxicity. Histopathology examination revealed no substance-related histomorphological lesions in the cavum subarachnoideale of the lumbar region. No changes were noted in the grey and white matter of the spinal cord, the nerve trunk, and nerve cells did not show any abnormalities. These data suggest that intrathecal administration of OT-101 is a potentially effective delivery route for antisense therapeutics such as OT-101 to the midline i.e. for Diffuse Midline Glioma (DMG).

Targeting Transforming Growth Factor Beta 2 (TGF-β2) with OT-101 for Post-Radiation Consolidation in Diffuse Intrinsic Pontine Glioma

Diffuse intrinsic pontine glioma (DIPG) in children has a dismal prognosis with a median overall survival (OS) of 10 months and a 2-year overall survival rate of <10% after standard radiation therapy.

Chemotherapy does offer clinically meaningful benefits. Therefore, there is an urgent need for therapeutic innovations for treatment of pediatric DIPG. High-grade glioma cells, including pediatric glioblastoma and DIPG cells have been shown to produce transforming growth factor beta 2 (TGF-β2).

(TGFB2) which has been implicated both as promoter of glioma cells and as a key contributor to the T-cell hyporesponsiveness of the tumor microenvironment (TME) towards glioma cells. OT-101 is a first-in-class RNA therapeutic designed to abrogate the immunosuppressive and tumor promoting actions of TGF-B2. At low micromolar concentrations, OT-101 reduces the TGFB2 secretion by human glioma cells, blocks their proliferation as well as migration, and restores the anti-glioma cytolytic function of patient-derived T-cells. Intracerebrally applied OT-101 was administered and showed promising single agent activity in Recurrent/Refractory (R/R) high-grade glioma (HGG) (Uckun et al., Cancers. 2019 28; 11(12):1892). The intrathecal administration of antineoplastic drugs directly in the CSF allows to bypass the selective filter of blood-brain barrier (BBB), achieving significant concentrations of the antineoplastic agents in CSF, while reducing the likelihood of systemic toxicity. Informed by favorable safety pharmacology studies of intrathecally delivered OT-101 in rabbits and primates and encouraged by its single agent activity in adult patients with HGG, pediatric patients with DIPG are treated with OT-101. Multiple doses of OT-101 are administered after completion of radiation therapy as intrathecal bolus injections. The study is designed to determine: 1) the maximum tolerated dose (MTD) or recommended Phase 2 dose (RP2D) of OT-101, and 2) its efficacy in children with DIPG.

Numbered embodiments of this invention may include:
1) An agent for inhibiting or suppressing expression of TGF-β2 for treating or ameliorating the symptoms of a CNS disease in a human subject or animal.
2) Use of an agent for inhibiting or suppressing expression of TGF-β2 in the preparation of a medicament for treating or ameliorating the symptoms of a CNS disease in a human subject or animal.
3) A method for treating or ameliorating the symptoms of a CNS disease in a human subject or animal in need, the method comprising: preparing a composition comprising an agent for inhibiting or suppressing expression of TGF-β2 in a carrier; and administering a therapeutically effective amount of the composition to the subject.
4) The agent, use or method of any of embodiments 1-3, wherein the CNS disease is glioma, glioblastoma, diffuse intrinsic pontine glioma (DIPG), diffuse midline glioma (DMG), leptomeningeal or brain metastasis, brain or spinal cancer, or CNS tumors.
5) The agent, use or method of any of embodiments 1-4, in combination with a medicament comprising a targeted cancer drug, a cancer growth blocker, an EGFR inhibitor, or a combination thereof.
6) The agent, use or method of any of embodiments 1-5, in combination with a medicament selected from bevacizumab, everolimus, belzutifan, dabrafenib, trametinib, and combinations thereof.
7) The agent, use or method of any of embodiments 1-6, in combination with a medicament which is a cancer growth blocker selected from an angiogenesis inhibitor, a histone deacetylase inhibitor, a hedgehog blocker, an mTOR inhibitor, a p53 inhibitor, a PARP inhibitor, a proteasome inhibitor, a tyrosine kinase inhibitor, and combinations thereof.
8) The agent, use or method of any of embodiments 1-7, in combination with a medicament which is an EGFR inhibitor selected from erlotinib, gefitinib, afatinib, osimertinib, dacomitininb, and combinations thereof.
9) The agent, use or method of any of embodiments 1-8, in combination with temozolomide.
10) The agent, use or method of any of embodiments 1-9, in combination with treatment of the CNS disease by radiation therapy or electric field therapy.
11) The agent, use or method of any of embodiments 1-10, wherein the administration or use of the composition or agent is combined with a standard of care treatment for the CNS disease.
12) The agent, use or method of any of embodiments 1-11, wherein the agents, medicaments, therapies, treatments, and administrations are each administered concurrently, simultaneously, sequentially, or separately in time.
13) The agent, use or method of any of embodiments 1-12, wherein each agent and medicament is administered separately or in combination by infusion or injection.
14) The agent, use or method of any of embodiments 1-13, comprising administration or use by intracranial continuous infusion or bolus administration.
15) The agent, use or method of any of embodiments 1-14, wherein the intracranial continuous infusion comprises infusion with an ommaya-like reservoir having a partially-flexible top.
16) The agent, use or method of any of embodiments 1-15, wherein the intracranial continuous infusion comprises a single entry catheter placed into a target region of brain.
17) The agent, use or method of any of any of embodiments 1-16, wherein the subject upon the administration or use has an improved TGF-β2 signature.
18) The agent, use or method of any of embodiments 1-17, wherein the administration or use decreases mortality rate at month 6, 12, 18, 24, 30, or 36.
19) The agent, use or method of any of embodiments 1-18, wherein the administration or use increases survival rate at month 6, 12, 18, 24, 30, or 36.
20) The agent, use or method of any of embodiments 1-19, wherein the agent for inhibiting or suppressing expression of TGF-β2 is selected from TGF-β2-specific antisense oligonucleotides complementary to a TGF-β2 transcript as follows: Table 2 SEQ ID NOs:1-136 and chemically-modified variants thereof, LNA variants thereof, gapmer variants thereof, and any combination thereof.
21) The agent, use or method of any of embodiments 1-20, wherein the agent for inhibiting or suppressing expression of TGF-β2 is a TGF-β2-specific antisense oligonucleotide having no more than one or two mismatches as compared to a target human TGF-β2.
22) The agent, use or method of any of embodiments 1-21, wherein the agent for inhibiting or suppressing expression of TGF-β2 is a TGF-β2-specific antisense oligonucleotide which reduces a TGF-β2 transcript level by at least 60%, or at least 70%, or at least 80%, or at least 90%.
23) The agent, use or method of any of embodiments 1-22, wherein the agent for inhibiting or suppressing expression of TGF-β2 is a TGF-β2-specific antisense oligonucleotide which reduces any TGF-β1 transcript level and any TGF-β3 transcript level by less than 10%, or less than 5%, or less than 1%.
24) The agent, use or method of any of embodiments 1-23, comprising a TGF-β2-specific antisense oligonucleotide having one or more nucleotides chemically modified as a phosphorothioate internucleoside linkage, a methoxypropylphosphonate internucleoside linkage, an aminophosphoro linkage to a morpholino group, a 2′-OMe ribose group, a 2′-MOE methoxyethyl ribose group, a 2′-4′ constrained methoxyethyl bicyclic ribose group, a 2′-4′ constrained ethyl bicyclic ribose group, an LNA ribose group, a 2′-F ribose group, or a 5-methylcytodine base.
25) The agent, use or method of any of embodiments 1-24, wherein the agent is conjugated to a polyethylene glycol, a lipid, or a triantenarry N-acteyl-galactosamine.
26) The agent, use or method of any of embodiments 1-25, comprising a carrier of sterile water for injection, saline, isotonic saline, phosphate buffered saline, or a combination thereof.
27) The agent, use or method of any of embodiments 1-26, wherein the agent, medicament or administration is substantially free of excipients.
28) The agent, use or method of any of embodiments 1-27, wherein the agent, medicament or administration is stable for at least 14 days in carrier at 37° C. while being pumped in intracranial continuous infusion, or wherein there is less than 10% reduction in antisense active agent concentration after 90 days in-use.
29) The agent, use or method of any of embodiments 1-28, wherein the method comprises administering the composition by intracranial infusion at a rate of 2-8 μl/min and an agent concentration of 1-80 μM on Days 1 to 7, preferably for intracranial continuous infusion.
30) A kit comprising:
   an agent comprising a total of 250 mg of one or more TGF-β2-specific antisense oligonucleotides selected from Table 2 SEQ ID NOs:1-136 and chemically-modified variants thereof, LNA variants thereof, gapmer variants thereof, and any combination thereof; and
   an ommaya-like reservoir having a partially-flexible top.
31) A device for delivering a fluid pharmaceutical composition by intracranial continuous infusion, the device comprising:
   a reservoir [117] containing the pharmaceutical composition;
   a pump [101] to force the pharmaceutical composition through an infusion tube [103] into an ommaya reservoir [111], the pump being in fluid communication with the ommaya reservoir, and wherein the pump is in fluid communication with the reservoir through a reservoir tube [115];
   a filter [105] in line with the infusion tube; and
   an entry catheter [113] in fluid communication with the ommaya reservoir, wherein the entry catheter is substantially linear and enters intraventricularly into a target region of the brain.
32) The device of embodiment 31, wherein the entry catheter [113] is non-linear and has one or more bends to enter intraventricularly into a target region of the brain.
33) A device for delivering a fluid pharmaceutical composition by intracranial continuous infusion, the device comprising:
   a reservoir [117] containing the pharmaceutical composition;
   a pump [101] to force the pharmaceutical composition through an infusion tube [103] into an access port [107], the pump being in fluid communication with an ommaya reservoir [111], wherein the reservoir is in fluid communication with the pump through a reservoir tube [115];
   a filter [105] in line with the infusion tube;
   an indwelling tube [109] in fluid communication with the access port and the ommaya reservoir [111]; and
   an entry catheter [113] in fluid communication with the ommaya reservoir, wherein the entry catheter is substantially linear and enters intraventricularly into a target region of the brain.
34) The device of any of embodiments 31-33, wherein the entry catheter [113] is non-linear and has one or more bends to enter intraventricularly into a target region of the brain.
35) The device of any of embodiments 31-34, wherein the ommaya reservoir [111] comprises a partially-flexible top.
36) The device of any of embodiments 31-35, wherein the device provides continuous infusion of a therapeutically effective amount of the fluid pharmaceutical composition to the target region.
37) The device of any of embodiments 31-36, wherein the distal end of the entry catheter enters the target region of the brain.
38) The device of any of embodiments 31-37, wherein the ommaya reservoir [111] holds the pharmaceutical composition behind a membrane for a sustained period of time for sustained release of the pharmaceutical composition to the entry catheter.
39) The device of any of embodiments 31-38, wherein the distal end tip of the entry catheter that enters the brain is a step-down end, a recessed step end, a multi-port end, a microporous end, or a balloon tipped end.
40) The device of any of embodiments 31-39, wherein the pump [101] is a Pegasus Vario, a PEGA PCA, a CADD Solis VIP, a CADD-Legacy PLUS, a CADD-Legacy PCA, a CADD-Legacy 1, or other pump with similar specifications.
41) The device of any of embodiments 31-40, wherein the infusion rate of the fluid pharmaceutical composition is from 0.01 to 3000 ml/hr, or from 0.01 to 100 ml/hr, or from 0.01 to 2 ml/hr, or from 0.01 to 1 ml/hr, or from 0.05 to 0.5 ml/hr.
42) The device of any of embodiments 31-41, wherein the infusion tube [103] or the indwelling tube [109] is a PEGA Line 100 SF 100 cm with a 0.2 μm sterile filter, or a 200 cm infusion line with a 0.2 μm sterile filter, or a Port-a-Cath (#21-4034-24) with 22 G Needle (#21-2737-24) and extension (#21-7106-24), or other tube with similar specifications.
43) The device of any of embodiments 31-42, wherein the fluid pharmaceutical composition comprises an agent for inhibiting or suppressing expression of TGF-β, which can be used for treating or ameliorating the symptoms of a CNS disease in a human subject or animal.
44) The device of any of embodiments 31-43, wherein the fluid pharmaceutical composition comprises microparticles or nanoparticles of an agent, medicament, or delivery vehicle.
45) The device of any of embodiments 31-44, wherein the fluid pharmaceutical composition is therapeutic for CNS disease or CNS cancer.
46) The device of any of embodiments 31-45, wherein the fluid pharmaceutical composition is therapeutic for glioma, glioblastoma, diffuse intrinsic pontine glioma (DIPG), diffuse midline glioma (DMG), leptomeningeal or brain metastasis, brain or spinal cancer, or CNS tumors.
47) The device of any of embodiments 31-46, wherein the fluid pharmaceutical composition comprises an agent for inhibiting or suppressing expression of TGF-β2 selected from TGF-β2-specific antisense oligonucleotides complementary to a TGF-β2 transcript as follows: Table 2 SEQ ID NOs:1-136 and chemically-modified variants thereof, LNA variants thereof, gapmer variants thereof, and any combination thereof.
48) The device of any of embodiments 31-47, wherein the device is operated in combination with radiation therapy, or electric field therapy.
49) A device for delivering a pharmaceutical composition by intracranial continuous infusion, the device comprising:
   a reservoir [402] containing the pharmaceutical composition, the reservoir comprising a hard shell [403], a flexible top [401], and a non-flexible mounting plate [405];
   a port [407] in fluid communication with the reservoir; and
   an entry catheter [413] in fluid communication with the reservoir, wherein the entry catheter is substantially linear and enters intraventricularly into a target region of the brain.
50) A device for delivering a pharmaceutical composition by intracranial continuous infusion, the device comprising:
   a reservoir [502] containing the pharmaceutical composition, the reservoir comprising an upper hard shell [503], a lower hard shell [504], a flexible top [501], and a non-flexible mounting plate [505];
   a port [507] in fluid communication with the reservoir for attaching an infusion line; and
   a port [509] in fluid communication with the reservoir for attaching an entry catheter.
51) A device for delivering a pharmaceutical composition by intracranial continuous infusion, the device comprising:
   a reservoir [602] containing the pharmaceutical composition, the reservoir comprising an upper hard shell [603], a flexible top [601], and a non-flexible mounting plate [605];
   a port [619] in fluid communication with the reservoir for attaching an infusion line; and
   a port [607] in fluid communication with the reservoir for attaching an entry catheter.
52) A collar for a device for delivering a pharmaceutical composition by intracranial continuous infusion, the collar comprising:
   a hard shell [604] having an opening [606] which exposes a flexible top of the device.
53) A method for administering a pharmaceutical composition by intracranial continuous infusion, comprising:
   mounting a device according to any one of embodiments 31-52 to a patient; and
   pumping the pharmaceutical composition in the device to provide intracranial continuous infusion to the patient.
54) The method of embodiment 53, wherein the device is mounted and the entry catheter is placed into the brain without concurrent head or brain imaging.
55) The method of any of embodiments 53-54, wherein the device is mounted and a single entry catheter is placed into the brain.
56) A kit for continuous intracranial infusion of a pharmaceutical composition into a subject, the kit comprising:
   a reservoir containing the pharmaceutical composition;
   a pump;
   an ommaya reservoir having a partially-flexible top;
   infusion tubes for connecting the reservoir to the pump, and connecting the pump to the ommaya reservoir;
   a filter; and
   an entry catheter.
57) The kit of any of embodiments 30 and 56, wherein the entry catheter is substantially linear for intraventricular entry into a target region of the brain.
58) The kit of any of embodiments 30 and 56-57, wherein the entry catheter is non-linear and has a bend for intraventricular entry into a target region of the brain.
59) The kit of any of embodiments 30 and 56-58, wherein the infusion tubes are external to the subject.
60) The kit of any of embodiments 30 and 56-59, wherein a portion of the infusion tube connecting the pump to the ommaya reservoir is indwelling the subject.

All publications including patents, patent application publications, and non-patent publications referred to in this description, as well as the sequence listing are each expressly incorporated herein by reference in their entirety for all purposes.

Although the foregoing disclosure has been described in detail by way of example for purposes of clarity of understanding, it will be apparent to the artisan that certain changes and modifications are comprehended by the disclosure and may be practiced without undue experimentation within the scope of the appended claims, which are presented by way of illustration not limitation. This invention includes all such additional embodiments, equivalents, and modifications. This invention includes any combinations or mixtures of the features, materials, elements, or limitations of the various illustrative components, examples, and claimed embodiments.

It is emphasized herein according to common practice the features of the drawings have arbitrary scale and are intended to cover similar features that may be arbitrarily expanded or reduced.

### EXAMPLES

### Example 1

Examples of pumps for continuous infusion by intrathecal or intraventricular routes are shown in Table 3.

TABLE 3
Examples of pumps for continuous infusion

| Pumps | Manufacturer | Infusion Rate | | Infusing Tube/Accessories |
|---|---|---|---|---|
| Pegasus Vario | LogoMed | 0.01-15 | ml/hr | PEGA Line 100 SF (100 cm) w/ |
| (Isarna) | Venner | | | 0.2 um sterile filter |
| | | | | 200 cm infusion line w/0.2 um |
| PEGA PCA | Medical | 0.1-100 | ml/h | sterile filter |
| | | | | Port-a-Cath (#21-4034-24), |
| CADD Solis | Smiths | 0.1-500 | ml/hr | 22G Needle (#21-2737-24), |
| VIP | Medical | | | Extension set (#21-7106-24) |
| CADD-Legacy | Smiths | 0.1-125 | ml/hr | |
| PLUS | Medical | | | |
| CADD-Legacy | Smiths | | | |
| PCA | Medical | 0-50 | ml/hr | |
| CADD-Legacy | Smiths | | | |
| 1 | Medical | 1-3000 | ml/hr | |

The infusion tube or the indwelling tube may be a PEGA Line 100 SF 100 cm with a 0.2 um sterile filter, or a 200 cm infusion line with a 0.2 um sterile filter, or a Port-a-Cath (#21-4034-24) with 22 G Needle (#21-2737-24) and extension (#21-7106-24), or other tubes with similar specifications.

In some aspects, the brain entry catheter positioning into the ventricular space can be a nonspecific ventricular catheter. The ventricular catheter may have an inner diameter of from 1.0 to 2.0 mm. For example, the ventricular catheter may have an inner diameter of 1.4 mm and an outer diameter of 2.7 mm. The catheter may be 14 cm in length, or shorter, and can be supplied with 24 inlet holes (for example, 3 rows of 8 holes) at the proximal end. In general, the inner diameter of the catheter may dictate the diffusion of the drug into the ventricular space. The ommaya-like reservoir of this invention may have a surface area of from 300 to 400 mm², and the area of the inner opening of the catheter can be from 0.785 mm² to 3.14 mm². Thus the ratio of the reservoir surface area to the catheter surface area will have a range from 96 to 509. With this ratio, the expected release time of the drug solution can be adjusted over a wide range, depending on the density of the test solution to water.

The ommaya-like device of the system allows the delivery of drugs directly into the CSF and is especially useful for delivering oligonucleotides and antisense oligonucleotide drugs such as OT-101. Either alone or in combination with other cancer therapy.

In some aspects, the drug load may be surprisingly delivered as a single bolus infusion, or as a short infusion of 15, 30, or 60 min, and still achieve sustained delivery by the advantageous ratio of reservoir volume to catheter opening and/or the adjusted ratio of CSF density to drug solution density.

### Example 2

Examples of use of device for continuous infusion by intrathecal or intraventricular routes are shown in Tables 4 and 5. This formulations in this example provided effective use of the agents because the formulations were sufficiently stable to be delivered by continuous administration to patients. Formulations of this example contained minimal to no excipients and were stable for at least 14 days without fouling. Formulations of this invention were superior to conventional formulations which use ingredients such as LNP particles and excipients such as lactose. Formulations of this invention had improved stability and reduced bacterial growth as compared to such conventional formulations.

TABLE 4
Compatibility studies of OT-101 with device

| Study Name | In-use Conditions | Outcome of the Study |
|---|---|---|
| Compatibility of the Drug | Drug solution : 10 μM (61.43 ug/mL) OT- | The Drug Delivery System is |
| Delivery System with | 101 in isotonic saline | suitable for its intended use |
| 10 μM OT-101 Drug | Flow rate: 4 μL/min (corresponds to 5.76 | with regard to the |
| Solution | mL/day) | compatibility with a 10 μM |
| | Storage of the pump (including drug | OT-101 Drug Solution |
| | reservoir) and the non implanted parts of the | |
| | drug delivery system at ambient temperature | |
| | Storage of the implanted parts of the drug | |
| | delivery system at 37° C. | |
| | Drug reservoir content: 50 mL | |
| | Duration of test: 8 days | |
| Compatibility Study of the | The conditions are same as above except the | The Drug Delivery System is |
| Drug Delivery System for | external component of the Drug Delivery System | suitable for use in Climatic |
| Climatic Zone III & IV | kept at 30° C. to mimic the clincally relevant | Zone III/IV |
| | Climatic Zone III/IV | |
| Stability of OT-101 | Drug Conc.: 10 μM (61.43 μg/mL) | Based on the results OT-101 |
| Solutions at Different | Temp. 5° C. and 37° C., Diluent 0.9% NaCl, | of 10 μM in NaCl at 5° C. and |
| Temperatures | Container 6R Sample Vials, Duration 2 | 37° C. and OT-101 solution in |
| | weeks | |
| | Drug Conc.: 1 mg/mL | WFI at 5° C. are stable for at |
| | Temp. 5° C., Diluent WFI, Container 6R | least two weeks |
| | Sample Vials, Duration 2 weeks | |
| Compatibility Study of the | Drug solution: 15 mg/mL | None of the components |
| Drug Delivery Systems | Flow rate: 1 mL/h (corresponds to 24 | tested had impact on the |
| intended use at high | mL/day) | quality of the delivered Drug |
| concentration | Storage of the pump (including drug | Solution under in-use |
| | reservoir) and the non implanted parts of the | conditions. Based on the result |
| | drug delivery system at 30° C. | all Drug Delivery Systems |
| | Storage of the implanted parts of the drug | composed of any combination |
| | delivery system at 37° C. | of one of the tested pumps are |
| | Drug reservoir content: 120 mL | considered suitable with |
| | Duration of test: 5 days | regard to their compatibility |
| | | with OT-10 Drug solution |

TABLE 5
In use Stability Studies Performed

| Study Name | In-use Conditions | Outcome of the Study |
|---|---|---|
| Stability of Concentrated OT- | Drug Conc.: 7.35 mg/mL and 25 mg/mL | Based on the results from |
| 101 Drug Solutions | Temp. 5° C. and 37° C. | the study concentrated OT- |
| | Diluent 0.9% NaCl | 101in NaCl is stable for at |
| | Container 6R Sample Vials, | least two weeks |
| | Duration 2 weeks | |
| Sterility Testing of a OT-101 | Drug Conc.: 18.23 mg/mL | The results of this study |
| Drug Solution after 7 Days | Temp. 20-25° C. | demonstrate that the Cadd |
| Storage within the Cadd | Diluent 0.9% NaCl | Medication Cassette |
| Medication Cassette Reservoir | Container: Cadd Medication Cassette | Reservoir warrants sterility |
| | Reservoir | of a sterile filled drug |
| | Duration: 7 days | solution for a period of at |
| | | least 7 days. |

Studies A) to C) below were performed as in-use tests under conditions mimicking the clinical use for AP 12009 (OT-101)-P002. The treatment follows the similar set up of pump and reservoir as that of OT-101-P002. The Drug Delivery System suitable for this application is composed of the following medical device components:
Portable pump with corresponding drug reservoir and extension line
Gripper needle
Implantable port system with venous catheter

The three different Drug Delivery Systems were run in for in-use tests and those components intended to be implanted for clinical use being incubated at 37° C. and the non-implanted components being kept at ambient temperature. The three Drug Delivery Systems that have been tested for in-use stability of the OT-101 drug solution is described below in Table 6, Table 7, and Table 8.

TABLE 6
Drug Delivery System 1

| Description | Manufacturer | Ref. No. | Lot. No. | 510(k) |
|---|---|---|---|---|
| Cadd Legacy | Smith Medical | 21-630 | 230242 | K982839 |
| PCA Pump | MD Inc. USA | | | |
| 250 mL Reservoir | Smith Medical | 21-7308-24 | 408 × 10 | K990083 |
| for Cadd Pump | MD Inc. USA | | | |
| Cadd Extension Set | Smith Medical | 21-7106-24 | 256 × 50 | K040636 |
| | MD Inc. USA | | | |
| Huberplus 20 G | Bard/Juka | 012034 | D011718 | K993848 |
| | Pharma | | | |
| BardPort | Bard/Juka | 0602240 | REUG1127 | K153359 |
| | Pharma | | | |

TABLE 7
Drug Delivery System 2

| Description | Manufacturer | Ref. No. | Lot. No. | 510(k) |
|---|---|---|---|---|
| WalkMed 350 Pump | McKinley Medical, | 203190 | M7508 | K991275 |
| | USA | | | |
| Pump Reservoir for | WalkMed Infusion | IPR-150/10 | 2020503 | K870524 |
| WalkMed Infusion | LLC, USA | | | |
| Pump 150 mL | | | | |
| Pump Tubing Set for | WalkMed Infusion | EFV-101B/10 | 2109502 | NA* |
| WalkMed Infusion Pump | LLC, USA | | | |
| Intrastick System G20 | Fresenius Kabi | 8180611 | 33022131 | NA |
| Intraport CP | Fresenius Kabi | 8086001 | 33505120 | NA |

*Not available

TABLE 8
Drug Delivery System 3

| Description | Manufacturer | Ref. No. | Lot. No. | 510(k) |
|---|---|---|---|---|
| ambIT PCA Pump | Sorensoon Medical | 220262 | 116957 | K162165 |
| | Products Inc., USA | | | |
| Pump Reservoir for | WalkMed Infusion | IPR-150/10 | 2020503 | K870524 |
| WalkMed Infusion | LLC, USA | | | |
| Pump 150 mL | | | | |
| Cassette for ambIT | Summit Medical | 220266 | D016007 | NA* |
| | Products Inc., USA | | | |
| 0.2 μm Sterifilter | Smith Medical MD | F62 | 100008 | NA |
| | Inc., USA | | | |
| Gripper Needle | Smith Medical MD | 21-2714-24 | 265 × 37 | K870866 |
| | Inc., USA | | | |
| Sitimplant | Vygon | 8316.016 | 150710EA | NA |
| Titan Port | | | | |

*Not available

The conditions for the studies were:
Drug solution concentration: 15 mg/mL (calculated based on a mean dosage of 195 mg/m²/d and mean body surface of 1.85 m²)
Flow rate: 1 mL/h (corresponds to 24 mL/day)
Storage of the pump (including drug reservoir) and the non implanted parts of the drug delivery system at ambient temperature
Storage of the implanted parts of the drug delivery system at 37° C.
Drug reservoir content: 120 mL
Duration of test: 5 days

Study A: The delivered drug solution was sampled once a day and analyzed by means of two validated, stability indicating HPLC-methods (Ion Exchange and Reverse Phase HPLC). The results are summarized in Tables 9 and 10. The impurity profile of the pumped solution is considered to be adequate, if it meets the release specifications of AP 12009 (OT-101) 250 mg Drug Product. All samples meet the release specifications for AP 12009 (OT-101) 250 mg Drug Products with regard to the HPLC impurity profile. No relevant impact on the Drug Solution composition was observed after the passage of the different Drug Delivery Systems.

TABLE 9
Ion Exchange HPLC analysis

| | Results of Ion Exchange HPLC Analysis in Area (%) | | | | |
|---|---|---|---|---|---|
| | | AP 12009 | 3′N-2/PO*⁾ | N-1*⁾ | Total Other Imp. |
| Acceptance Criteria | | ≥90 | ≤4.8 | ≤4.2 | Report |
| Reference | t = 0 d | 97.13 | 1.78 | 0.89 | 0.20 |
| Drug | t = 1 d | 97.07 | 1.71 | 0.98 | 0.24 |
| Delivery | t = 2 d | 97.15 | 1.67 | 0.99 | 0.19 |
| System 1 | t = 3 d | 97.07 | 1.77 | 0.96 | 0.20 |
| | t = 4 d | 97.06 | 1.71 | 1.01 | 0.21 |
| | t = 5 d | 97.07 | 1.76 | 0.90 | 0.27 |
| Drug | t = 1 d | 97.03 | 1.74 | 0.97 | 0.26 |
| Delivery | t = 2 d | 97.07 | 1.71 | 0.99 | 0.23 |
| System 2 | t = 3 d | 97.04 | 1.75 | 1.00 | 0.21 |
| | t = 4 d | 97.12 | 1.73 | 0.89 | 0.26 |
| | t = 5 d | 97.14 | 1.79 | 0.88 | 0.20 |
| Drug | t = 1 d | 97.03 | 1.79 | 0.93 | 0.25 |
| Delivery | t = 2 d | 97.00 | 1.80 | 0.97 | 0.24 |
| System 3 | t = 3 d | 97.02 | 1.78 | 1.00 | 0.21 |
| | t = 4 d | 96.96 | 1.82 | 1.01 | 0.21 |
| | t = 5 d | 96.97 | 1.80 | 1.03 | 0.20 |

*⁾PO = impurity with one phosphorothioate moiety replaced by phosphate moiety (coeluting with 3′ N-2)
3′N-2 = impurity missing two 3′-terminal nucleotides (coeluting with PO)
N-1 = impurity missing the 3′ or 5′-terminal nucleotide
n.d. = not detected

TABLE 10
Reverse Phase HPLC Analysis
Results of Reverse Phase HPLC Analysis in Area (%)

| | | AP 12009 | 3′N-2*⁾ | 3′N-1*⁾ | 5′N-1/PO*⁾ | CNET*⁾ | Total Other Imp |
|---|---|---|---|---|---|---|---|
| Acceptance Criteria | | ≥85 | ≤0.6 | ≤3.4 | ≤7.6 | ≤5.2 | Report |
| Reference | t = 0 d | 96.35 | 0.10 | 0.81 | 2.28 | 0.30 | 0.17 |
| Drug Delivery System 1 | t = 1 d | 95.73 | 0.13 | 0.81 | 1.90 | 0.65 | 0.69 |
| | t = 2 d | 95.85 | 0.13 | 0.81 | 1.77 | 0.65 | 0.76 |
| | t = 3 d | 95.95 | 0.12 | 0.80 | 1.78 | 0.64 | 0.66 |
| | t = 4 d | 96.52 | 0.12 | 0.86 | 1.73 | 0.47 | 0.39 |
| | t = 5 d | 96.67 | 0.11 | 0.86 | 1.70 | 0.44 | 0.42 |
| Drug Delivery System 2 | t = 1 d | 96.25 | 0.11 | 0.96 | 2.22 | 0.26 | 0.20 |
| | t = 2 d | 96.39 | 0.10 | 0.86 | 2.17 | 0.28 | 0.20 |
| | t = 3 d | 96.59 | 0.10 | 0.70 | 2.15 | 0.26 | 0.20 |
| | t = 4 d | 96.56 | 0.10 | 0.94 | 1.88 | 0.32 | 0.21 |
| | t = 5 d | 96.54 | 0.11 | 0.82 | 2.02 | 0.29 | 0.23 |
| Drug Delivery System 3 | t = 1 d | 96.76 | 0.11 | 0.82 | 1.82 | 0.31 | 0.18 |
| | t = 2 d | 96.46 | 0.10 | 0.90 | 1.96 | 0.33 | 0.23 |
| | t = 3 d | 96.43 | 0.12 | 0.94 | 2.06 | 0.27 | 0.16 |
| | t = 4 d | 96.56 | 0.13 | 0.87 | 1.95 | 0.33 | 0.16 |
| | t = 5 d | 96.29 | 0.12 | 0.91 | 2.24 | 0.28 | 0.17 |

*⁾PO = impurity with one phosphorothioate moiety replaced by phosphate moiety(coeluting with 5′ N-1)
CNET = impurity with a cyanoethyl-moiety added to one of the thymidine nucleotide
3′N-2 = impurity missing two 3′-terminal nucleotide
3′N-1 = impurity missing the 3′-terminal nucleotide
5′N-1 = impurity missing 5′-terminal nucleotide (coeluting with PO)
n.d. = not detected

Study B: The delivered drug solution was sampled once a day and analyzed by UV-Spectroscopy. Acceptance criteria were set to 90%-110% of the initial sample concentration (Reference t=0 days) of the UV₂₆₀ₙₘ absorbance of the reference sample. Furthermore for all samples UV-spectra in the range of 230-320 nm were taken and compared to the spectrum of the reference sample.

The results are listed in Table 11 and show that all sample concentrations were within the range of 100.0%-102.7% of the reference solution and met the acceptance criteria of 90-100%.

TABLE 11
UV Analysis

| | | |
|---|---|---|
| Sample ID | A_{260 nm} | Concentration (%) |

| | | | |
|---|---|---|---|
| Reference | t = 0 d | 0.6157 | 100.00 |
| Drug | t = 1 d | 0.6254 | 101.58 |
| Delivery | t = 2 d | 0.6198 | 100.67 |
| System 1 | t = 3 d | 0.6245 | 101.43 |
| | t = 4 d | 0.6240 | 101.67 |
| | t = 5 d | 0.6329 | 102.79 |
| Drug | t = 1 d | 0.6226 | 101.12 |
| Delivery | t = 2 d | 0.6275 | 101.92 |
| System 2 | t = 3 d | 0.6217 | 100.97 |
| | t = 4 d | 0.6270 | 101.84 |
| | t = 5 d | 0.6322 | 102.68 |
| Drug | t = 1 d | 0.6204 | 100.76 |
| Delivery | t = 2 d | 0.6190 | 100.54 |
| System 3 | t = 3 d | 0.6219 | 101.01 |
| | t = 4 d | 0.6212 | 100.89 |
| | t = 5 d | 0.6280 | 102.00 |

Study C: The delivered drug solution was sampled at the end of the in-use test. The leachable/extractable profile was determined by a contract laboratory using gas chromatography based analyses established for chemical analysis of medical devices according to ISO 10993 (i.e. quantification of released organic compounds using a flame ionization detector (GC-FID) and identification of potential leachables/extractables using gas chromatography mass spectroscopy (GC-MS) and comparison with the NIST/EPA/NIH 2005 Mass Spectral Library, if applicable). The samples were sent to the contract lab Medical Device Services along with the reference sample (t-0 d). Due to incompatibility of saline with gas chromatography, the samples were extracted with tert-butyl-ether and these extracts were then exposed to gas chromatography. No leachables/extractables were detected as per MDS report #111523-20.

Study D: The clinical study AP 12009-P002 will use two dosages, which are 140 mg/m²/d and 250 mg/m²/d. A representative drug solution concentration to be used in this test was calculated assuming a body surface of 1.75 m² and a dosage of 250 mg/m²/d, shown in Table 12.

TABLE 12
Drug parameters

| | | |
|---|---|---|
| Amount of Trabedersen | 1750 | mg |
| (250 mg/m²/d × 1.75 m² × 4 d): | | |
| Volume required for 4 days | 96 | mL |
| of continuous infusion: | | |
| Drug solution concentration: | 18.23 | mg/mL |

This representative drug solution was prepared in duplicate and filled into two separate Cadd Medication Cassette Reservoirs under aseptic conditions. The Cadd Medication Cassette Reservoirs were stored within an incubation chamber (unsterile setting) at a temperature of 20° C.-25° C. The temperature of this incubation chamber was continuously monitored.

After the storage period of 7 days Cadd Extension Set infusion lines were connected to the luer-lock connectors of the reservoir bags under aseptic conditions. 20 mL of the incubated drug solutions were removed via the extension sets (including a 0.22 m sterile filter) into separate sterility test equipment. Another 20 ml of the incubated drug solutions were removed directly from the reservoir bag into separate sterility test equipment by means of sterile cannulae. The results of the sterility tests are shown in Table 13.

TABLE 13
Sterility Test Results

| | Incubation Time of | |
|---|---|---|
| | Drug Solution within | |
| Description | the Drug Reservoirs | Result |
| Incubated Drug | 7 days | sterile |
| Solution removed | | |
| directly | | |
| Incubated Drug | 7 days | sterile |
| Solution removed | | |
| via sterile filter | | |
| Incubated Drug | 7 days | sterile |
| Solution removed | | |
| directly | | |

The results of this study demonstrate that the Cadd Medication Cassette Reservoir warrants sterility of a sterile filled drug solution for a period of at least 7 days.

Dosing Solution of Concentration 10 μM (61.43 μg/mL).

Compatibility of the Drug Delivery System for the Clinical Study AP 12009 (OT-101)-G005 with 10 μM OT-101 Drug solution was demonstrated by verification of the integrity of the said Drug Solution after passage of the Drug Delivery System under conditions mimicking its clinical use. Integrity of the Drug Solution was evaluated based on the following parameters: impurity profile, concentration, sterility and leachable/extractable profile.

Compatibility of the OT-101 drug solution with the intended drug delivery system was demonstrated by the following studies:
a) Determination of the Impurity Profile of the OT-101 Drug Solution after Passage of the Drug Delivery System
b) Verification of the Drug Solution Concentration after Passage of the Drug Delivery System
c) Determination of the Leachable/Extractable Profile of the OT-101 Drug Solution after Passage of the Drug Delivery System
d) Sterility Testing of a Drug Solution after Incubation within the Drug Reservoir

The Drug Delivery System suitable for this application is composed of the following medical device components:
Portable pump with corresponding drug reservoir and extension line
Gripper needle
Implantable port system with venous catheter

The Drug Delivery Systems were run in for in-use tests with those components intended to be implanted for clinical use being incubated at 37° C. and the non-implanted components being kept at ambient temperature.

The Drug Delivery Systems that has been tested for in-use stability of the OT-101 drug solution is described below in Table 14.

TABLE 14
Drug Delivery System

| Description | Manufacturer | Ref. No. |
|---|---|---|
| Pega Vario Infusion Pump | Venner Medical, | 30151 E |
| Pega Bag 50 mL | Germany | 16050 |
| Pega Pump Head | | 10210 |
| Infusion Line incl. | | 10510 |
| Sterile Filter | | |
| Gripper Needle | Smith Medical, | 21-2714-24 |
| Cath-A-Port incl. | USA | 21-4034-24 |
| Port Catheter | | |
| Connector | Medronic, | 45103 |
| Ventricular Catheter | USA | 41207 |

Studies a) to c) were performed as in-use tests under conditions mimicking the clinical use. These conditions were:
Drug solution concentration: 10 μM (61.43 μg/mL) OT-101 in isotonic saline
Flow rate: 4 μL/min (corresponds to 5.76 mL/day)
Storage of the pump (including drug reservoir) and the non implanted parts of the drug delivery system at ambient temperature
Storage of the implanted parts of the drug delivery system at 37° C.
Drug reservoir content: 50 mL
   These tests were limited to a maximum duration of about 8.5 days, due to the drug reservoir volume of 50 mL and a flow rate of 5.76 mL per day.

Study a: The delivered drug solution was sampled once a day and analyzed by means of two validated, stability indicating HPLC-methods (Ion Exchange and Reverse Phase HPLC). Acceptance criteria were set to two times the impurity area % of the respective impurity in the Tox-Batch (J982-15K9FP). In addition, warning limits were set based on the release specifications of OT-101 (AP 12009) 7.37 mg drug product. Based on the impurity data shown in Table 15, the results demonstrate that during the in-use period (under clinically relevant conditions) only minor degradation was observed and that all samples met the drug product release specifications. The main trend observed was a slight increase of the PO-impurity. This is consistent with the forced degradation result, which had identified PO as the most pronounced degradation product. PO is an impurity in which one of seventeen phosphorothioate moieties is oxidized to a phosphate diester moiety. This impurity is discussed to be more a related product than an impurity (in terms of an inactive degradation product).

TABLE 15
Results of Impurity Profile Testing of the Drug solution
after Passage of the Drug Delivery System

| | Results of Ion Exchange HPLC | | | | Results of Reverse Phase | | | | | |
|---|---|---|---|---|---|---|---|---|---|---|
| | Analysis in Area (%) | | | | HPLC Analysis in Area (%) | | | | | |
| | | | | Total | | | | | | Total |
| | AP | | | Other | AP | | | | | Other |
| | 12009 | 3′N-2/PO*⁾ | N-1*⁾ | Imp. | 12009 | 3′N-2*⁾ | 3′N-1*⁾ | 5′N-1/PO*⁾ | CNET*⁾ | Imp |
| Acceptance Criteria | | ≤8.8 | ≤7.8 | | | ≤1.1 | <6.1 | ≤14.0 | ≤5.2 | |
| based on Tox-Batch | | | | | | | | | | |
| Warning Limits = IMP | ≥90 | ≤4.8 | ≤4.2 | Report | ≥85 | ≤0.6 | ≤3.4 | ≤7.6 | ≤5.2 | Report |
| Release Spec. | | | | | | | | | | |
| Reference (t = 0 d) | 97.61 | 1.29 | 1.12 | n.d. | 93.36 | 0.28 | 1.46 | 3.19 | 0.45 | 1.28 |
| t = 1 d | 95.87 | 3.14 | 1.00 | n.d. | 90.94 | 0.31 | 1.56 | 5.36 | 0.46 | 1.39 |
| t = 2 d | 95.50 | 3.45 | 1.06 | n.d. | 90.52 | 0.33 | 1.60 | 5.90 | 0.44 | 1.23 |
| t = 3 d | 95.58 | 3.39 | 1.04 | n.d. | 90.16 | 0.34 | 1.65 | 5.92 | 0.45 | 1.50 |
| t = 4 d | 95.69 | 3.32 | 1.00 | n.d. | 90.94 | 0.34 | 1.56 | 5.59 | 0.44 | 1.15 |
| t = 5 d | 94.81 | 4.16 | 1.04 | n.d. | 89.83 | 0.31 | 1.66 | 6.65 | 0.43 | 1.13 |
| t = 6 d | 94.63 | 4.27 | 1.11 | n.d. | 90.05 | 0.34 | 1.61 | 6.34 | 0.44 | 1.21 |
| t = 7 d | 95.05 | 3.86 | 1.09 | n.d. | 89.97 | 0.34 | 1.67 | 6.36 | 0.41 | 1.25 |
| t = 8 d | 95.00 | 4.00 | 1.01 | n.d. | 89.72 | 0.40 | 1.73 | 6.47 | 0.45 | 1.24 |

*⁾PO = impurity with one phosphorothioate moiety replaced by phosphate moiety
CNET = impurity with a cyanoethyl-moiety added to one of the thymidine nucleotide
3′N-2 = impurity missing two 3′-terminal nucleotide
3′N-1 = impurity missing the 3′-terminal nucleotide
5′N-1 = impurity missing 5′-terminal nucleotide (coeluting with PO)

Study b: The delivered drug solution was sampled once a day and analyzed by UV-Spectroscopy. Acceptance criteria were set to 90%-110% of the initial sample concentration (Reference t 0 days). This test was performed in duplicate. The results are listed in Table 16 and show that all sample concentrations were within the range of 98%-102% of the reference. These results demonstrate that the drug solution concentration was not affected during passage of the drug delivery system (e.g. no adsorption of the drug to polymeric surfaces of the drug delivery system).

TABLE 16
Results of UV-Spectroscopy of Drug Solutions
after Passage of the Drug Delivery System

| | Run 1 | | Run 2 | | |
|---|---|---|---|---|---|
| | A₂₆₀ mean | | A₂₆₀ mean | | Mean |
| | from 3 | Concentration | from 3 | Concentration | Concentration |
| | replicate | in relation to | replicate | in relation to | in relation to |
| | measurements | reference (%) | measurements | reference (%) | reference (%) |
| Reference | 0.5192 | 100.00 | 0.5192 | 100.00 | 100.00 |
| (t = 0 d) | | | | | |
| t = 1 d | 0.5200 | 100.15 | 0.5283 | 101.75 | 100.95 |
| t = 2 d | 0.5184 | 99.85 | 0.5147 | 99.13 | 99.49 |
| t = 3 d | 0.5145 | 99.09 | 0.5215 | 100.44 | 99.77 |
| t = 4 d | 0.5123 | 98.67 | 0.5176 | 99.69 | 99.18 |
| t = 5 d | 0.5194 | 100.04 | 0.5148 | 99.15 | 99.90 |
| t = 6 d | 0.5168 | 99.54 | 0.5222 | 100.58 | 100.06 |
| t = 7 d | 0.5139 | 98.98 | 0.5205 | 100.25 | 99.62 |
| t = 8 d | 0.5252 | 101.16 | 0.5235 | 100.83 | 101.00 |

Study c: The delivered drug solution was sampled at the end of the in-use test. The leachable/extractable profile was determined by a contract laboratory using gas chromatography based analyses established for chemical analysis of medical devices according to ISO 10993, i.e. quantification of released organic compounds using a flame ionization detector (GC-FID) and identification of potential leachables/extractables using gas chromatography mass spectroscopy (GC-MS) and comparison with the NIST/EPA/NIH 2005 Mass Spectral Library, if applicable. No leachables/extractables could be detected in this sample.

Taking both HPLC data of the 5 days across all in-use stability studies gave the following regression: Slope: −0.001065±0.0009457, with the time to 10% reduction in OT-101 concentration of 91 days. Thus, formulations of this invention were surprisingly stable for long periods in-use.

### Example 3

Pharmacokinetics after Intracerebral and Intraventricular Administration in Rats

Distribution, pharmacokinetics, and excretion of ³H-AP12009 were investigated after a single i.e. infusion into the frontal lobe of the cerebrum of Sprague-Dawley rats (10 μCi/rat, 87 μM solution, flow rate: 0.4 μL/min, administered over 1 hour; Report GAS0002). Due to the low sample volume applied, the small brain size, and variability in the exact location of the infusion device, there was a fairly wide inter-animal variability of the data for the early time points (0 to 4 hours after completion of infusion), both in terms of measured systemic radioactivity and tissue concentrations. Overall, trabedersen was readily distributed from the site of dosing to other regions of the brain, particularly the cerebrospinal fluid but also in the systemic circulation.

Intravenous dosing of OT-101 resulted in minimal accumulation in CNS. Concentration of radioactivity in blood and tissues following a single intravenous administration of [³H]OT-101 to male rats resulted in 0.1% of OT-101 in the brain, shown in Table 17.

TABLE 17
Concentration of radioactivity in blood and tissues
[³H]OT-101 Concentration (ng equivalent/g)

| Hrs | Blood | CSF | Brain | Kidney | Liver | Spleen |
|---|---|---|---|---|---|---|
| 1 | 30100 | 305 | 996 | 64200 | 23800 | 13900 |
| 4 | 3560 | 1800 | 421 | 107000 | 31500 | 11300 |
| 24 | 939 | 726 | 581 | 132000 | 22700 | 6430 |
| 72 | 662 | 687 | 528 | 109000 | 15400 | 5010 |

To improve the use of OT-101 therapeutic, intrathecal delivery of tritiated OT-101 into Sprague-Dawley CD (albino) rats was studied. Throughout the studies, there were no sex differences. After 1 hr intracerebral infusion in rats, OT-101 was limited to the infusion site. Whereas for the 1 hr intraventricular infusion, OT-101 was more widespread with 35×, 19×, 12× higher concentration found in cerebellum, remaining cerebrum, cerebrospinal fluid (CSF), respectively. OT-101 concentration was stable for the first 4 hours post infusion and decayed biexponentially with a slow terminal half life for tissue but not for CSF, suggestive of rapid penetration of the underlying tissue away from the CSF compartment. Minimum amount of OT-101 was detected in the plasma compartment. Intrathecal bolus administration of 0.1 mL of OT-101 at 14, 30, 200, 300, and 500 μM into cynomolgus monkeys did not result in any single dose toxicity. Histopathology examination revealed no substance-related histomorphological lesions in the cavum subarachnoideale of the lumbar region. No changes were noted in the grey and white matter of the spinal cord, the nerve trunk, and nerve cells did not show any abnormalities. These data show that intrathecal administration of OT-101 is a potentially effective delivery route for antisense therapeutics such as OT-101 to the midline, i.e. for Diffuse Midline Glioma (DMG).

Diffuse midline glioma (DMG) is a highly morbid pediatric central nervous system (CNS) tumor for which there is currently no effective treatment. DMG is responsible for 50% of all childhood HGG. Due to their anatomic location and infiltrative nature, DMGs are not amenable to surgical resection and are most often diagnosed radiographically and treated with radiation therapy, with no effect on survival. The median age at diagnosis is 5 to 11 years with tumors that arise in the pons occurring at a younger age (˜7 years) than those that arise in the thalamus (˜11 years). DMG patients face a very poor median overall survival (OS) of just 9-11-months, with <10% of patients with pontine tumors surviving two years post-diagnosis. Radiation remains the mainstay of therapy, though it is only palliative, and is expected to increase survival by an average of 3 months.

OT-101 antisense drugs are safe and effective during extended (7-day) high flow perfusion of the brain in adult gliomas. As single agent it is as effective as the most active drug in adult gliomas, TMZ for chemo naïve patients BCNU/CCNU in chemo failure patients.

TGF-β2, is expressed at high levels in both pediatric GBM (WHO Grade IV) and pediatric DIPG (WHO Grade IV) patients.

Expression analyses of pediatric brainstem cases of the TCGA database yielded highly significant survival benefit across all four quartiles of TGF-β2 expression.

Expression analyses of all gliomas cases treated with radiation yielded highly significant survival benefit across all quartiles of TGF-β2 expression.

### Intracerebral Infusion

Dosing by intracerebral infusion or intraventricular infusion resulted in distribution of OT-101 throughout the CNS. Dosing was done at 12 ug/rat. The distribution profiles between cerebral and ventricular administration were similar except for a higher drug concentration in cerebellum and CSF with direct administration into the CSF compartment with intraventricular administration. The similarity of intraventricular administration versus intracerebral administration surprisingly showed that intraventricular administration is equally effective as intracerebral administration, shown in Table 18.

TABLE 18
Dosing by intracerebral infusion or intraventricular infusion
[³H]OT-101 Concentration (ng equivalent/g) median (min-max)

| | | | | | |
|---|---|---|---|---|---|
| | Dose | | Remaining | Remaining | |
| Hrs | Site | Cerebellum | cerebrum | brain | CSF |

Intracerebral infusion (N = 8)

| | | | | | |
|---|---|---|---|---|---|
| 0 | 30400 | 1320 | 1360 | 3135 | 13350 |
| | (20648-38800) | (430-1913) | (997-2618) | (2358-3910) | (3308-24325) |
| 1 | 12850 | 851 | 1610 | 4310 | 2285 |
| | (9950-14825) | (345-1453) | (948-4384) | (2815-5393) | (1420-4325) |
| 4 | 18400 | 692 | 2565 | 1883 | 1056 |
| | (5800-30175) | (545-1045) | (990-3341) | (934-3610) | (610-1185) |
| 24 | 7820 | 432 | 962 | 1141 | 124 |
| | (4504-11850) | (273-464) | (696-1703) | (909-1775) | (89-140) |
| 48 | 3145 | 165 | 375 | 287 | 24 |
| | (2562-4150) | (39-227) | (190-569) | (107-498) | (15-29) |

Intraventricular infusion (N = 1)

| | | | | | |
|---|---|---|---|---|---|
| 1 | 30875 | 7938 | 2144 | 2944 | 5844 |
| 4 | 22250 | 1531 | 1069 | 2138 | 2094 |
| 24 | 2450 | 1413 | 284 | 534 | 71 |
| 48 | 850 | 401 | 250 | 317 | 26 |

The accumulation profiles for the top 15 organs are shown below. At the end of the infusion period of 1 hr, the rapidly perfused CNS components readily accessible by CSF (dose site, cerebellum, CSF, pituitary gland) achieved Cₘₐₓ. Though readily accessible to CSF, the pineal organ actively taken up OT-101 and did not achieve Cₘₐₓ until 4 hr. The less perfused CNS components achieved Cₘₐₓ at 1 (remaining cerebrum, remaining brain) and 4 hr (spinal cord).

A small fraction of administered OT-101 reached the plasma compartment with Cₘₐₓ at 1 hr. The organs readily perfused by blood achieve Cₘₐₓ at 4 hr timepoint (thyroid and liver) and those actively taken up OT-101 did not achieved Cₘₐₓ until 24 hr (kidney, spleen, bone marrow) or 72 hr (thymus), shown in Table 19.

TABLE 19
Dosing by intracerebral infusion or intraventricular infusion
Concentration of radioactivity in tissues following intracerebral
administration of [³H]OT-101 at dose level
of 12 ug/rat. Concentration = ng equivalent/g (mean, SD,
N) Infusion time = 1 hr.

| Hours (post infusion) | 0 | 1 | 4 | 24 | 72 |
|---|---|---|---|---|---|
| Dose Site | 28165 | 12929.69 | 23018.36 | 8449.063 | 3025.68 |
| | 12950.03 | 5534.462 | 21306.78 | 5105.702 | 1426.363 |
| | 6 | 8 | 8 | 8 | 8 |
| Cerebellum | 1239.167 | 1238.359 | 847.5945 | 457.5092 | 141.8422 |
| | 790.1148 | 1419.591 | 679.9536 | 363.2347 | 93.60506 |
| | 6 | 8 | 8 | 8 | 8 |
| Remaining | 1683 | 2297.609 | 2215.538 | 1062.155 | 381.9844 |
| cerebrum | 816.6407 | 2121.81 | 1380.145 | 654.7472 | 247.8907 |
| | 6 | 8 | 8 | 8 | 8 |
| Remaining | 3056.833 | 5108.281 | 2020.409 | 1291.969 | 303.3984 |
| brain | 1219.287 | 3511.84 | 1436.072 | 468.2549 | 214.2532 |
| | 6 | 8 | 8 | 8 | 8 |
| CSF | 14481.67 | 3688.516 | 916.2813 | 118.6641 | 20.72031 |
| | 11839.01 | 4316.185 | 472.7928 | 51.46708 | 10.8167 |
| | 6 | 8 | 8 | 8 | 8 |
| Pituitary | 129.15 | 109.2833 | 58.7 | 61.96667 | 9.793333 |
| Gland | 97.75291 | 120.1135 | 43.55865 | 46.16759 | 7.387562 |
| | 6 | 6 | 6 | 6 | 6 |
| Spinal | 255.5117 | 401.6667 | 403 | 175.25 | 70.41667 |
| Cord | 212.2003 | 327.0056 | 135.6112 | 83.68975 | 56.14148 |
| | 6 | 6 | 6 | 6 | 6 |
| Thyroid | 48.19167 | 29.35167 | 42.455 | 16.15 | 9.025 |
| | 65.89372 | 28.18978 | 57.86549 | 5.318928 | 3.068288 |
| | 6 | 6 | 6 | 6 | 6 |
| Kidneys | 16.41 | 102.9667 | 221.4333 | 368.6667 | 155.5833 |
| | 7.602187 | 93.99691 | 295.9934 | 168.7539 | 67.75132 |
| | 6 | 6 | 6 | 6 | 6 |
| Bone | 3.243333 | 9.605 | 19.405 | 49.71667 | 31.63333 |
| Marrow | 1.282336 | 8.159686 | 20.65162 | 11.29751 | 14.77656 |
| | 6 | 6 | 6 | 6 | 6 |
| Liver | 5.396667 | 17.44667 | 28.82167 | 26.05 | 9.445 |
| | 2.392201 | 14.68127 | 41.37177 | 17.74528 | 2.691875 |
| | 6 | 6 | 6 | 6 | 6 |
| Plasma | 8.85 | 16.015 | 14.60167 | 12.86667 | 6.886667 |
| | 4.799429 | 10.76895 | 6.639308 | 0.877876 | 1.750448 |
| | 6 | 6 | 6 | 6 | 6 |
| Pineal | 609.6667 | 1255 | 1317.333 | 1045.833 | 403.3833 |
| body | 549.5157 | 822.2618 | 1440.409 | 1804.737 | 542.7811 |
| | 6 | 6 | 6 | 6 | 6 |
| Spleen | 3.325 | 15.045 | 8.606667 | 22.25 | 15.73833 |
| | 1.386243 | 17.80838 | 2.688372 | 7.181017 | 4.627355 |
| | 6 | 6 | 6 | 6 | 6 |
| Thymus | 2.531667 | 4.065 | 5.318333 | 13.38333 | 13.92333 |
| | 0.836144 | 1.33614 | 1.38293 | 2.050772 | 5.425649 |
| | 6 | 6 | 6 | 6 | 6 |

The data from this distribution study clearly showing that the entire CNS including the midline is accessible to OT-101 by intracerebral infusion (and therefore intraventricular infusion). The profile is consistent with CSF flow and location of various compartments examined. CNS components with high level of OT-101 are readily accessible to the CSF including the pituitary gland and pineal body.

OT-101 has been subjected to a series of nonclinical safety and pharmacology studies. The salient features of the conclusions from these studies were as follows:

Prolonged local administration of OT-101/AP 12009 may result in local tissue inflammation There was mild to moderate local toxicity observed in animals after infusion of a concentration of 500 μM without any macroscopic changes.

When administered to 3 kg male or female rabbits as a bolus IT injection at a 0.12 mg/kg dose level (500 μM solution; 0.1 mL) with an estimated CSF concentration of 4.16 μM, OT-101 did not cause any clinical toxicity or drug-related macroscopic/microscopic changes consistent with sub-clinical toxicity.

When administered to 6-7 kg cynomolgus monkeys as a bolus IT injection at a 0.05 mg/kg dose level (500 μM solution; 0.1 mL) with an estimated CSF concentration of 0.46 μM, OT-101 did not cause any clinical toxicity or drug-related macroscopic/microscopic changes consistent with sub-clinical toxicity.

When given to rats via intraventricular administration, radiolabeled OT-101 (0.18 mg/kg) was detected within 1 hour after administration not only in the CSF but also in the cerebrum, cerebellum and pineal body. The CSF and brain tissue half-lives were <24 hours with <15% residual OT-101 remaining at 72 hours.

The intratumoral placement of the catheter posed potential risks avoidable with the use of an ommaya reservoir to access the ventricular space.

The ommaya reservoir can be used in the treatment of leptinomenigeal cancers (LM) from multiple malignant tumors.

### Example 4

FIG. 9 shows the effect of delivering a pharmaceutical composition by intrathecal or intraventricular continuous infusion. FIG. 9 shows the effect of OT-101 Treatment on TGF-β2 Secretion from the Human GBM cell line A-172. Cells were incubated with the indicated different concentrations of OT-101/AP 12009 (1 μM to 80 μM) for 7 days. Secreted TGF-β2 was measured in cell supernatants by ELISA. Results represent median, minimum, and maximum values from 3 independent experiments.

### Example 5

Example of Continuous Infusion with a Partially Collapsible Ommaya-Like Reservoir of this Invention as Compared to Conventional Direct Injection Methods.

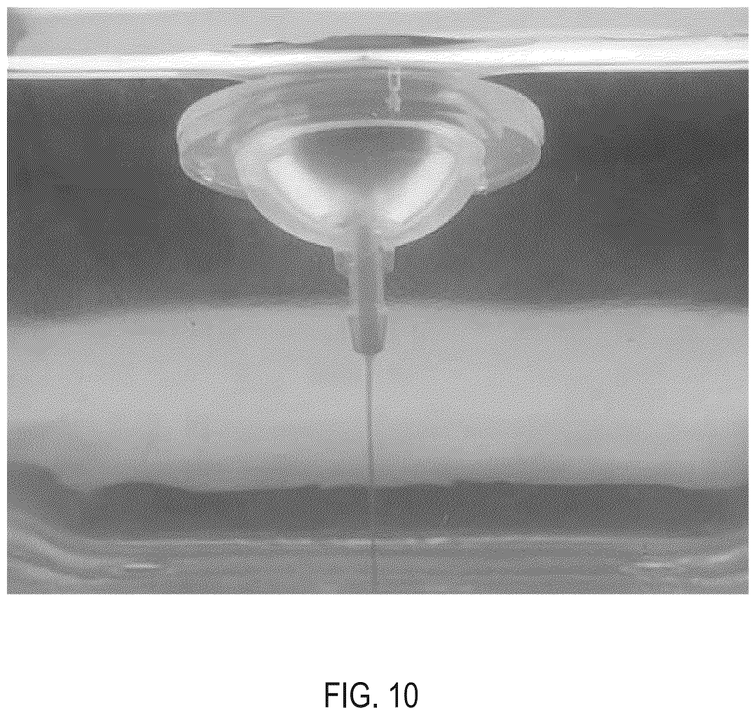

A device and method of this invention for continuous infusion of a therapeutic solution was found to be advantageous as compared to conventional methods (FIG. 10).

In conventional methods, a therapeutic drug solution is directly injected into the ventricular space of a patient's brain, or directly injected into the ventricular space via a catheter. In these conventional methods, the therapeutic solution is rapidly dispersed into the ventricular space in seconds to minutes. Further, such rapidly injected therapeutic solution is rapidly cleared by the brain tissue. Thus, such conventional methods achieve very little therapeutic effect because of the limited exposure of the brain tissue to the therapeutic solution.

By comparison, an ommaya-like reservoir of this invention having a partially collapsible top advantageously provided continuous infusion over an extended period of time, essentially continuous infusion.

The ommaya-like reservoir of this invention was filled with a test solution. The device was placed on a water surface in a tank to test the rate of infusion, i.e. the rate at which the test solution flowed out of the catheter end tip, as a model for continuous infusion into brain.

In one embodiment, the ommaya-like reservoir of this invention had a surface area of 339 mm², and the area of the inner opening of the catheter was 1.5386 mm², which provided a ratio of 339/1.5386=220. With this ratio, the expected release time of the test solution would be at least several hours, depending on the density of the test solution to water.

In this example, the test solution diffused slowly out through the catheter opening (see FIG. 10). The diffusion from the ommaya-like reservoir of this invention occurred continuously over 4 hours. This test showed that continuous diffusion was achieved with test solution, and that a therapeutic solution having a density adjusted or matched to CSF would provide continuous infusion over a period of at least one to several days.

This example showed that the partially-collapsible design of the ommaya-like reservoir of this invention is advantageous because when infusion of a therapeutic solution is continuous over an extended period of time, the accidental release of therapeutic solution by accidental or incidental pressure on the ommaya-like reservoir flexible top must be prevented. For example, accidental or incidental pressure on the ommaya-like reservoir flexible top could release far too much of the therapeutic solution in a very short time.

The partially-collapsible design of the ommaya-like reservoir of this invention prevents such unwanted accidental or incidental releases of the therapeutic solution.

Furthermore, the partially-collapsible design of the ommaya-like reservoir of this invention allows the device to maintain fluid flow and movement into, and out of the reservoir. The flexible top expands and contracts in response to pressure changes, which allows the ommaya-like reservoir of this invention to breath properly for continuous flow of the solution, especially in the presence of externally generated pumping action. Unwanted accidental or incidental release can also occur due to patient movements, for example, movement of the patient's jaw and other muscles.

In some examples and embodiments, an ommaya-like reservoir of this invention is prepared with a collar making it impossible to fully collapse the reservoir and preventing accidental or incidental release of solution (see FIG. 8).

In some examples and embodiments, an ommaya-like reservoir of this invention is prepared partially-collapsible by making a portion of the top of the ommaya-like reservoir from a hard, inflexible plastic.

### Example 6

### Pediatric Diffuse Intrinsic Pontine Glioma.

In this application, methods for pediatric DMG/DIPG/or K27M GBM are disclosed.

In this example, new evidence is shown that amplified expression of TGFB2 mRNA in pediatric DIPG and H3K27M-mutant GBM is correlated with upregulated mRNA expression for several transcription factors/DNA binding proteins that are known to augment TGFB2 gene expression. This example provides the first evidence that high level TGFB2 mRNA expression is associated with a poor treatment outcome in DIPG. The reported results also support the notion that further evaluation of the clinical potential of new strategies targeting TGFB2 mRNA in pediatric DIPG is warranted.

The median survival for TGFB2high patients within the 30-patient H3K27M-mutant subset was 6.5 months (95% CI=5-NA months, 6 events, N=8) which was significantly shorter than the median survival for the remaining patients (Median median >10 months, 7 events, N=22);

Pediatric diffuse intrinsic pontine gliomas (DIPGs) are one of the most aggressive and deadliest childhood brain tumors. The purpose of the present study was to evaluate the clinical significance of amplified expression levels of transforming growth factor beta 2 (TGFB2) in the tumor tissue specimens from DIPG patients. Our findings provide the first evidence that high level TGFB2 expression is associated with a poor treatment outcome in DIPG. The reported results also support the notion that further evaluation of the clinical potential of new strategies targeting TGFB2 in pediatric DIPG is warranted.

Tumor samples from newly diagnosed pediatric diffuse intrinsic pontine glioma (DIPG) patients express significantly higher levels of transforming growth factor beta 2 (TGFB2, also TGF-β2) messenger ribonucleic acid (mRNA) than control pons samples, which correlated with augmented expression of transcription factors that upregulate TGFB2 gene expression. Our study also demonstrated that RNA sequencing (RNAseq)-based high TGFB2 mRNA level is an indicator of poor prognosis for DIPG patients, but not for pediatric glioblastoma (GBM) patients or pediatric diffuse midline glioma (DMG) patients with tumor locations outside of the pons/brainstem. Notably, DIPG patients with high levels of TGFB2 mRNA expression in their tumor samples had significantly worse overall survival (OS) and progression-free survival (PFS). By comparison, high levels of transforming growth factor beta 3 (TGFB3) mRNA expression in tumor samples was associated with significantly better survival outcomes of DIPG patients, whereas high levels of transforming growth factor beta 1 (TGFB1) expression was not prognostic. Our study fills a significant gap in our understanding of the clinical significance of high TGF expression in pediatric high-grade gliomas

Pediatric diffuse intrinsic pontine gliomas (DIPGs) belong to the most aggressive and deadliest childhood brain tumors classified as histone H3 Lysine 27 (H3K27)-altered diffuse midline gliomas (DMG), including the H3 K27-mutant as well as H3 wildtype subtypes with overexpression of the EZH inhibitory protein (EZHIP). Complete resection of DIPGs, which are the second most common malignant pediatric brain tumors, is not possible due to its anatomic location in the brainstem and its rapid infiltrative growth. DIPG is associated with a poor overall survival (OS) despite contemporary radiation therapy/radio-chemotherapy strategies, and it is one of the driving contributors to cancer-related mortality in children (median OS<1 year). In spite of numerous clinical trials of chemotherapeutic agents, immune-oncology drugs, and specific targeted therapies aimed at improving the survival outcome of pediatric patients with DIPG, little progress has been achieved and the prognosis of DIPG remains dismal, with a median survival time of approximately 10 months, and a two-year survival rate of less than 10 percent.

We present new evidence that amplified expression of TGFB2 mRNA in pediatric DIPG and H3K27M-mutant GBM is correlated with upregulated mRNA expression for several transcription factors/DNA binding proteins that are known to augment TGFB2 gene expression. Our findings provide the first evidence that high level TGFB2 mRNA expression is associated with a poor treatment outcome in DIPG. The reported results also support the notion that further evaluation of the clinical potential of new strategies targeting TGFB2 mRNA in pediatric DIPG is warranted.

Furthermore, there is no standard treatment for progressive DIPG after the failure of frontline radiation therapy, and no salvage regimen has been shown to extend the OS. Effective treatment strategies that could potentially improve the dismal prognosis of these children are urgently needed and their discovery represents a main focus of translational and clinical research in contemporary neuro-oncology. Several interventional strategies are being explored, such as immunotherapy with immune-checkpoint inhibitors or T cells with a chimeric antigen receptor (CAR) (“CAR-T cells”), inhibition of signal transduction pathways with small molecule drugs, and biotherapy with fusion toxins administered via convection-enhanced delivery (CED) or oncolytic viruses.

Transforming growth factor-beta (TGFB) is a disulfide-linked, homodimeric cytokine with a pleiotropic activity profile that has been implicated in oncogenesis as well as suppression of host anti-tumor immunity within the tumor microenvironment (TME). Augmented transforming growth factor beta (TGFB) signaling pathway activity mediated by autocrine or tumor-associated macrophages (TAM)-derived overproduction has been implicated in the aggressive biology and poor overall survival of adult high-grade glioma patients by promoting invasive and rapid glioma cell growth. The TGFB pathway has also been shown to contribute to a “cold” TME in high-grade gliomas via its immunosuppressive effects characterized by inhibition of CD8-antigen-positive cytotoxic T cells, natural killer (NK) cells, and activation of regulatory T cells (Tregs) as well as myeloid derived suppressor cells (MDSCs). The TGFB pathway has emerged as a possible therapeutic target for high-grade gliomas. The FDA-approved TGFB inhibitor Pirfenidone (5-methyl-1-phenyl-2(1H)-pyridone, PFD) has been shown to inhibit TGFB expression in malignant glioma cells. A synthetic TGFB2 mRNA-targeting anti-sense phosphorothioate oligodeoxynucleotide (S-ODN) exhibited promising single-agent clinical activity associated with durable complete and partial responses in adult patients with recurrent or refractory glioblastoma and anaplastic astrocytoma when administered intratumorally via CED. In a study using a microarray-based gene expression platform, it was found that TGFB2 mRNA levels were selectively amplified in primary tumor samples from 29 pediatric DIPG patients compared to normal samples and primary tumor samples from low-grade glioma patients.

The present study evaluated the clinical prognostic significance of high tumor TGFB2 mRNA levels in pediatric DIPG, as measured by RNAseq. Pediatric DIPG patients who have amplified TGFB2 mRNA expression in their brain tumor tissues may exhibit a more aggressive disease with a worse prognosis. This invention demonstrated that newly diagnosed pediatric DIPG patients with augmented TGFB2 mRNA levels in their primary tumor samples, but not pediatric DIPG patients with augmented transforming growth factor beta 1 (TGFB1) or transforming growth factor beta 3 (TGFB3) mRNA levels, have significantly worse progression-free survival (PFS) as well as overall survival (OS) than other pediatric DIPG patients. High TGFB2 mRNA level was a poor prognostic indicator for DIPG patients but not for DMG patients whose tumors were outside of the pons/brainstem in locations such as the cerebellum and thalamus or pediatric glioblastoma (GBM) patients.

A clinical study was done to determine clinical metadata and RNA sequencing (RNAseq)-based mRNA expression data for 41 pediatric DMG patients (mean age at diagnosis in months=7.02±0.44; median=6; range=2-14) and 116 pediatric GBM patients (mean age at diagnosis in months=60.01±1.24; median=60.4; range=21-89.3) regarding TGFB isoforms TGFB1, TGFB2, and TGFB3 using genomic data obtained via the cBioPortal for Cancer Genomics (https://pedcbioportal.kidsfirstdrc.org/). An interactive web interface was used with full filtering functionality provided by the portal. The data was compiled, harmonized and annotated across multiple data consortiums, such as the open Pediatric Brain Tumor Atlas (PBTA) Project (Project ID=openpbta) and the Pacific Pediatric Neuro-Oncology Consortium Clinical Genomics Atlas (Project ID=pbta_pnoc). This clinical study examined the effect of TGFB2 mRNA expression levels on PFS and OS outcomes. The general treatment strategies are outlined in the clinical trials associated with the treatment of DIPG patients (https://clinicaltrials.gov/ct2/show/NCT02274987) that included standard radiation therapy followed by biomarker-guided specialized therapy with FDA-approved targeted therapeutics drugs, which was guided by gene expression analysis, whole exome-sequencing (WES), and predictive modeling.

The downloaded mRNA expression levels of TGFB1, TGFB2, TGFB3, Transforming Growth Factor Beta Receptor 1 (TGFBR1), Transforming Growth Factor Beta Recep-tor 2 (TGFBR2) and Transforming Growth Factor Beta Receptor 3 (TGFBR3) were reported using the RNAseq V2 values (datafiles for the expression profiles appended with “mRNA_expression_(RNA_Seq_V2_RSEM).txt”) normalized to “transcripts per million” (“TPM”) values calculated using RSEM, which is a software package for estimating gene and isoform expression levels from RNAseq data. The RSEM-based process consists of two main steps: 1. A set of reference transcript sequences are generated for gene level mRNA abundance assessment; 2. A set of RNAseq reads are aligned to these reference transcripts for TPM abundance estimations. This process allows for a direct comparison of mRNA abundance and ranking across samples.

The Kaplan-Meier (KM) method, log-rank chi-square test, and the software packages survival_3.2-13, survminer_0.4.9 and survMisc_0.5.5 operated in the R environment were used to compare the PFS and OS outcomes of patient subsets. Graphical representations of the treatment outcomes were generated using graph drawing packages implemented in the R programming environment: dplyr_1.0.7, ggplot2_3.3.5, and ggthemes_4.2.4. The statistical significance of differences in the outcomes of the compared patient subsets was examined using the log-rank chi-square test and p-values less than 0.05 were deemed significant.

TBFB1, TGFB2, and TGFB3 mRNA expression values for normal pons specimens measured by RNAseq (rna_tissue_hpa.tsv.zip) were downloaded from https://www.proteinatlas.org/about. mRNA expression values were compiled (in TPM) for 29 distinct pons regions of the brain by filtering the “Tissue Group” annotations the accompanying description file (“rna_tissue_hpa_description.tsv.zip”). A two-way analysis of variance (ANOVA) model was used to compare the TGFB1/TGFB2/TGFB3 mRNA expression levels for the normal pons specimens with the TGFB1/TGFB2/TGFB3 mRNA expression levels in brain tumor specimens from 41 DIPG patients. Contrasts were performed between normal pons and DIPG samples for each of the transcripts and p-values were false discovery rate (FDR)-adjusted. Calculations were performed using multcomp_1.4-17 and emmeans_1.7.0 statistical packages in R version 4.1.2 with RStudio front end (RStudio 2021.09.0+351 “Ghost Orchid” Release)). Bar chart graphics were constructed using the ggplot2_3.3.5 R package.

mRNA expression levels of TGFB2 in log 2 TPM were correlated with the mRNA expression levels for 11 transcription factors known to augment TGFB2 expression, namely activating transcription factor 1 (ATF1), activating transcription factor 2 (ATF2), cyclic AMP-responsive element binding protein 1 (CREB1), E1A binding protein P300 (EP300), forkhead box protein 03 (FOXO3), polymerase II subunit A (POLR2A), regulatory factor X1 (RFX1), specificity protein 1 transcription factor (SP1), TATA-box-binding protein (TBP), upstream transcription factor 1 (USF1), and upstream transcription factor 2 (USF2) across 41 DIPG patients. Pairwise correlation coefficients were determined for all transcript combinations and visualized on a heatmap which was color-coded from positive correlations (red=+1) to negative correlations (blue=−1). The clustering algorithm identified the co-regulated sets of genes using the statistical package ggcorrplot_0.1.3 implemented in R. T-test was used to test the null hypothesis that the Pearson correlation coefficient was equal to zero. Significant correlations were identified for p-values less than 0.05 and FDR less than 0.10.

Normalized archived transcriptome profiling datasets acquired from the Gene Expression Omnibus web portal (https://www.ncbi.nlm.nih.gov/geo), including raw CEL files obtained using the Human Genome U133 Plus 2.0 Array platform for DIPG (N=29; GSE26576), normal control samples (N=2; GSE26576), and pediatric GBM patients harboring H3K27M mutations (N=5, GSE34824; N=7, GSE49822) were also utilized as an independent validation dataset to compare the mRNA expression levels of TGFB1, TGFB2, and TGFB3 in normal control samples vs. brain tumor specimens from 41 patients with pediatric DIPG or H3K27M-mutant pediatric GBM. The normalization procedure to determine log 2-transformed mRNA expression levels employed the method of Robust Multi-array Averaging (RMA), as previously described. mRNA expression levels were calculated using Aroma Affymetrix statistical packages (aroma.affymetrix_3.2.0, aroma.core_3.2.2 and aroma.light_3.24.0) run in the RStudio environment (R version 4.1.2, RStudio 2021.09.0 Build 351). Statistical comparisons were performed using an ANOVA statistical model. FDR-adjusted p-values less than 0.05 were deemed significant. mRNA expression levels in DIPG/H3K27M-mutant GBM patients for TGFB1, TGFB2, and TGFB3 were visualized using heatmaps, as described. Expression levels of TGFB2 mRNA (log 2 RMA) were correlated to mRNA a two-way ANOVA model: expression levels for 11 transcription factor genes known to augment TGFB2 expression; ATF1, ATF2, CREB1, EP300, FOXO3, POLR2A, RFX1, SP1, TBP, USF1, and USF2.

### Results.

Tumor Specimens from Pediatric Patients with DIPG Contain Higher Levels of TGFB2 mRNA but Not TGFB1 mRNA or TGFB3 mRNA, Compared to Normal Pons Specimens.

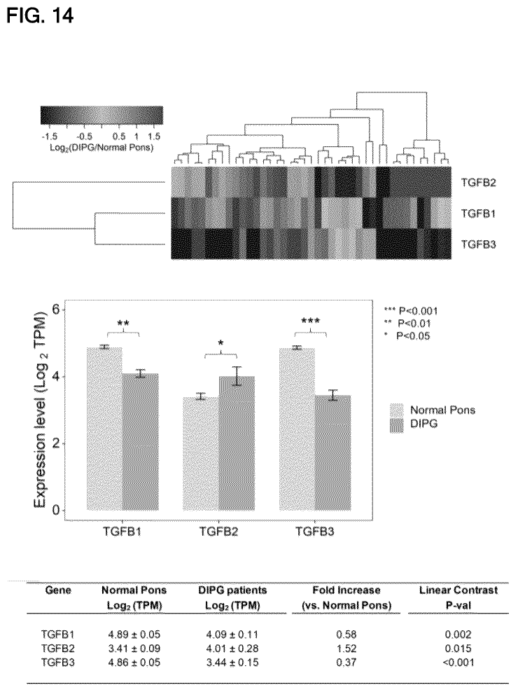

RNAseq-based mRNA levels were compared for TGFB1/2/3 isoforms in 41 primary DIPG samples vs. 29 normal pons specimens (FIG. 14). Notably, the average (mean±SE) TGFB2 mRNA level in primary DIPG samples was 1.5-fold higher than the average TGFB2 mRNA level in normal pons specimens (4±0.3 vs. 3.4±0.1, p=0.015) (FIG. 14). By contrast, both TGFB1 and TGFB3 mRNA levels in DIPG samples were significantly lower (1.7-fold lower TGFB1 mRNA level, p=0.0002 and 2.7-fold lower TGFB3 mRNA level, p<0.001) than the corresponding levels in normal pons samples, as reflected by the blue color in the heat map of the hierarchical cluster (FIG. 14) and significantly lower mean expression values (in log 2 TPM) (FIG. 14).

Selective Overexpression of TGFB2 mRNA in DIPG Tumor Specimens Is Associated with Augmented Expression of Transcription Factors Binding to Multiple TGFB2 Gene Promoter Sites.

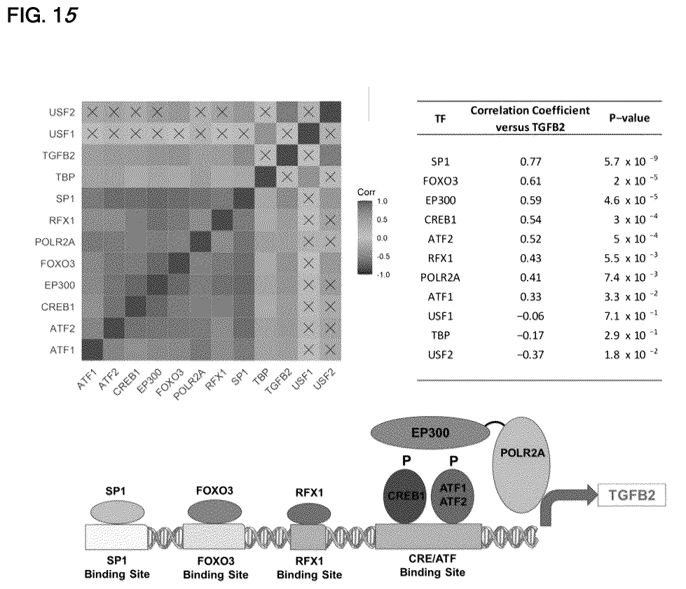

The molecular mechanism was identified for the upregulation of TGFB2 mRNA expression levels in 41 DIPG tumor samples with a focus on transcription factors/DNA binding proteins that are known to augment TGFB2 gene expression. The transcript-level expression of 11 transcription factors were examined with enhancing activity on TGFB2 gene expression, namely ATF1, ATF2, CREB1, EP300, FOXO3, POLR2A, RFX1, SP1, TBP, USF1, and USF2 in relationship to TGFB2 mRNA levels. The mRNA levels from 8 of these 11 transcription factors (viz.: SP1, RFX1, POLR2A, FOXO3, EP300, CREB1, ATF2, and ATF1) exhibited statistically significant positive correlations with TGFB2 mRNA levels (FIG. 15). SP1, FOXO3, and EP300 showed the most significant correlations with TGFB2 mRNA expression levels (p<0.0001).

FIG. 14. Selective upregulation of TGFB2 mRNA expression in DIPG tumor samples. Archived TGFB1/2/3 mRNA expression levels (in log 2-transformed TPM) for primary tumor samples from 41 DIPG patients, including 11 DIPG patients with unknown H3K27M mutational status, 4 H3K27M-mutant DIPG patients, and 26 H3K27M-mutant DMG patients, whose brain tumors were localized to pons/brainstem, obtained from cBioPortal were compared to TGFB1/2/3 mRNA expression levels (log 2 TPM) in normal pons samples from 29 pons regions (downloaded from https://www.proteinatlas.org/about/download). The TGFB2 expression levels for these 29 normal pons regions were determined by averaging the TPM values for 2-8 independent samples/region from 21 subjects. Expression of log 2-transformed TPM values for TGFB1/2/3 mRNA levels in the tumor samples from DIPG patients were mean-centered to the mRNA expression levels in normal pons samples and depicted in the heatmap (A) representing overexpression (red color) or underexpression (blue color). (B) The bar charts illustrate reduced expression levels for TGFB1 and TGFB3 mRNA but amplified expression levels for TGFB2 mRNA, in tumor specimens from DIPG patients (dark grey bars) when compared to normal pons samples (light grey bars).

(C) Statistical significance of differences in TGFB1/2/3 mRNA expression levels (in log 2-transformed TPM values) was assessed using two-way ANOVA with linear contrasts using FDR-adjusted p-values. A statistically significant 1.52-fold increase in TGFB2 mRNA levels (p=0.015) was found along with statistically significant decreases in TGFB1 mRNA (1.72-fold decrease; p=0.002) and TGFB3 (2.7-fold decrease; p<0.001) mRNA levels.

FIG. 15. Correlation matrix of TGFB2 and transcription factors in 41 DIPG patients. Pairwise Pearson correlations were performed between the mRNA levels of TGFB2 and 11 transcription factors using log 2-transformed TPM values obtained from cBioPortal for Cancer Genomics (https://pedcbioportal.kidsfirstdrc.org/). The correlation coefficients were calculated across 41 DIPG patients, depicted on the heatmap (A) ranging from positive correlations (red) to negative correlations (blue) and organized according to similarly expressed genes. The scale for the range of correlations is shown in the color bar, “Corr”. Of the 66 pairwise correlations, 48 were deemed statistically significant (p<0.05 and FDR=0.07; non-significant correlations are indicated with a black cross in the heat map). (B) The mRNA levels for 8 of 11 transcription factors (viz.: SP1, RFX1, POLR2A, FOXO3, EP300/transcription factor co-activator, CREB1, ATF2, and ATF1) exhibited statistically significant positive correlations with TGFB2 mRNA levels. SP1, FOXO3, and EP300 mRNA levels showed the most significant correlations with TGFB2 mRNA expression levels (p<0.0001). (C) Depicted are the transcription factor proteins (oval shapes) bound to binding sites on the TGFB2 gene for these transcription factors (rectangles). Additionally, indicated are the recruitment of the transcription factor co-activator EP300 (E1A Binding Protein P300), that binds to phosphorylated forms of CREB1/ATF1/ATF2 and the bridging to the basal transcription machinery via the RNA Polymerase II Subunit A (POLR2A) to directly stimulate TGFB2 transcription.

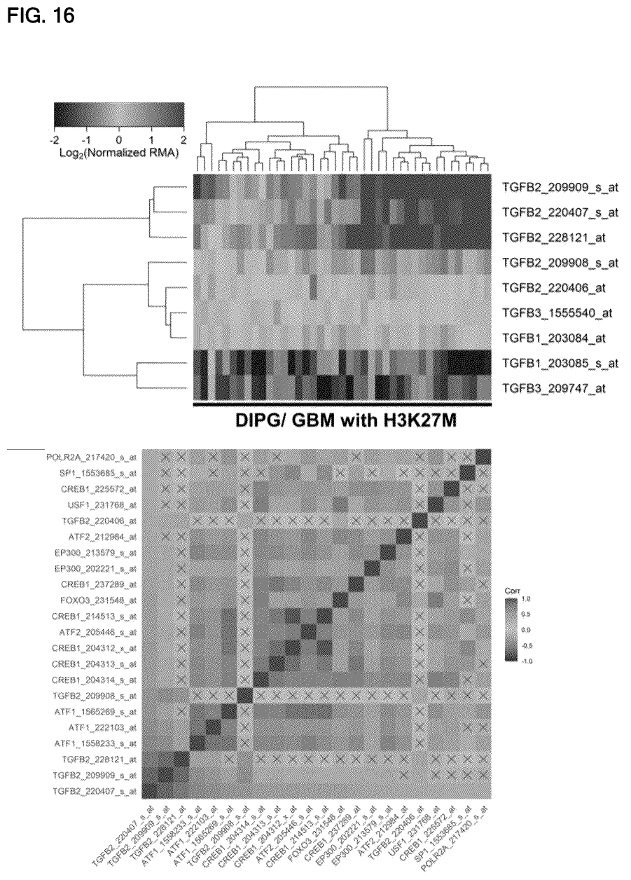

Similar results were obtained in an independent validation dataset of microarray-based mRNA levels for TGFB1, TGFB2, and TGFB3, and their correlated expression with specific transcription factors in tumor specimens from 41 pediatric patients with DIPG (n=29) or H3K27M-mutant GBM (FIG. 16). Significant increases in mRNA expression levels were observed for three TGFB2 probesets: TGFB2_228121_at (2.7-fold increase, p=0.006); TGFB2_209909_s_at (2.4-fold increase, p=0.019); and TGFB2_220407_s_at (2.2-fold increase, p=0.032) (FIG. 16, Panel). A significant decrease was observed in the mRNA expression of one of the TGFB1 probesets, TGFB1_203085_s_at (2.2-fold decrease, p=0.028), and one of the TGFB3 probesets, TGFB3_209747_at (2.3-fold decrease, p=0.026). The probeset TGFB2_220407_s_at exhibited the greatest number of positive correlations with the probesets for the 11 transcription factors and all four other TGFB2 probesets (21 significant positive correlations with p<0.05 (FDR=0.047) (FIG. 16, Panel). The mRNA levels from 8 of the 11 transcription factors (viz.: SP1, USF1, POLR2A, FOXO3, EP300 (2 probesets), CREB1 (6 probesets), ATF2 (2 probesets), and ATF1 (3 probesets)) exhibited statistically significant positive correlations with TGFB2 mRNA levels (FIG. 16, Panel).

FIG. 16. Correlated expression of TGFB2 mRNA and mRNA for specific transcription factors in an independent validation dataset. (A) Differential expression of mRNA levels for TGFB1, TGFB2, and TGFB3 are illustrated using a cluster representation of log 2-transformed fold changes in 41 brain tumor specimens from pediatric patients with DIPG or H3K27M-mutant GBM. The expression levels were mean-centered to normal samples and depicted as log 2-transformed normalized RMA values. The blue to red color in the heatmap indicates under-expression to over-expression, respectively, for the mRNA levels of TGFB1, TGFB2, and TGFB3. Co-regulated probesets are organized and depicted by dendrograms for both probesets (rows) and patients (columns). Significant increases in mRNA expressions were observed for three TGFB2 probesets: TGFB2_228121_at (2.7-fold increase, p=0.006); TGFB2_209909_s_at (2.4-fold increase, p=0.019); and TGFB2_220407_s_at (2.2-fold increase, p=0.032). A significant decrease was observed in the mRNA expression of one of the TGFB1 probesets, TGFB1_203085_s_at (2.2-fold decrease, p=0.028), and one of the TGFB3 probesets, TGFB3_209747_at (2.3-fold decrease, p=0.026). (B) A total of 1640 correlations were performed for 41 probe-sets representing 11 transcription factors and TGFB2 mRNA. For 660 correlations, the p-values were less than 0.05 (FDR=0.12), and 324 correlations exhibited p-values less than 0.001 (FDR=0.005). The probeset TGFB2_220407_s_at exhibited the greatest number of positive correlations with the probesets for the 11 transcription factors and all four other TGFB2 probesets. There were 21 significant positive correlations with p<0.05 (FDR=0.047). Color-coded Pearson correlation coefficients for the most significant positive correlations with TGFB2_220407_s_at are depicted on the heatmap ranging from positive correlations (red) to negative correlations (blue) organized according to similarly expressed mRNA levels. The scale for the range of correlations is shown in the color bar, “Corr”. Non-significant correlations are indicated with a black cross in the heat map. The mRNA levels from 8 of the 11 transcription factors (viz.: SP1, USF1, POLR2A, FOXO3, EP300 (2 probesets), CREB1 (6 probesets), ATF2 (2 probesets), and ATF1 (3 probesets)) exhibited statistically significant positive correlations with TGFB2 mRNA levels.

Amplified Expression of TGFB2 mRNA but Not TGFB1 or TGFB3 mRNA, Is Associated with Shorter OS and PFS in DIPG Patients.

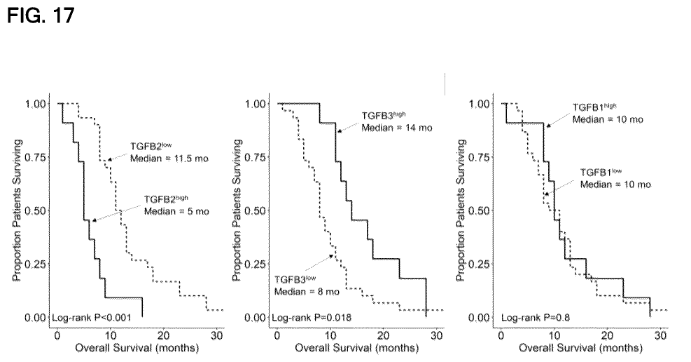

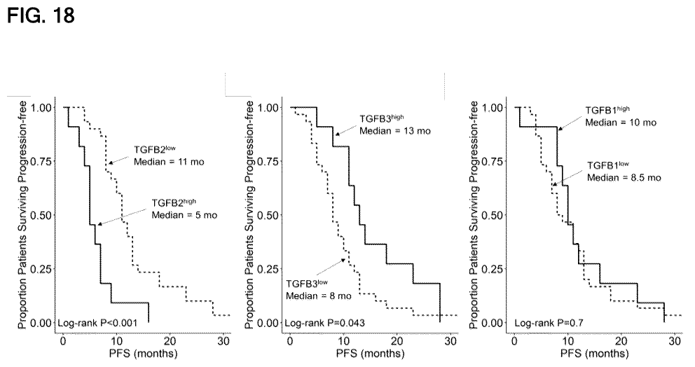

Survival outcomes of DIPG patients were compared with TGFB2 mRNA expression levels greater than or equal to the upper quartile (TGFB2high) to the treatment outcomes of the remaining DIPG patients (TGFB2low). The mean expression level for TGFB2 in the 11-patient TGFB2high subset was 6.2±0.2 (median, range=5.8, 5.2-7.6). By comparison, the mean TGFB2 mRNA expression level for the 30-patient TGFB2low subset was 3.2±0.2 (median, range=3.5, −0.4-5.1). TGFB2high DIPG patients exhibited a significantly worse OS outcome than TGFB2low DIPG patients (median OS for TGFB2high subset: 5 months, 95% CI: 5-NA months, 11 events, N=11); median OS for TGFB2low subset: 11.5 months, 95% CI: 10-14 months, 30 events, N=30; log-rank chi-square=16.2, p=5.6×10-5) (FIG. 17). The PFS outcome was also significantly worse for the TGFB2high subset (median PFS for TGFB2high subset: 5 (95% CI: 5-NA) months, 11 events, N=11) vs. median PFS for TGFB2low subset=11 months, 95% CI: 10-13 months, 30 events, N=30 (log-rank chi-square=14.7, p=1.3×10-4) (FIG. 18). In contrast to the poor prognostic impact of high TGFB2 mRNA expression, high TGFB3 mRNA expression was associated with significantly better OS and PFS outcomes, and high TGFB1 mRNA expression had no statistically significant effect on OS or PFS outcomes (FIGS. 17 and 18).

The OS outcome data for the 30 H3K27M-mutant DIPG patients was separately evaluated excluding the 11 DIPG patients with an unknown H3K27M mutational status. TGFB2high patients within the 30-patient subset had a shorter time to death than the remaining patients, similar to the TGFB2high patients in the 41-patient full analysis set, which included 11 DIPG patients with an unknown H3K27M mutational status (not shown). However, the difference did not reach statistical significance in the smaller subset, which is likely due to the reduction of power to detect a statistical difference arising from the broader distribution of OS outcomes and smaller sample size (not shown). Notably, as with the full analysis set of 41 patients (not shown), TGFB2high patients in the 30-patient subset were characterized by early failures and significantly worse survival outcomes within 10 months (not shown). The median survival for TGFB2high patients within the 30-patient H3K27M-mutant subset was 6.5 months (95% CI=5-NA months, 6 events, N=8) which was significantly shorter than the median survival for the remaining patients (median >10 months, 7 events, N=22; log-rank chi-square=5.5, p-value=0.019) (not shown).

FIG. 17. Amplified expression of TGFB2 is associated with shorter OS in DIPG patients. Archived survival outcome data for 41 DMG patients, including 11 DIPG patients with unknown H3K27M mutational status, 4 H3K27M-mutant DIPG patients and 26 H3K27M-mutant DMG patients, whose brain tumors were localized to pons/brainstem, obtained from cBioPortal for Cancer Genomics (https://pedcbioportal.kidsfirstdrc.org/) was combined with RNAseq-based mRNA expression data for of TGFB2 (A), TGFB3 (B), and TGFB1 (C) to assess the potential impact of the respective TGFB isoform levels on OS. Log 2-transformed, TPM-normalized RNAseq values were rank-ordered according to expression level for each TGFB isoform. Patients whose expression levels for a given TGFB isoform was greater than or equal to the upper quartile (solid line) were compared to the remaining patients (dashed line). See the legend of FIG. 14 for the TGFB isoform levels in the analyzed patient subsets. (A) The mean expression level for TGFB2 in the 11-patient TGFB2high subset was 6.19±0.24 (median, range=5.81, 5.23-7.63). By comparison, the mean TGFB2 expression level for the 30-patient TGFB2low subset was 3.22±0.23 (median, range=3.48, −0.42-5.07). Patients with high TGFB2 mRNA expression (TGFB2high) exhibited a significantly worse OS outcome than rest of the patients (TGFB2low) (median OS for TGFB2high subset: 5 months, 95% CI: 5-NA months, 11 events, N=11); median OS for TGFB2low subset: 11.5 months, 95% CI: 10-14 months, 30 events, N=30; log-rank chi-square=16.2, p=5.6×10-5). (B) The mean expression level for TGFB3 in the 11-patient TGFB3high subset was 4.7±0.2 (median, range=4.6, 4-5.6). By comparison, the mean TGFB3 expression level for the 30-patient TGFB3low subset was 3±0.1 (median, range=3.1, 1.5-4). Patients with high TGFB3 expression (TGFB3high) (median OS: 14 months, 95% CI: 12-NA months, 11 events, N=11) exhibited a significantly better OS outcome than TGFB3low patients (median OS: 8 months, 95% CI: 7-11 months, 30 events, N=30; log-rank chi-square=5.6, p=0.018). (C) The mean expression level for TGFB1 mRNA in the 11-patient TGFB1high subset was 4.8±0.1 (median, range=4.8, 4.5-5.4). By comparison, the mean TGFB1 expression level for the 30-patient TGFB1low subset was 3.8±0.1 (median, range=3.9, 1-4.5). Patients with high TGFB1 expression (TGFB1high) (median OS=10 months, 95% CI: 9-NA months, 11 events, N=11) exhibited similar OS outcomes when compared to the rest of the patients (TGFB1low) (median OS=10 months, 95% CI: 8-13 months, 30 events, N=30; log-rank chi-square=0.1, p=0.8.

FIG. 18. Amplified expression of TGFB2 is associated with shorter PFS in DIPG and DMG patients. Archived clinical outcome data for 41 DMG patients, including 11 DIPG patients with an unknown H3K27M mutational status, 4 H3K27M-mutant DIPG patients and 26 H3K27M-mutant DMG patients, whose brain tumors were localized to brainstem/pons, obtained from cBioPortal for Cancer Genomics (https://pedcbioportal.kidsfirstdrc.org/) was combined with RNAseq-based mRNA expression data for TGFB2 (A), TGFB3 (B), and TGFB1 (C) to assess the potential impact of the respective TGFB isoform levels on PFS. For patients with missing information regarding progression of disease, death was used as the first event when evaluating the PFS outcome. Log 2-transformed, TPM-normalized RNAseq values were rank-ordered according to expression level for each TGFB isoform. Patients whose expression levels for a given TGFB isoform was greater than or equal to the upper quartile (solid line) were compared to the remaining patients (dashed line). (A) TGFB2high patients with exhibited a significantly worse PFS outcome than rest of the patients (median PFS for TGFB2high subset: 5 (95% CI: 5-NA) months, 11 events, N=11); median PFS for TGFB2low subset=11 months, 95% CI: 10-13 months, 30 events, N=30; log-rank chi-square=14.7, p=1.3×10-4). (B) TGFB3high patients (median PFS=13 months, 95% CI: 11-NA months, 11 events, N=11) exhibited a significantly better PFS outcome than TGFB3low patients (median PFS=8 months, 95% CI: 7-11 months, 30 events, N=30; log-rank chi-square=4.1, p=0.043). (C) TGFB1high patients (median PFS=10 months, 95% CI: 9-NA months, 11 events, N=11) and TGFB1low patients exhibited similar OS outcomes (median PFS=8.5 months, 95% CI: 7-13 months, 30 events, N=30; log-rank chi-square=0.2, p=0.7).

TGFB2 Expression Level Does Not Affect OS or PFS in Pediatric DMG Patients Whose Tumor Is Not Located in Pons/Brainstem.

Survival outcomes were compared of non-DIPG DMG patients whose tumor location was outside the pons/brainstem with TGFB2 mRNA expression levels greater than or equal to the upper quartile (TGFB2high) to the treatment outcomes of the remaining non-DIPG DMG patients (TGFB2low). TGFB2high patients (median OS=12.5 months, 95% CI: 9-NA months, 10 events) exhibited a similar OS outcome when compared to TGFB2low patients (median OS=11 months, 95% CI: 2-NA months, 8 events; log-rank chi-square=0.2, p=0.6) (not shown). TGFB2high and TGFB2low non-DIPG DMG patients also had very similar PFS outcomes (not shown). Similarly, no statistically significant differences in OS or PFS outcomes were found in TGFB1high vs. TGFB1low or TGFB3high vs. TGFB3low non-DIPG DMG subset comparisons (not shown). Since the activation of TGFB signaling pathways requires binding of TGFB to the TGF-β receptor II (TGF-βRII), which recruits TGF-βRI into a heterotetrameric complex, it was determined if the observed lack of a prognostic effect of TGFB2high status could be due to decreased TGFB2 receptor expression levels. None of the TGFB2 receptors exhibited a lower expression level in tumor samples from non-DIPG DMG patients (N=19) compared to the tumor samples from DIPG patients (N=41) to explain the observed lack of prognostic significance of higher TGFB2 mRNA levels in this patient group (not shown). The DIPG patients were on average younger (mean age at diagnosis in months=7.0±0.4; median=6; range=2-14) than the non-DIPG DMG patients (mean age at diagnosis in months=10.5±0.7; median=11; range=5-17) (p=0.0003).

TGFB2 mRNA Expression Level Does Not Affect OS or PFS in Pediatric GBM Patients.

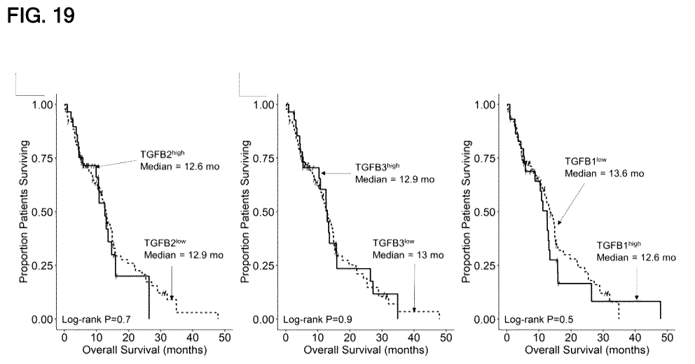

survival outcomes were compared of pediatric GBM patients whose TGFB2 mRNA expression levels were greater than or equal to the upper quartile (TGFB2high) to the treatment outcomes of the remaining pediatric GBM patients (TGFB2low). The mean expression level for TGFB2 in the 29-patient TGFB2high pediatric GBM subset was 12.7±0.1 (median, range=12.6, 12-13.7). By comparison, the mean expression level for TGFB2 in the 87-patient TBFB2low pediatric GBM subset was 10.4±0.1 (median, range=10.5, 8.2-12). Patients in the TGFB2high (N=29) and TGFB2low (N=87) subsets exhibited very similar OS outcomes (median for TGFB2high: 12.6 (95% CI: 9.9-NA) months, 17 events; median for TGFB2low: 12.9 (95% CI: 11.2-15) months, 65 events; log-rank chi-square=0.2, p=0.7) (FIG. 19). TGFB2high and TGFB2low pediatric GBM patients also had very similar PFS outcomes (not shown). Similarly, no differences in OS or PFS outcomes were found in TGFB1high vs. TGFB1low or TGFB3high vs. TGFB3low pediatric GBM subset comparisons (FIG. 18). None of the TGFB2 receptors exhibited a lower expression level in tumor samples from GBM patients vs. DIPG/DMP patients to explain the observed lack of prognostic significance of higher TGFB2 mRNA levels in this patient population. To the contrary, GBM patients exhibited significantly higher levels of all three receptors compared to DIPG patients (not shown). The TGFB2 mRNA levels in GBM patients were higher than those of DIPG patients; the mean expression level for TGFB2 mRNA even in the 11-patient TGFB2high DIPG subset was 6.19±0.24 (median, range=5.81, 5.23-7.63), which is lower than the TGFB2 mRNA levels of TGFB2low GBM patients (10.4±0.1; median, range=10.5, 8.2-12). The absence of a truly TGFB2low subset in GBM patients might have precluded an accurate evaluation of the prognostic effect of high TGFB2 mRNA levels. The DIPG patients were on average younger (mean age at diagnosis in months=7.0±0.4; median=6; range=2-14) than the GBM patients (mean age at diagnosis in months=60.0±1.2; median=60.4; range=21-89.3) (p<0.0001).

FIG. 19. TGFB2 mRNA expression level does not affect OS in pediatric GBM patients. Archived OS data for 116 GBM patients obtained from cBioPortal for Cancer Genomics (https://pedcbioportal.kidsfirstdrc.org/) was combined with RNAseq-based mRNA expression data for TGFB2 (A), TGFB3 (B), and TGFB1 (C) to assess the potential impact of the respective TGFB isoform levels on OS. Log 2-transformed, TPM-normalized RNAseq values were rank-ordered according to expression level for each TGFB isoform. Patients whose expression levels for a given TGFB isoform was greater than or equal to the upper quartile (solid line) were compared to the remaining patients (dashed line). See the legend of FIG. 16 for the TGFB isoform levels in the analyzed patient subsets. (A) The mean expression level for TGFB2 in the 29-patient TGFB2high subset was 12.67±0.1 (median, range=12.63, 11.97-13.7). By comparison, the mean expression level for TGFB2 in the 87-patient TBFB2low subset was 10.37±0.1 (median, range=10.49, 8.17-11.94). Patients in the TGFB2high (N=29) and TGFB2low (N=87) subsets exhibited very similar OS outcomes (median for TGFB2high: 12.6 (95% CI: 9.9-NA) months, 17 events; median for TGFB2low: 12.9 (95% CI: 11.24-14.95) months, 65 events; log-rank chi-square=0.2, p=0.7). (B) The mean expression level for TGFB3 in the 29-patient TGFB3high subset was 11.39±0.1 (median, range=11.25, 10.63-12.88). By comparison, the mean expression level for TGFB3 in the 87-patient TBFB3low subset was 9.4±0.08 (median, range=9.56, 7.14-10.58). Patients in the TGFB3high and TGFB3low subsets exhibited very similar OS outcomes (median for TGFB3high: 12.9 (95% CI: 10.41-26.38) months, 20 events); median for TGFB3low: 13 (95% CI: 11-14.95) months, 62 events; log-rank chi-square=0, p=0.9). (C) The mean expression level for TGFB1 in the 29-patient TGFB1high subset was 12.08±0.08 (median, range)=11.92, 11.58-13.13). By comparison, the mean expression level for TGFB1 in the 87-patient TBFB1low subset was 10.53±0.09 (median, range=10.71, 7.5-11.56). TGFB1high patients (median: 12.6 (95% CI: 8.8-15.8) months, 22 events; N=29) and TGFB1low patients (median: 13.6 (95% CI: 11.7-15.4) months, 60 events) exhibited similar OS outcomes (log-rank chi-square=0.5, p=0.5.

In this clinical study, the prognostic significance of TGFB2high status was examined in newly diagnosed pediatric DIPG patients. Notably, TGFB2high (but not TGFB1high or TGFB3high) patients had significantly worse survival outcomes with substantially shorter OS times. RNAseq-based high TGFB2 mRNA level was an indicator of poor prognosis for DIPG patients, but not for pediatric GBM patients or pediatric DMG patients with tumor locations outside of the pons/brainstem. The lack of an adverse prognostic effect for higher TGFB2 mRNA expression levels in the latter patient populations was not due to lower expression levels of TGFB2 receptors. It is noteworthy that the TGFB2 mRNA levels in pediatric GBM patients were markedly higher than those of DIPG patients. The absence of a truly TGFB2low subset in GBM patients might have precluded an accurate evaluation of the favorable prognostic effect of low TGFB2 mRNA levels.

This study provides significant understanding of the clinical significance of high TGFB2 expression in pediatric high-grade gliomas. The TGFB2-promoted invasive growth of DIPG cells, their TGFB2-associated radiation resistance, and possibly TGFB2-mediated restrictions of the cellular anti-glioma immunity within the TME contributed to the observed adverse impact of high TGFB2 levels on the survival outcomes of DIPG patients.

In a clinical study using a microarray-based gene expression platform, it was found that TGFB2 transcript, but not TGFB1 or TGFB3 levels were selectively amplified in primary tumor samples from 29 pediatric DIPG patients compared to normal samples and primary tumor samples from grade low-grade glioma patients. In this study, the RNAseq-based TGFB2 mRNA levels were compared in primary DIPG tumor samples and normal control pons samples. Notably, tumor samples from DIPG patients expressed significantly higher levels of TGFB2 mRNA than control pons samples, but the TGFB1, as well as TGFB3 mRNA levels in DIPG samples were significantly lower than in the control pons samples. These results generated using the RNAseq-based mRNA data confirm and significantly expand our previous results obtained using the microarray platform. The augmented expression of the TGFB2 gene in DIPG samples might be due to the enhanced expression of transcription factors that upregulate TGFB2 mRNA expression. This hypothesis was supported by the finding of a strong positive correlation (p<0.0001) between the mRNA levels of several such transcription factors, including SP1, FOXO3, and EP300 and TGFB2 mRNA levels in DIPG samples. Similar results were obtained in an independent validation dataset of microarray-based mRNA levels for TGFB1, TGFB2, and TGFB3, and their correlated expression with specific transcription factors in tumor specimens from 41 pediatric patients with DIPG (N=29) or H3K27M-mutant GBM.

There are three isoforms of TGFB within the TGFB superfamily of genes, namely TGFB1, TGFB2, and TGFB3. Despite a >70% sequence homology, the structure and biological functions of the TGFB isoform TGFB3 differs from those of TGFB1 and TGFB2. Notably, TGFB3-knockout mice are uniquely different from TGFB1-knockout or TGFB2-knockout mice. Furthermore, TGFB3 exerts a cancer-preventive effect in non-clinical models of tumor development as well as human subjects. Additionally, high TGFB3 expression has a favorable prognostic effect in breast cancer, ovarian cancer, and colon cancer. However, TGFB3 expression is correlated with a poor prognosis in osteosarcoma. In this study, high TGFB3 expression was identified as a favorable prognostic indicator which contrasts with its reported adverse prognostic role in breast cancer. Our observation expands our current knowledge and provides new insights regarding the multifunctional role of TGFB3.

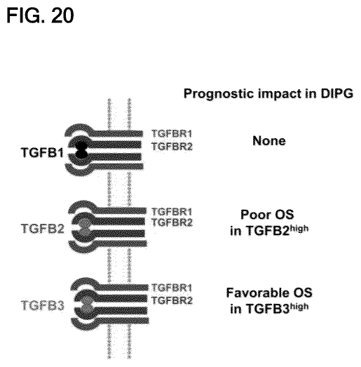

Based on the results presented herein, amplified TGFB2 mRNA expression is associated with poor prognosis and OS in DIPG (FIG. 20).

FIG. 20. TGFB2 mRNA expression is selectively amplified and associated with poor OS in pediatric DIPG. mRNA levels for TGFB2 were selectively amplified in primary brain tumor specimens from DIPG patients, whereas the mRNA levels for TGFB1 and TGFB3 were not. High levels of TGFB2 mRNA expression was associated with poor OS. By contrast, high TGFB1 mRNA expression had no prognostic value and high TGFB3 mRNA expression was associated with favorable OS.

OT-101, a TGFB2-targeting S-ODN, exhibited single-agent clinical activity in adult patients with recurrent or refractory glioblastoma and anaplastic astrocytoma when administered intratumorally via CED. Of the 77 high-grade glioma patients in the efficacy population, 26 had a favorable response, including 19 patients who had a CR or PR and 7 patients who had a stable disease with a longer than 6-month duration. The median PFS for this subset was 1109 days and their OS was 1280 days. The observed poor prognosis of newly diagnosed TGFB2high DIPG patients, as reported here, supports the notion that further exploration of the clinical potential of TGFB2-targeting RNAi therapeutics in TGFB2high DIPG patients is warranted. CED catheters have been used in DIPG patients for intratumoral delivery of therapeutic agents in an attempt to bypass the blood-brain barrier and achieve higher intratumor concentrations while mitigating the risk of systemic toxicity. However, results are limited because of the requirement for months-long intratumoral administration of drug for achieving objective responses in high-grade adult glioma patients, combined with the practical challenges of prolonged use of implanted CED catheters or repeated replacement surgeries for CED catheters at this difficult anatomic location.

This invention provides new delivery methods and/or formulation strategies for TGFB2-targeting therapeutics to be effective in DIPG patients.

### Example 7

### OT-101 Single Agent Activity in Recurrent/Refractory High-Grade Glioma Patients.

Phase 2 clinical data showing remarkable single agent activity of OT-101 in recurrent/refractory high-grade glioma patients with more than a third of patients (26 of 77) receiving the intended 4-11 cycles of therapy achieving durable complete responses, partial responses, or prolonged stable disease and a median OS of 1280 days (95% CI: 1116->1743 days).

The median PFS for these 77 patients was significantly better than the PFS for the 12 patients treated with 1-3 cycles of OT-101 (86 days vs. 32 days, Log-rank P-value<0.0001). Likewise, the median OS of the 77 patients who were treated with 4-11 cycles of OT-101 was significantly better than the median OS for the 12 patients who were treated with 1-3 cycles (432 days vs. 128 days, Log-rank P-value<0.0001).

19 achieved durable objective responses (CR: 3, PR: 16). The median time for 90% reduction of the baseline tumor volume was 11.7 months (Range: 4.9-57.7 months). The mean log reduction of the tumor volume in these 19 patients was 2.2±0.4 (Median=1.4: Range: 0.4-4.5) logs.

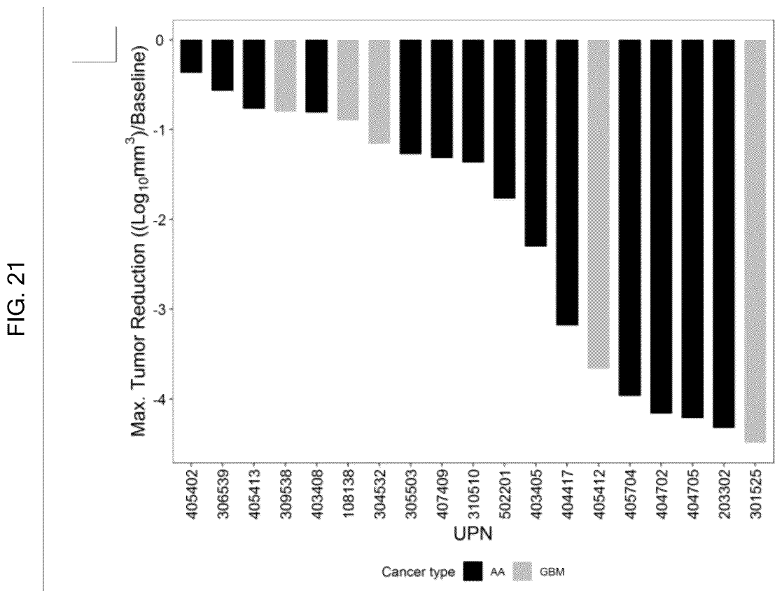

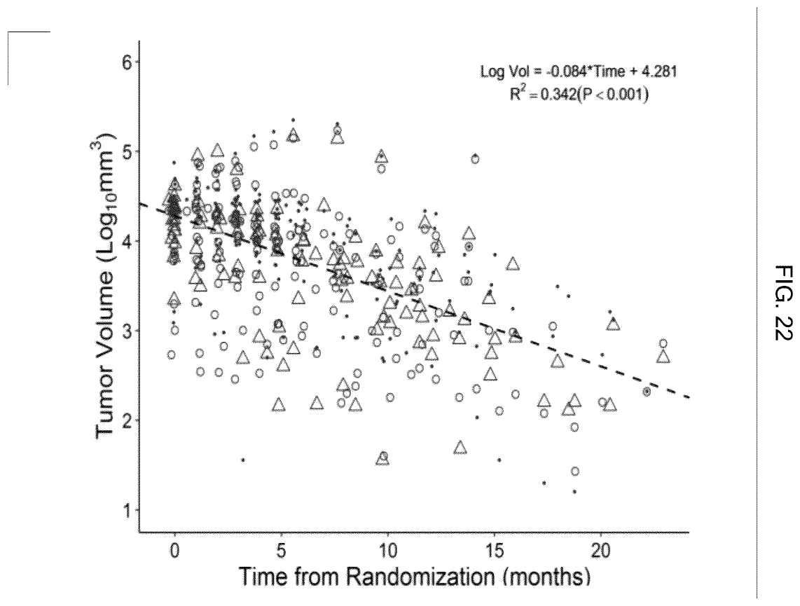

FIG. 21. Imaging Responses in R/R High-Grade Glioma Patients Treated with OT-101 Monotherapy Who Achieved a CR or PR. FIG. 21: A waterfall plot depicting the maximum log 10 reduction values for the tumor volumes.

FIG. 22. Imaging Responses in R/R High-Grade Glioma Patients Treated with OT-101 Monotherapy Who Achieved a CR or PR. FIG. 22: A semi-log plot of the combined 3-D tumor volume reduction curve for the 19 patients.

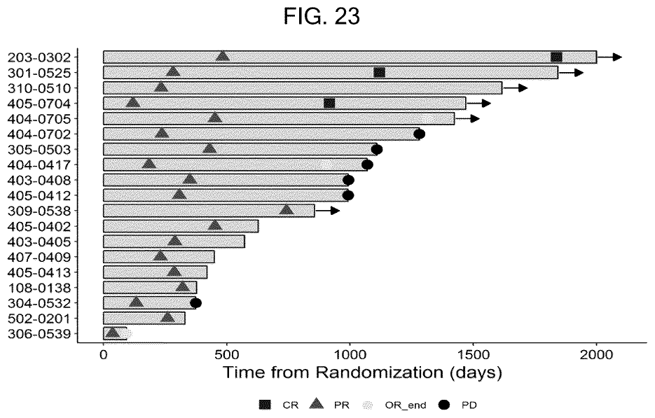

FIG. 23 shows a swimmer plot for onset and duration of objective response. The onset and duration of CR/PR, end of OR and onset of PD are indicated with specific signals.

OT-101 induces durable CR and PR in R/R GBM as well as AA patients. 19 patients had objective responses. 16 had a partial response with an onset at 307±159 days (mean±SE). The median time to onset of PR was 287 days (Range: 37-742). 3 patients had a PR first which deepened to a CR at 917, 1120 and 1838 days, respectively. Six of these 16 patients developed a PD at 970±126.7 days (mean±SE) (Median=1032 days, Range: 374-1281 days).

### Example 8

Clinical Potential of Targeting Transforming Growth Factor 32 with OT-101 for Post-Radiation Consolidation in Diffuse Intrinsic Pontine Glioma.

Diffuse intrinsic pontine glioma (DIPG) in children has a dismal prognosis with a median overall survival (OS) of 10 months and a 2-year overall survival rate of <10% after standard radiation therapy. Chemotherapy does not offer clinically meaningful benefits. Therefore, there is an urgent need for therapeutic innovations for treatment of pediatric DIPG.

High-grade glioma cells, including pediatric glioblastoma and DIPG cells have been shown to produce transforming growth factor beta 2 (TGF-β2) which has been implicated both as promoter of glioma cells and as a key contributor to the T-cell hyporesponsiveness of the tumor microenvironment (TME) towards glioma cells.

OT-101 is a first-in-class RNA therapeutic designed to abrogate the immunosuppressive and tumor promoting actions of TGF-β2. At low micromolar concentrations, OT-101 reduces the TGF-β2 secretion by human glioma cells, blocks their proliferation as well as migration, and restores the anti-glioma cytolytic function of patient-derived T-cells.

The intrathecal/intraventricular administration of antineoplastic drugs directly in the CSF allows to bypass the selective filter of BBB, achieving significant concentrations of the antineoplastic agents in CSF, while reducing the likelihood of systemic toxicity. Informed by favorable safety pharmacology studies of intrathecally delivered OT-101 in rabbits and primates and encouraged by its single agent activity in adult patients with HGG, a multi-center, two-part, randomized Phase 1-2 study of OT-101 in pediatric patients with DIPG was performed. Multiple doses of OT-101 were administered after completion of radiation therapy as intrathecal (IT)/intraventricular bolus injections. The study is designed to determine: 1) the maximum tolerated dose (MTD) or recommended Phase 2 dose (RP2D) of OT-101, and 2) its efficacy in children with DIPG.

Antisense oligodeoxynucleotides are short strings of DNA that are designed to downregulate gene expression by interfering with the translation of a specific encoded protein at the mRNA level. Several RNA therapeutics, including anti-sense oligonucleotides have been evaluated in clinical trials and some approved. OT-101 is a synthetic 18-mer phosphorothioate oligodeoxynucleotide (S-ODN) in which a non-bridging oxygen of each phosphate moiety is substituted by a sulfur atom. OT-101 was designed to be complementary to a specific sequence of human TGF-β2 mRNA following expression of the gene. It is a first-in-class RNA therapeutic designed to abrogate the immunosuppressive actions of TGF-β2 and reduce the level of TGF-β2 in malignant gliomas, and thereby delay the progression of disease.

Functional in vitro assays showed that:

OT-101 exhibits an efficient time-dependent uptake into human tumor cells in the presence as well as in the absence of the carrier liposome Lipofectin®.

OT-101 reduces the TGF-β2 secretion by human tumor cells without the use of any carrier.

At the clinically used OT-101 concentrations up to 80 μM over 7 days in A 172 human high-grade glioma cells, 10 μM is the most effective concentration for inhibition of the TGF-β2 production.

OT-101 reduces proliferation of human tumor cells while at the same time stimulating PBMC proliferation. OT-101 does not affect viability of human PBMC.

OT-101 restores immune function of human PBMC derived from high grade glioma patients demonstrated by immune cell-mediated cytotoxicity assay.

OT-101 inhibits human tumor cell migration.

OT-101 has been subjected to a series of nonclinical safety and pharmacology studies. The salient features of the conclusions from these studies were as follows:

Prolonged local administration of OT-101/AP 12009 may result in local tissue inflammation There was mild to moderate local toxicity observed in animals after infusion of a concentration of 500 μM without any macroscopic changes.

When administered to 3 kg male or female rabbits as a bolus IT injection at a 0.12 mg/kg dose level (500 μM solution; 0.1 mL) with an estimated CSF concentration of 4.16 μM, OT-101 did not cause any clinical toxicity or drug-related macroscopic/microscopic changes consistent with sub-clinical toxicity.

When administered to 6-7 kg cynomolgus monkeys as a bolus IT injection at a 0.05 mg/kg dose level (500 μM solution; 0.1 mL) with an estimated CSF concentration of 0.46 μM, OT-101 did not cause any clinical toxicity or drug-related macroscopic/microscopic changes consistent with sub-clinical toxicity.

When given to rats via intraventricular administration, radiolabeled OT-101 (0.18 mg/kg) was detected within 1 hour after administration not only in the CSF but also in the cerebrum, cerebellum and pineal body. The CSF and brain tissue half-lives were <24 hours with <15% residual OT-101 remaining at 72 hours.

### Example 9

### Nonclinical In Vitro Studies of OT-101/AP 12009.

Functional in vitro assays showed that:

OT-101 exhibits an efficient time-dependent uptake into human tumor cells in the presence as well as in the absence of the carrier liposome Lipofectin®.

OT-101 reduces the TGF-β2 secretion by human tumor cells without the use of any carrier.

At the clinically used OT-101 concentrations up to 80 μM over 7 days in A 172 human high-grade glioma cells, 10 μM is the most effective concentration for inhibition of the TGF-β2 production.

OT-101 reduces proliferation of human tumor cells while at the same time stimulating PBMC proliferation. OT-101 does not affect viability of human PBMCs.

OT-101 restores immune function of human PBMC derived from high grade glioma patients demonstrated by immune cell-mediated cytotoxicity assay.

OT-101 inhibits human tumor cell migration.

Fluorescent signal increased up to 48 h in human A-172 glioblastoma cells both incubated with FITC-OT-101 with or without Lipofectin®. Uptake of FITC-OT-101 was observed already after 3 h incubation time with and without Lipofectin®. After 48 h the fluorescent signal was detectable in almost all cells and was comparable in intensity in cell preparations incubated with or without Lipofectin®.

### Example 10

### Effects of OT-101 on TGF-β2 Synthesis and Secretion by a Human GBM Cell Line.

Cells were incubated with the indicated different concentrations of OT-101/AP 12009 (1 μM to 80 μM) for 7 days. Secreted TGF-β2 was measured in cell supernatants by ELISA. Results represent median, minimum, and maximum values from 3 independent experiments.

The ability of OT-101 to reduce TGF-β2 secretion by primary human glioma cells was determined by measuring the TGF-β2 concentration in cell culture supernatants using an enzyme-linked immunosorbent assay (ELISA). Glioma cells from 10 high-grade glioma patients were cultured for 72 h (HTZ-209. −220, −243, −262, −349, −361, −378, −381) or 96 h (A-172) in the presence and absence of OT-101 (5 or 10 μM). In 8 of the 10 glioma cell cultures, the TGF-β2 secretion was reduced by up to 87%.

OT-101-mediated inhibition of human high-grade glioma cell proliferation.

Two human HGG cell cultures (HTZ-243 and HTZ-349, representing WHO grade III and IV) were incubated with OT-101 (1 μM to 10 μM). The results showed a concentration- and time-dependent reduction of cell numbers within 6 days, as shown in Table 20.

TABLE 20
Effect of OT-101 on Human High-Grade Glioma Cell Proliferation

| | | Cell number [% of cells | | |
|---|---|---|---|---|
| Human glioma | OT-101/AP 12009 | plated] on Day | | |
| cell line | concentration [μM] | 0 | 3 | 6 |
| HTZ-243 | Untreated | 100 | 105 | 110 |
| | 1 | 100 | 98 | 85 |
| | 5 | 100 | 85 | 55 |
| | 10 | 100 | 88 | 48 |
| HTZ-349 | Untreated | 100 | 109 | 122 |
| | 1 | 100 | 96 | 84 |
| | 5 | 100 | 93 | 47 |
| | 10 | 100 | 94 | 50 |

Two human glioma cell cultures (HTZ-243 and HTZ-349) were treated with OT-101 (1, 5 or 10 μM). Cell number (in % of cell number at start of the experiment) was measured with a hemacytometer. Data show the means of duplicate assessment.

### Example 11

### Administration OT-101 (AP 12009).

This study assessed two doses of AP 12009 in parallel treatment groups. AP 12009, at a concentration of 10 μM or 80 μM was administered intratumorally using continuous convection enhanced delivery at a flow rate of 4 μL/min over a 7-day period every second week. The regimen of AP 12009 was based on the clinical Phase I/II data from three studies:

Concentration: AP 12009 proved to be safe up to the maximum evaluated concentration of 80 μM. Flow rate: some results from Study G003 indicated that the infused volume might constitute an important safety aspect, especially in patients with large sized tumors, who were at high risk to develop brain edema rather quickly because the brain's capacity to compensate increasing intracranial pressure was likely to be exhausted. While administration at a flow rate of 8 μL/min over four days was assessed as safe, it could not be excluded that AEs of higher severity appeared more often at 8 μL/min over seven days. Balancing between safety and efficacy considerations, it was decided that the infusion period was to be kept at 7 days, but the flow rate reduced to 4 μl/min (total volume applied in each treatment cycle: 40.32 mL). Thus, a longer drug exposure could be achieved, but the infused volume was reduced to half compared with the infusion speed of 8 μL/min over four days. The flow rate for the isotonic saline infusion during the 7-day interval was set to 1 μL/min to reduce the risk to develop symptoms of increased intracranial pressure.

Due to the significant dead space volume of the application system administration of relevant concentrations of AP 12009 into the brain was delayed at start of each treatment cycle. It had been determined that after 10 hours 95% of the nominal concentration of AP 12009 was reached. This delay at the beginning of each AP 12009 infusion had to be compensated to allow the complete administration of the nominal dose of AP 12009 within the defined infusion period. Therefore, after switching to infusion of isotonic saline the flow-rate was 4 μL/min for equivalent 10 hours at the beginning of the interval period in order to rinse the remaining AP 12009 out of the dead space. Then the flow-rate was reduced to 1 μL/min.

### Example 12

### Effects of OT-101 and Chemotherapy in Glioma.

Non-inferiority analysis was performed in terms of overall survival (OS) of patients receiving either OT-101 or standard chemotherapy (TMZ, PCV, or BCNU). A total of 156 subjects, 101 subjects in the OT-101 test group (G004: 89 subjects, G005: 12 subjects) and 55 subjects in the standard chemotherapy control group (G004: 45 subjects, G005: 10 subjects), were assessed.

Descriptive statistics of groups in both the G004 and G005 study was performed. Among the 89 subjects in the test group of G004 study, the mean overall survival (OS) was 507.5 days; median OS was 364 days with standard deviation of 411.5 days. In the G004 control group of 45 subjects, the mean OS was 471.2 days; median OS was 333 days with a standard deviation of 373.0 days. Among the 12 subjects in the G005 test group, the mean OS was 507.8 days; median OS was 368.4 days with standard deviation of 461.1 days. In the G005 control group of 9 subjects, the mean OS was 397.1 days; median OS was 417.1 days with standard deviation of 216.0 days. Table 21 is a tabular form of the data presented above.

TABLE 21
Descriptive Statistics of Overall Survival in Patients Receiving
OT-101 or Standard Chemotherapy in G004 and G005 Studies.

| Study ID | # of | Mean OS | Median OS | SD |
|---|---|---|---|---|
| (Arm) | Subjects | (Days) | (Days) | (Days) |
| G004 (Test) | 89 | 507.5 | 364 | 411.5 |
| G004 (Control) | 45 | 471.2 | 333 | 373.0 |
| G005 (Test) | 12 | 507.8 | 368.4 | 461.1 |
| G005 (Control) | 9 | 397.1 | 417.1 | 216.0 |

For the non-inferiority study, a two-sample non-inferiority test for survival data using Cox Regression was performed. Specifically, the Wald Test, or 100(1-2α)% Confidence Interval Test for Non-Inferiority was used to see if the Upper 90.0% Confidence Limit (C.L) of the Hazard Ratio (HR) is within the non-inferiority hypothesis. The α-level was set at 0.050 with a hazard ratio (Hazard Ratio [HR]=Hazard [Treatment Group]/Hazard [Reference Group]) Non-Inferiority Bound of 1.25. Higher hazards were considered to be worse if they were greater than the bound for the non-inferiority hypothesis, H1:HR<Non-inferiority bound and the Efron Ties Method was used.

For an alternative hypothesis of HR<1.25, the hazard ratio was calculated to be 0.9168 with 90.0% confidence limit of hazard ratio at 0.6865˜1.2245 and a p-value of 0.0390. The Wald-Z value was determined to be −1.7621. In Cox Regression, the Hazard Ratio (HR) is commonly referred to as the Risk and is equal to Exp(B), where B is the estimated regression coefficient. For regression coefficients of the independent variable, B1 (Treatment=“Treatment”), the regression coefficient (B) was determined to be −0.086823 with a standard error of 0.175912. As listed above, the Risk Ratio, or Hazard Ratio was calculated to be Exp(B)=0.9168 with a mean of 0.6516.

Based on the results above, the non-inferiority of OT-101 was confirmed in terms of overall survival when compared to standard chemotherapy (TMZ, BCNU, or PCV) in both G004 and G005 as both the hazard ratio, which was 0.9168, and the 90% upper limit of the confidence interval, which was 1.2245, were both less than the initial non-inferiority bound of 1.25.

Among the control group, subjects receiving TMZ was most common with 43 subjects (36 subjects in G004 and 7 subjects in G005 study). An identical two-sample non-inferiority test for survival data using Cox Regression was performed for subjects that received either OT-101 or TMZ. With a non-inferiority alternative hypothesis determined as hazard ratio being less than 1.25, the hazard ratio was determined to be 0.7156 with a 90% confidence level between 0.5228˜0.9794 and a p-value of 0.0017. Wald's Z-value was determined to be −2.9231 and based on the obtained hazard ratio, it was concluded that OT-101 is non-inferior to TMZ.

The survival curves of OT-101, standard chemotherapy and both combined, were obtained utilizing NCSS software's Kaplan-Meier Curves (Logrank Tests).

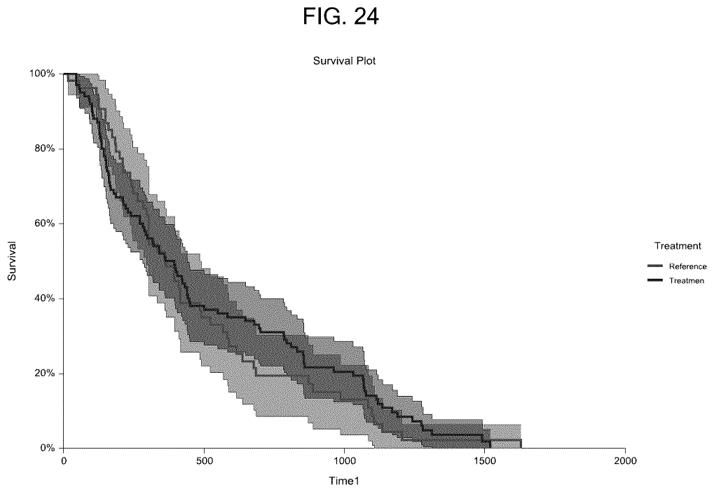

FIG. 24 shows the superimposed Kaplan-Meier survival curves of both OT-101 and standard chemotherapy. The survival curves of both drugs are well within each other's confidence interval range, which confirms non-inferiority of OT-101 when compared to standard chemotherapy in terms of overall survival.

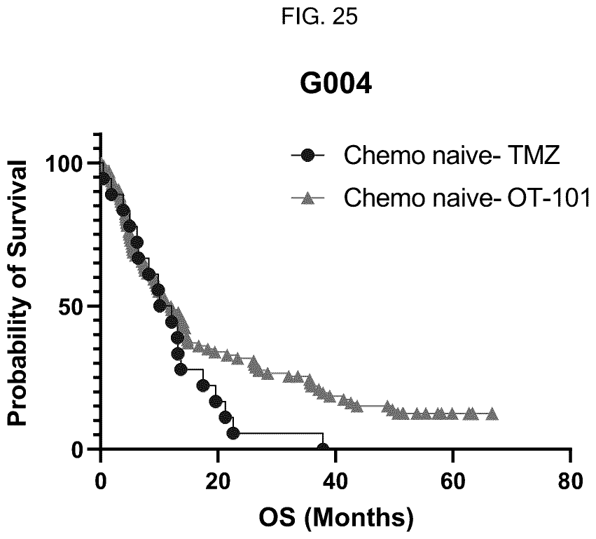

FIG. 25 shows chemotherapy-naive treated with temozolomide (TMZ), which confirms non-inferiority of OT-101 remained with TMZ added.

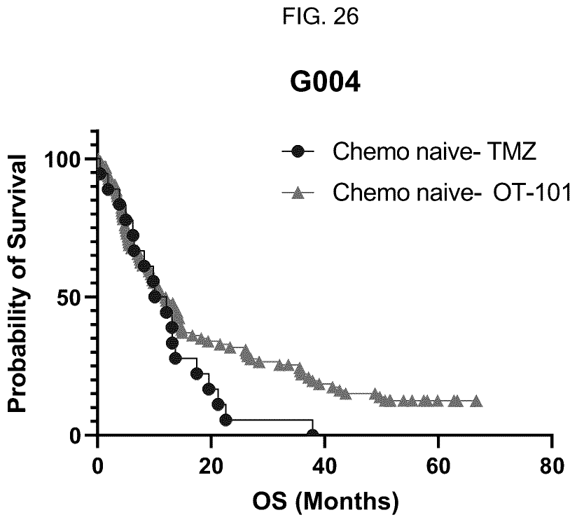

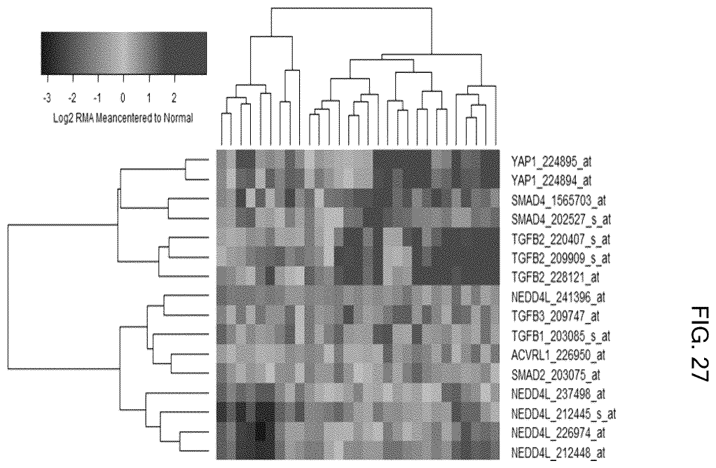

FIG. 26 shows chemotherapy-failure treated with chemotherapy agents CCNU/BCNU, which confirms non-inferiority of OT-101 with CCNU/BCNU added.

### Example 13

### TGF-β2 is a Valid Target for Therapy Against Gliomas.

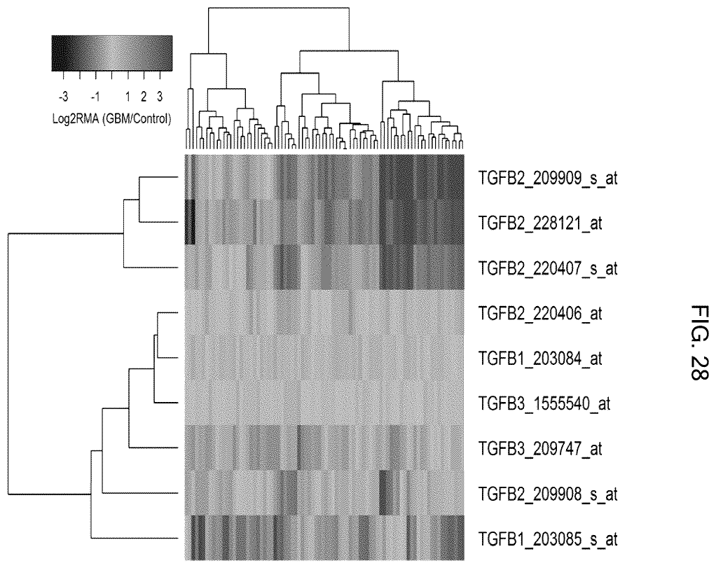

Three TGFB2 probesets exhibited increased levels of expression in DIPG patients (FIG. 27: Mean fold difference for Probeset 228121_at was 2.48 (Linear Contrast P-value=3.40×10-4); Probeset 220407_s_at was 2.00 (Linear Contrast P-value=0.006); Probeset 209909_s_at was 1.81 (Linear Contrast P-value=0.0185). One of the TGFB2 probesets was also exhibited the highest level of mean expression in DIPG patients; TGFB2_228121_at (mean=9.25±0.18 log 2 RMA); YAP1_224894_at (mean=8.55±0.19 log 2 RMA.

Results are summarized in Table 22.

TABLE 22
TGF-β2 is a valid target for therapy against gliomas

| Probeset | Gene | Fold Difference(vs. Normal) | Linear Contrast P-value |
|---|---|---|---|
| 228121_at | TGFB2 | 2.48 | 0.0003 |
| 1565703_at | SMAD4 | 2.14 | 0.0026 |
| 224895_at | YAP1 | 2.03 | 0.0052 |
| 220407_s_at | TGFB2 | 2.00 | 0.0060 |
| 224894_at | YAP1 | 1.99 | 0.0065 |
| 209909_s_at | TGFB2 | 1.81 | 0.0185 |
| 202527_s_at | SMAD4 | 1.73 | 0.0300 |

Expression of TGFB2 interactome represented probesets in pediatric DIPG patients compared to normal samples. The log 2 transformed fold difference values for each subject (columns (N=29)) and each probeset from the GEO archived dataset GSE26576 are depicted for DIPG patients mean centered to normal samples (N=2). The subjects and probesets were organized using a 2-way clustering algorithm using the average distance metric to determine co-regulation of all the probesets across patients and all patients across probesets. The heatmap depicts the most significantly up and down regulated probesets ranging from red to blue represents expression greater than normal to lower than normal in DIPG samples respectively. Fold difference and linear contrast p-values are depicted in the table showing 3 probesets for TGFB2 up-regulated in DIPG patients.

### Example 14

### TGF-β2 is a Valid Target for Therapy Against Pediatric GBM.

Similar results were obtained for pediatric GBM patients (FIG. 28). 3 probesets for TGFB2 significantly up-regulated greater than 2 fold in Pediatric GBM patients (Probesets: 209909_s_at (Fold change=4.03, P=1.78×10-5); 228121_at (Fold change=3.57, P=8.81×10-5); and 220407_s_at (Fold change=2.73, P=0.0019)). The three probesets with highest expression levels in pediatric GBM were: TGFB2_228121_at (mean=9.24±0.15); TGFB3_209747_at (mean=6.99±0.086); and TGFB1_203085_s_at (mean=6.80±0.16).

Results are summarized in Table 23.

TABLE 23
TGF-β2 is a valid target for therapy against pediatric GBM

| Probeset | Gene | Fold (Ped_GBMvsControl) | Linear_Contrast_P_value |
|---|---|---|---|
| 209909_s_at | TGFB2 | 4.029218 | 0.0000178 |
| 228121_at | TGFB2 | 3.567961 | 0.0000881 |
| 220407_s_at | TGFB2 | 2.728107 | 0.0019274 |
| 209747_at | TGFB3 | 1.358580 | 0.3419685 |
| 209908_s_at | TGFB2 | 1.277125 | 0.4480614 |
| 220406_at | TGFB2 | 1.253446 | 0.4835331 |
| 203084_at | TGFB1 | 1.222217 | 0.5336765 |
| 203085_s_at | TGFB1 | 1.095964 | 0.7762144 |
| 1555540_at | TGFB3 | 1.026191 | 0.9360745 |

Expression of TGFB1/2/3 probesets in Pediatric GBM patients compared to AO Patients (Control Group). The log 2 transformed fold difference values for each subject (columns (N=82)) and each of the 9 probesets from 4 GEO archived datasets (GSE19578 (N=25); GSE32374 (N=15); GSE34824 (N=27); and GSE49822 (N=15)) are depicted for Pediatric GBM patients mean centered to control samples (N=5; from GSE19578). The subjects and probesets were organized using a 2-way clustering algorithm using the average distance metric to determine co-regulation of all the probesets across patients and all patients across probesets. The heatmap depicts expression changes for each probeset ranging from red to blue representing expression greater than control to lower than control in Pediatric GBM samples respectively. Fold difference and linear contrast p-values are depicted in the table showing 3 probesets for TGFB2 significantly up-regulated greater than 2 fold in Pediatric GBM patients (Probesets: 209909_s_at (Fold change=4.03, P=1.78×10-5); 228121_at (Fold change=3.57, P=8.81×10-5); and 220407_s_at (Fold change=2.73, P=0.0019).

### Example 15

TGF-β2 is a Selective Biomarker which Predicts Improved Outcome for Cancer Radiation Therapy.

It has been discovered by the inventor herein that TGF-β2 can be used as a biomarker which selectively predicts improved outcome for radiation therapy in cancer.

A clinical study using CTGA Database showed the impact of TGF-β2 expression on overall survival and survival following radiation therapy. Surprisingly, significant survival advantage was observed with low TGF-β2 expressors versus high TGF-β2 expressors over all four quartiles of expression. No such differences were observed for TGF-beta-1 and TGF-beta-3.

Overall survival was studied on the pediatric brainstem subset and radiation therapy survival was performed on all gliomas patients treated with radiation. Again only TGF-β2 was predictive of survival. TGF-beta-1 and TGF-beta-3 were not predictive of survival.

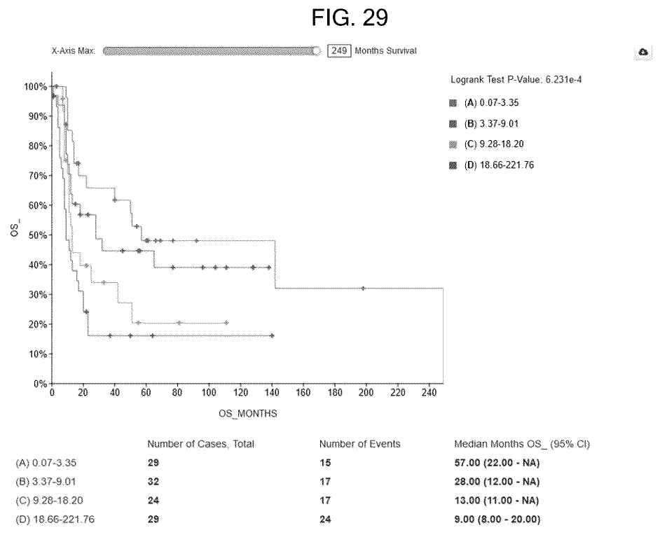

FIG. 29 shows that for gliomas in patients treated with radiation there was an Overall Survival advantage with low TGF-β2 expression. Only TGF-β2 was predictive of survival. Quartiles of TGF-β2 expression are indicated as A, B, C, and D.

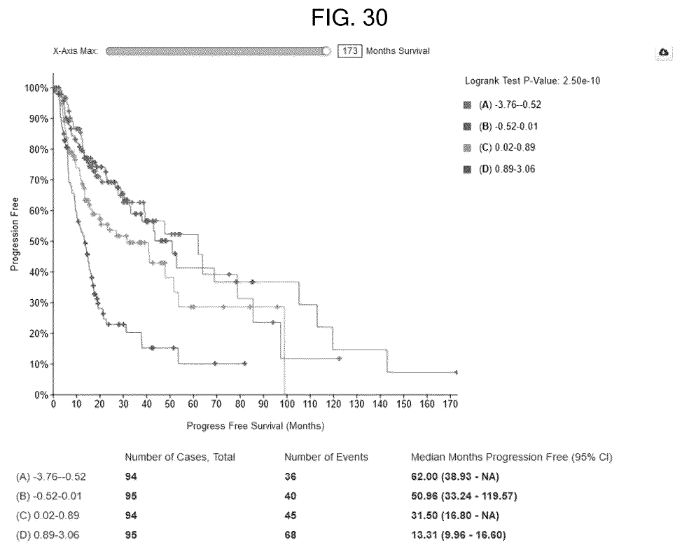

FIG. 30 shows that for gliomas in patients treated with radiation there was an Overall Survival advantage with low TGF-β2 expression. Only TGF-β2 was predictive of survival.

### Example 16

Reduced TGF-β2 Level is Selectively Predictive of Improved Overall Survival (OS) in Combination with Chemotherapy (TMZ), Chemotherapy (TMZ) and Radiation, or Antiangiogenic Therapy (Bevacizumab).

It has been discovered by the inventor herein that TGF-β2 can surprisingly be used as a biomarker which selectively predicts improved outcome in combination with chemotherapy (TMZ), TMZ and radiation, or antiangiogenic therapy (bevacizumab). No such predictive result was observed for TGF-β1 and TGF-β3.

A clinical study was performed using 23 datasets for gliomas to determine levels of TGF-β2 mRNA as predictive of survival following treatment of the agents of interest.

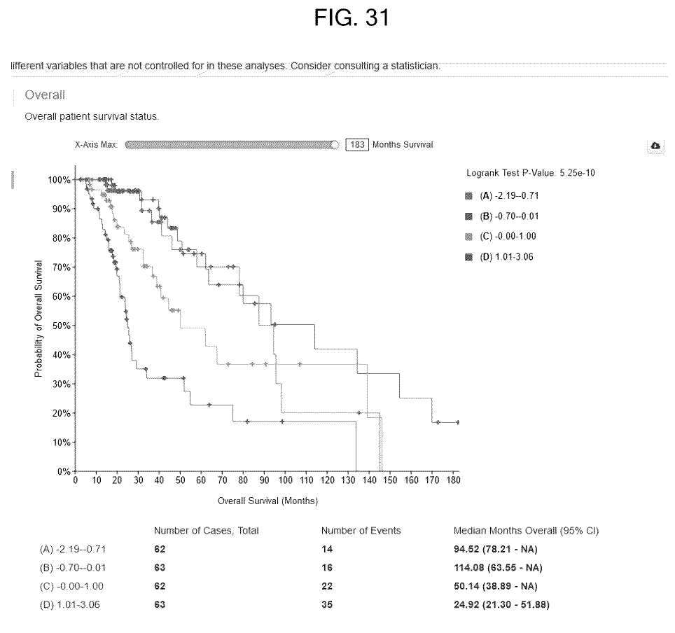

FIG. 31 shows TGF-β2 level is selectively predictive of improved overall survival (OS) in combination with chemotherapy (TMZ).

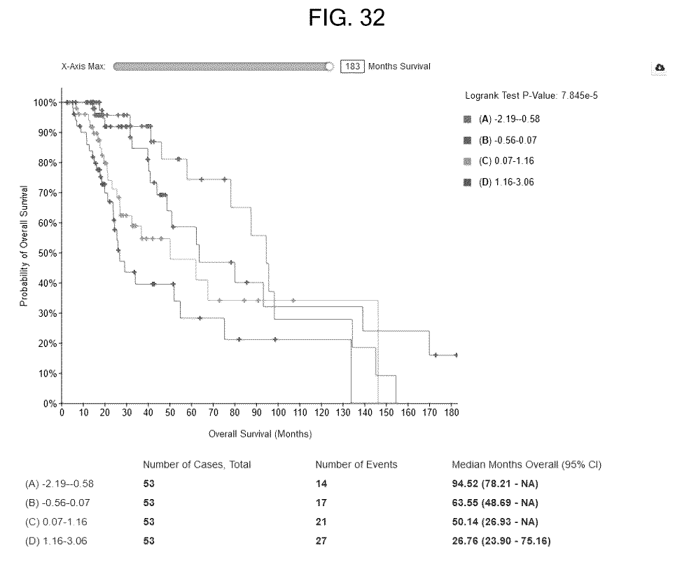

FIG. 32 shows TGF-β2 level is selectively predictive of improved overall survival (OS) in combination with chemotherapy TMZ and radiation.

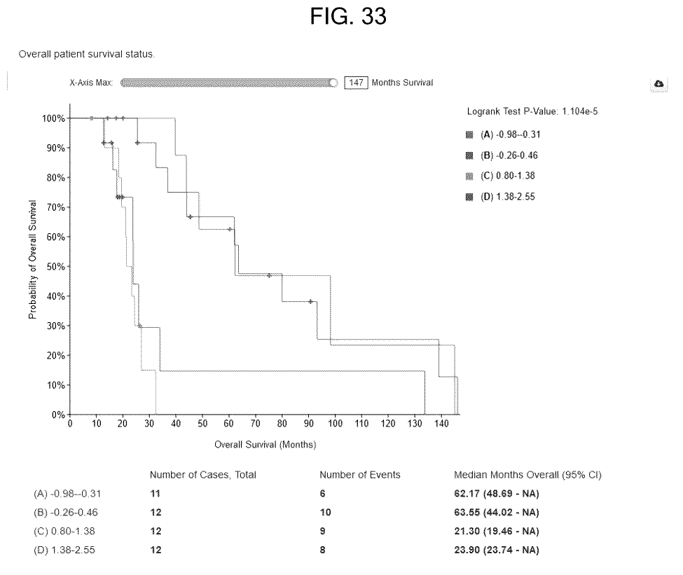

FIG. 33 shows TGF-β2 level is selectively predictive of improved overall survival (OS) in combination with antiangiogenic therapy (bevacizumab).

1-30. (canceled)

31. A device for delivering a fluid pharmaceutical composition by intracranial continuous infusion, the device comprising:
a reservoir [117] containing the pharmaceutical composition;
a pump [101] to force the pharmaceutical composition through an infusion tube [103] into an ommaya reservoir [111], the pump being in fluid communication with the ommaya reservoir, and wherein the pump is in fluid communication with the reservoir through a reservoir tube [115];
a filter [105] in line with the infusion tube; and
an entry catheter [113] in fluid communication with the ommaya reservoir, wherein the entry catheter is non-linear and has one or more bends to enter intraventricularly into a target region of the brain.

32. (canceled)

33. A device for delivering a fluid pharmaceutical composition by intracranial continuous infusion, the device comprising:
a reservoir [117] containing the pharmaceutical composition;
a pump [101] to force the pharmaceutical composition through an infusion tube [103] into an access port [107], the pump being in fluid communication with an ommaya reservoir [111], wherein the reservoir is in fluid communication with the pump through a reservoir tube [115];
a filter [105] in line with the infusion tube;
an indwelling tube [109] in fluid communication with the access port and the ommaya reservoir [111]; and
an entry catheter [113] in fluid communication with the ommaya reservoir, wherein the entry catheter is non-linear and has one or more bends to enter intraventricularly into a target region of the brain.

34. (canceled)

35. The device of claim 31, wherein the ommaya reservoir [111] comprises a partially-flexible top.

36. The device of claim 31, wherein the device provides continuous infusion of a therapeutically effective amount of the fluid pharmaceutical composition to the target region.

37. The device of claim 31, wherein the distal end of the entry catheter enters the target region of the brain.

38. The device of claim 31, wherein the ommaya reservoir [111] holds the pharmaceutical composition behind a membrane for a sustained period of time for sustained release of the pharmaceutical composition to the entry catheter.

39. The device of claim 31, wherein the distal end tip of the entry catheter that enters the brain is a step-down end, a recessed step end, a multi-port end, a microporous end, or a balloon tipped end.

40. The device of claim 31, wherein the pump [101] is a Pegasus Vario, a PEGA PCA, a CADD Solis VIP, a CADD-Legacy PLUS, a CADD-Legacy PCA, a CADD-Legacy 1, or other pump with similar specifications.

41. The device of claim 31, wherein the infusion rate of the fluid pharmaceutical composition is from 0.01 to 3000 ml/hr, or from 0.01 to 100 ml/hr, or from 0.01 to 2 ml/hr, or from 0.01 to 1 ml/hr, or from 0.05 to 0.5 ml/hr.

42. The device of claim 31, wherein the infusion tube [103] or the indwelling tube [109] is a PEGA Line 100 SF 100 cm with a 0.2 μm sterile filter, or a 200 cm infusion line with a 0.2 μm sterile filter, or a Port-a-Cath (#21-4034-24) with 22 G Needle (#21-2737-24) and extension (#21-7106-24), or other tube with similar specifications.

43. The device of claim 31, wherein the fluid pharmaceutical composition comprises an agent for inhibiting or suppressing expression of TGF-β, which can be used for treating or ameliorating the symptoms of a CNS disease in a human subject or animal.

44. The device of claim 31, wherein the fluid pharmaceutical composition comprises microparticles or nanoparticles of an agent, medicament, or delivery vehicle.

45. The device of claim 31, wherein the fluid pharmaceutical composition is therapeutic for CNS disease or CNS cancer.

46. The device of claim 31, wherein the fluid pharmaceutical composition is therapeutic for glioma, glioblastoma, diffuse intrinsic pontine glioma (DIPG), diffuse midline glioma (DMG), leptomeningeal or brain metastasis, brain or spinal cancer, or CNS tumors.

47. The device of claim 31, wherein the fluid pharmaceutical composition comprises an agent for inhibiting or suppressing expression of TGF-β2 which comprises CGGCATGTCTATTTTGTA SEQ ID NO:8 (OT-101) and chemically-modified variants thereof, LNA variants thereof, or gapmer variants thereof.

48. The device of claim 31, wherein the device is operated in combination with radiation therapy, or electric field therapy.

49. A device for delivering a pharmaceutical composition by intracranial continuous infusion, the device comprising:
a reservoir [402] containing the pharmaceutical composition, the reservoir comprising a hard shell [403], a flexible top [401], and a non-flexible mounting plate [405];
a port [407] in fluid communication with the reservoir; and
an entry catheter [413] in fluid communication with the reservoir, wherein the entry catheter is substantially linear and enters intraventricularly into a target region of the brain.

50. A device for delivering a pharmaceutical composition by intracranial continuous infusion, the device comprising:
a reservoir [502] containing the pharmaceutical composition, the reservoir comprising an upper hard shell [503], a lower hard shell [504], a flexible top [501], and a non-flexible mounting plate [505];
a port [507] in fluid communication with the reservoir for attaching an infusion line; and
a port [509] in fluid communication with the reservoir for attaching an entry catheter.

51. (canceled)

52. (canceled)

53. A method for administering a pharmaceutical composition by intracranial continuous infusion, comprising:
mounting a device according to claim 31 to a patient; and
pumping the pharmaceutical composition in the device to provide intracranial continuous infusion to the patient.

54. The method of claim 53, wherein the device is mounted and the entry catheter is placed into the brain without concurrent head or brain imaging.

55. The method of claim 54, wherein the device is mounted and a single entry catheter is placed into the brain.

56-60. (canceled)
